(19) United States
(12) Patent Application Publication
(10) Pub. No.: US 2024/0035016 A1
(43) Pub. Date: Feb. 1, 2024

(54) NUCLEIC ACID EXTRACTION AND ISOLATION WITH HEAT LABILE SILANES AND CHEMICALLY MODIFIED SOLID SUPPORTS

(71) Applicant: Cepheid, Sunnyvale, CA (US)

(72) Inventors: Alex I. KUTYAVIN, Sunnyvale, CA (US); Kevin P. LUND, Sunnyvale, CA (US); Michael REED, Sunnyvale, CA (US); Cameron J. NAKATANI, Sunnyvale, CA (US); Oliver Z. NANASSY, Sunnyvale, CA (US); Richard J. LEUZZI, Sunnyvale, CA (US)

(21) Appl. No.: 18/309,691

(22) Filed: Apr. 28, 2023

Related U.S. Application Data
(60) Provisional application No. 63/337,014, filed on Apr. 29, 2022.

(51) Int. Cl. C12N 15/10; B01L 3/00

(52) U.S. Cl. CPC C12N 15/1006; B01L 3/502761; B01L 2200/0647; B01L 2200/16; B01L 2300/0681; B01L 2300/12

(57) ABSTRACT

Compositions and methods for isolating and detecting nucleic acid in a biological sample are provided. The compositions and methods utilize a modified solid support comprising an amine or amide group.

### CROSS REFERENCE TO RELATED APPLICATIONS

The present application claims priority to, and the benefit of, U.S. Provisional Application No. 63/337,014, filed on Apr. 29, 2022, which is incorporated by reference herein for all purposes.

### FIELD OF THE DISCLOSURE

The invention relates generally to the field of molecular biology. In certain embodiments the invention provides devices, kits, and methods relating to the isolation and detection of nucleic acids.

### BACKGROUND

Isolating nucleic acids is typically the first step of most molecular biological inquiries including polymerase chain reaction (PCR), DNA hybridization, restriction enzyme digestion, DNA sequencing, and array-based experiments. As such, there is a need for simple and reliable methods for isolating nucleic acid, and in particular, for isolating high quality nucleic acid. A variety of techniques for isolating nucleic acids from a sample have been described, one of the most common involving lysing the nucleic acid source in a chaotropic substance (for example, guanidinium salt, urea, and sodium iodide), in the presence of a DNA binding solid phase (for example, glass beads or fibers). The released nucleic acid is bound to the solid phase in a one-step reaction, where the solid phase is washed to remove any residual contaminants. As an example, glass fiber disc having a 30 mm diameter, 0.7 μm pore size has been shown to capture 150 μg of plasmid DNA (binding capacity about 30 μg/cm²) with 2 M guanidine hydrochloride (GuHCl) lysates. Kim, Y—C and Morrison, S. L. PLoS ONE (2009) 4, 11, e7750. Although these methods have proven to be fast, they have resulted in a moderate level of DNA shearing and some level of contamination. Residual GuHCl or GuSCN can poison downstream PCR reactions and must be removed with extensive washing. There is a need for methods of isolating a nucleic acid from a sample that are fast, economical, and produce high yields.

In order to increase sensitivity of nucleic acid detection, large sample volumes can be prepared. The preparation of large volumes, however, is contradictory to fluidic systems for automatic lysis, processing and/or analysis of biological samples. Particularly, current automative on-market products for processing samples, such as whole blood for the detection of pathogenic targets are limited to tolerating only a few hundred microliters of blood per test. Existing technologies capable of processing multiple milliliters of blood samples for pathogen detection involves a laborious process with multiple manual steps that must be followed correctly by the user, including centrifugation, decantation, vortexing, and glass column-based DNA precipitation and purification. After using the existing sample processing kit, the user is then still responsible for preparing a PCR set-up that can accurately analyze what is produced by the sample kit. There is a market need for automated sample processing of multiple milliliters of blood within a single device. It would also be useful if the method minimized the required manipulation of the sample and could be performed using a single device. Certain embodiments of the invention described herein provide for such methods. Other embodiments of the invention described herein provide for devices and kits which may be used for isolating nucleic acids from a sample. Still other embodiments of the invention provide for the detection of a nucleic acid in a sample.

### SUMMARY

Described herein are compositions, systems, and methods for isolating and purifying nucleic acid from a biological sample. The compositions, systems, and methods utilize a modified solid support comprising a DNA binding ligand as a separating material, thereby reducing and/or eliminating the amount of lysis reagents and nucleic acid binding agents conventionally used in PCR processes.

In some aspects, the compositions and systems for isolating a nucleic acid from a biological sample comprise: a compound bonded to a solid support, the compound being derived from a structure represented by the formula:

Y-(L)_{y}-SiX₃
wherein, Y is a DNA binding ligand selected from an alkylamine, a cycloalkylamine, an alkyloxy amine, a polyamine moiety, an intercalating agent, a minor groove binder (e.g., a bisbenzimide minor groove binder), a peptide, an amino acid, an arylamine, or a combination thereof; L is a linker selected from an alkyl group, a heteroalkyl group, an alkene group, a heteroalkene group, a polyacrylic acid, a Diels-Alder adduct, or a combination thereof, each X, independently for each occurrence, is selected from a hydrolyzable group, an alkyl group, a heteroalkyl group, an alkenyl group, or two or three Xs combine to form one or more cyclic groups, or one X combines with Y to form a cyclic azasilane; and y is 0 or 1.

The DNA binding ligand or the substituent Y can comprise a plurality of amine groups; a plurality of amide groups; or a combination thereof. For example, the DNA binding ligand or Y can comprise at least two, at least three, at least four, at least five, at least six amine or amide groups, or a combination thereof. In some embodiments, the DNA binding ligand or Y comprises an alkylamine group, an imidazole group, or a combination thereof. Representative examples of the amine group include spermine, methylamine, ethylamine, propylamine, ethylenediamine, diethylene triamine, 1,3-dimethyldipropylenediamine, 3-(2-aminoethyl)aminopropyl, (2-aminoethyl)trimethylammonium hydrochloride, tris(2-aminoethyl)amine, or a combination thereof.

The linker, L, is present (or y is 1) in some aspects of the compound disclosed herein. The linker can be selected from an alkyleneoxy group, an alkylene group, or a Diels-Alder adduct.

The group X facilitates attachment of the compounds disclosed herein with the solid support. FIG. 4, for example, shows how individual silane molecules can be incorporated into a solid support material by bonding to the solid support material and/or with neighboring silane molecules. In some embodiments, each X, independently for each occurrence, can be selected from a moiety that facilitates hydrogen bonding, electrostatic attraction, covalent bonding, horizontal and/or vertical polymerization, with functionalities present at the interface or the solid support material and/or with neighboring silanes. In some aspects of the compositions and systems, at least two Xs can be independently selected from a halogen, an alkoxy, a dialkylamino, a trifluoromethanesulfonate, or combine together with the Si atom to which they are attached to form an oligomeric or polymeric silane, a silatrane, a cyclic siloxane, a polysilsesquioxane, or a silazane. For example, at least two Xs can be independently selected from an alkoxy group (such as ethoxy or methoxy).

In other aspects, the compositions and systems for isolation of a nucleic acid from a biological sample comprises a Diels-Alder adduct, the Diels-Alder adduct including a DNA binding ligand, wherein the Diels-Alder adduct is optionally bonded to the solid support. As defined herein, the DNA binding ligand can comprise an amine group, an intercalating agent, a minor groove binder, a peptide, an amino acid, or a combination thereof. In some examples, the DNA binding ligand is selected from an alkylamine, a cycloalkylamine, an alkyloxy amine, an arylamine, a polyamine moiety, or a combination thereof. The Diels-Alder adduct described herein can be derived from an unsaturated cyclic imido group.

In some embodiments, the compound or the Diels-Alder adduct bonded to the solid support can be derived from a structure represented by the general Formula,

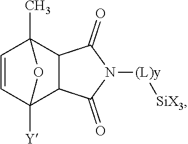

their isomers, salts, tautomers, or combinations thereof, wherein Y′ is the DNA binding ligand, and L, X, and y are as defined herein. For example, Y′ can comprise an alkylamine group, an imidazole group, or a combination thereof. In some examples, Y′ can further comprise more than one DNA binding ligand, such as in FIG. 8, which shows an alkylamine group further linked to a minor groove DNA binding ligands. L is optionally present and can be selected from an alkyleneoxy group, an alkylene group, cyanuric chloride, an alkylamine, or a combination thereof. At least two Xs can be independently selected from a halogen, an alkoxy, a dialkylamino, a trifluoromethanesulfonate, or combine together with the Si atom to which they are attached to form a silatrane, a cyclic siloxane, a polysilsesquioxane, or a silazane.

In some examples, the compound or the Diels-Alder adduct can be derived from one of the following structures:

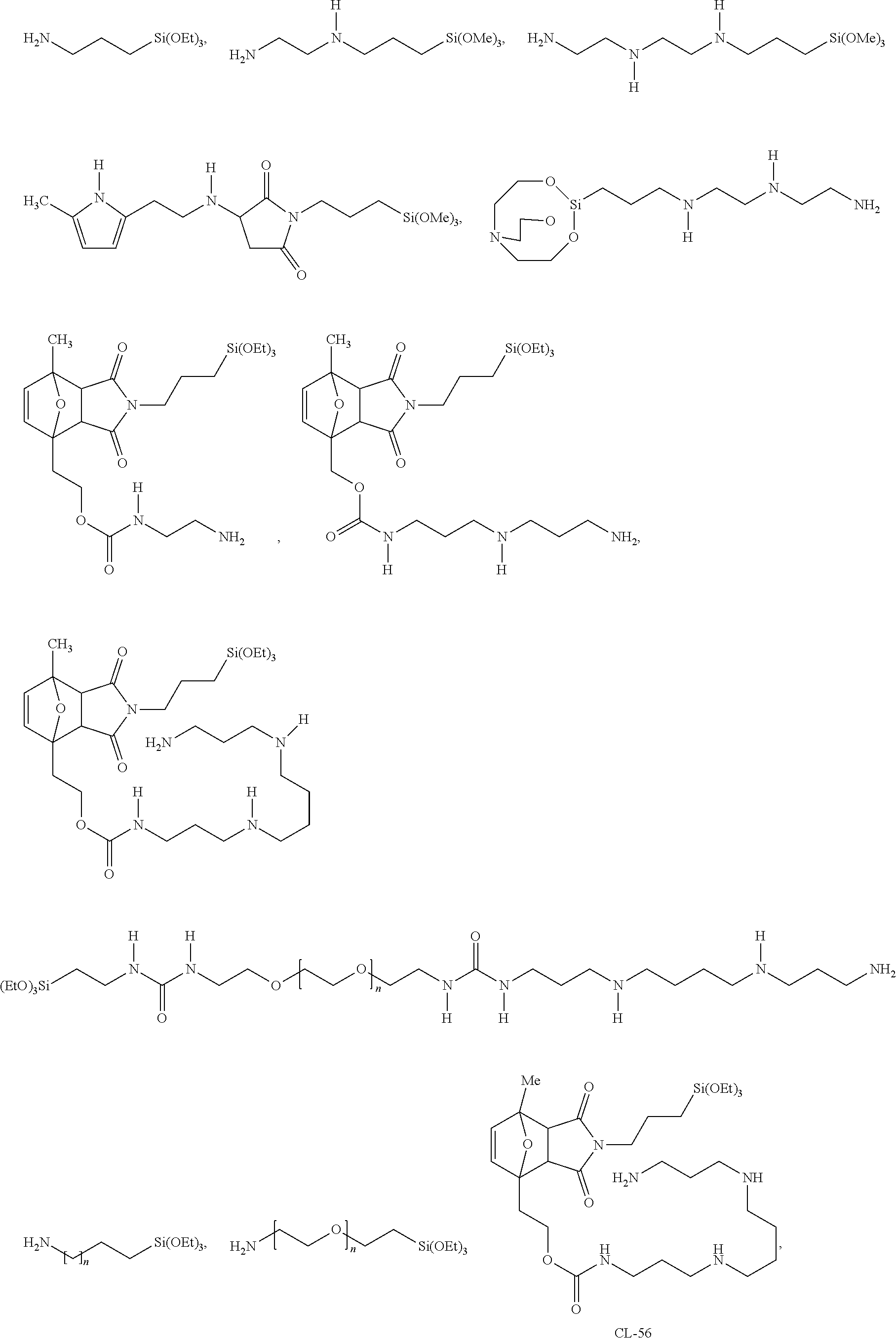

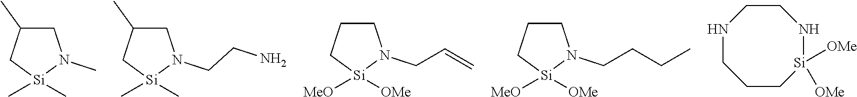

3-aminopropyltrimethoxysilane, 3-aminopropyltriethoxysilane, an aminoalkylsilatrane, 3-(2-aminoethyl)aminopropyltriethoxysilane, 3-(2-aminoethyl)aminopropyltrimethoxysilane, or a combination thereof, and wherein n is an integer from 0 to 10, from 1 to 10, or from 1 to 5.

The solid support described herein can comprise a material selected from silica, glass, ethylenic backbone polymer, mica, polycarbonate, zeolite, titanium dioxide, magnetic bead, glass bead, cellulose filter, polycarbonate filter, polytetrafluoroethylene filter, polyvinylpyrrolidone filter, polyethersulfone filter, glass fiber filter or a combination thereof.

In some examples, the solid support is a glass fiber filter. The glass fiber filter can have a pore size selected to accommodate correspondingly sized beads to facilitate mechanical lysis. For example, the glass fiber filter can have a pore size from 0.2 μm to 3 μm, from 0.2 μm to 2 μm, from 0.5 μm to 1.0 μm, or from 0.6 μm to 0.8 μm. Further, the glass fiber filter can have a basis weight from 35 g/m² to 100 g/m², preferably from 50 g/m² to 85 g/m², or from 70 g/m² to 80 g/m². The glass fiber filter can have a fiber diameter from 1 μm to 100 μm, preferably from 1 μm to 50 μm, or from 1 μm to 25 μm. The glass fiber filter can have a thickness from 250 μm to 2,000 μm, from 300 μm to 1,500 μm, from 300 μm to 1,000 μm, from 300 μm to 750 μm, or from 350 μm to 500 μm. As described herein, the glass fiber filter can accommodate beads to facilitate mechanical lysis The beads can include glass beads, silica beads, or a combination thereof. The compound or the Diels-Alder adduct can be bonded to the solid support via a siloxane bridge, a carboxylate bridge, as ester bridge, an ether bridge, or a combination thereof.

Separating materials for nucleic acid isolation and purification are also disclosed herein. The separating material can comprise a compound or compositions disclosed herein, comprising a DNA binding ligand. For example, the separating material can comprise a glass fiber solid support and a compound bonded to the glass fiber solid support. As described herein, the compound can be derived from a structure represented by the formula: Y-(L)_{y}-SiX₃, and wherein Y, L, X, and y are as defined herein. In other examples, the separating material can comprise a glass fiber solid support comprising a Diels-Alder adduct having a DNA binding ligand, wherein the adduct is chemically bonded to a glass fiber solid support. The glass fiber solid support may further comprise a polymeric binder. In some examples, the glass fiber solid support can be in the form of a 500 microns to 2000 microns thick glass fiber disk having an effective pore size of 0.5 microns to 1 micron.

Systems comprising the compounds, compositions, and separating materials disclosed herein are also provided. The systems can be a sample cartridge, preferably an automated sample cartridge. Accordingly, disclosed herein are systems comprising a sample cartridge for isolation and detection of nucleic acid from a biological sample. The sample cartridge can comprise: a) a cartridge body having a plurality of chambers defined therein, wherein the plurality of chambers are in in fluidic communication through a fluidic path of the cartridge, and wherein at least one chamber is configured to receive the biological sample, b) a reaction vessel configured for amplification of the nucleic acid by thermal cycling, and c) a filter disposed in the fluidic path between the plurality of chambers and the reaction vessel, wherein the filter comprises a compound, separating material, or composition as disclosed herein, wherein the plurality of chambers and the reaction vessel independently comprise reagents for releasing nucleic acid from the biological sample, and primers and probes for detection of the nucleic acid. In other aspects, the sample cartridge can comprise a) a cartridge body having a plurality of chambers therein, wherein the plurality of chambers include: a sample chamber having at least a fluid outlet in fluid communication with another chamber of the plurality; and a lysis chamber in fluidic communication with the sample chamber, the lysis chamber comprising reagents for releasing nucleic acid, optionally wherein the sample chamber and lysis chamber are the same; b) a reaction vessel fluidically coupled to the plurality of chambers of the cartridge body and configured for amplification of nucleic acid and ii) detection of a plurality of amplification products; c) a filter disposed in the fluidic path between the lysis chamber and the reaction vessel, wherein the filter comprises a solid support modified with a DNA binding ligand selected from an alkylamine, a cycloalkylamine, an alkyloxy amine, a polyamine moiety, an intercalating agent, a minor groove binder (e.g., a bisbenzimide minor groove binder), a peptide, an amino acid, a protein, an arylamine, or a combination thereof, and d) a plurality of primers and/or probes disposed in one or more chambers of the plurality of chambers or reaction vessel for detection of the nucleic acid. The sample cartridge is configured to carry out non-isothermal amplification such as by thermal cycling, gradient (temperature differential), or temperature oscillation, and isothermal amplification.

The sample cartridge may further comprise a syringe that is movable to facilitate fluid flow into and from the lysis chamber by fluctuation of pressure.

In some aspects of the sample cartridge, the lysis chamber may further comprise a valve body and a valve cap, wherein the valve body interfaces with the valve cap to define the lysis chamber therebetween, and wherein the filter is held within the lysis chamber secured between the valve body and the valve cap. The lysis chamber can have a fluid flow path between an inlet in the cap and an outlet in the valve body that is fluidically coupled to a fluid displacement region of the valve body, wherein the fluid displacement region is depressurizable by movement of the syringe to draw fluid into the fluid displacement region and pressurizable by movement of the syringe to expel fluid from the fluid displacement region.

The lysis chamber optionally comprises lysis reagents, the lysis reagents selected from a chaotropic agent, a chelating agent, a buffer, a detergent, or combinations thereof, to facilitate chemical lysis. The cartridge body can further comprise an ultrasonic, piezoelectric, magnetostrictive, or electrostatic transducer, for example in the lysis chamber to facilitate mechanical lysing. The cartridge body in one or more of the plurality of chambers may further comprise a binding agent.

The disclosed compositions and systems provide improvements in performance of the sample cartridge disclosed herein by facilitating reduction and/or eliminating the amount of lysis reagent and binding reagent conventionally used in cartridge design. Particularly, the sample cartridges disclosed herein have reduction in the amount of chaotropic agent and/or PEG contained in lysis buffer, wash buffer, elution buffer, and binding reagent. Furthermore, the volume of lysis buffer, wash buffer, elution buffer, and binding reagent stored within the cartridge can be reduced.

In some prior experiments, the maximum fluid volume that could be processed with conventional cartridges was 300 μL at which volume pressure aborts (at 100 psi or greater) occurred approximately 50% of the time, thus volumes in conventional cartridges were reduced and limited to 125 μL to avoid pressure aborts. The current cartridges allow for higher sample volumes (e.g., 300 μL, 700 μL, 1,000 μL, up to 5,000 μL) without reaching or exceeding the maximum pressure allowable (100 psi). The attribute of the cartridge that allowed for the processing of higher volumes was related to the reduction in chaotropic agent and binding agent as well as the filter material. Flow rates for these experiments were 10 μL per second for conventional cartridges. Flow rates are limited by pressure in sample cartridges, but the compounds and compositions in the disclosed sample cartridges allow for flow rates up to at least 100 μL per second. Accordingly, more total sample are able to be processed with the disclosed cartridges as compared to conventional cartridges in less time while maintaining viable pressures below 100 psi. overall, the disclose sample cartridge together with the reagents can allow for higher flow rates up to about 100 μL per second, such as from about 10 μL to about 100 μL, compared to conventional cartridges. The disclosed sample cartridge together with the reagents can allow for pressure below 100 psi, below 80 psi, or below 60 psi. The disclosed sample cartridge can allow for sample volumes up to 5000 μL, such as from 300 μL to 5,000 μL, from 300 μL to 3,000 μL, from 300 μL to 2,000 μL, or from 300 μL to 1,000 μL.

The cartridge can be a single-use disposable cartridge. In some embodiments, the cartridge is an automated cartridge.

Methods for isolating a nucleic acid from a biological sample are also provided. The method can comprise (a) causing the nucleic acid to contact a compound bonded to a solid support as provided in the compositions and systems disclosed herein, and (b) eluting the nucleic acid from the solid support. In other aspects, the methods for isolation of a nucleic acid from a biological sample comprises (a) causing the nucleic acid to contact a composition comprising a Diels-Alder adduct, the Diels-Alder adduct including a DNA binding ligand as disclosed herein, and (b) concentrating the nucleic acid onto a solid support. In some aspects, the method for detecting nucleic acid in a biological sample obtained from a subject can comprise a) contacting nucleic acid from the biological sample with a set of primers and optional probes in a sample cartridge as described herein; b) subjecting the nucleic acid, primer pairs, and optional probes to amplification conditions; c) detecting the presence of amplification product(s), optionally via real-time PCR, melt curve analysis, or a combination thereof, and d) detecting the presence of the nucleic acid in the biological sample based on detection of the amplification products. Said contacting nucleic acid from the sample with the set of primers and optional probes in a sample cartridge can comprise placing the biological sample in the cartridge comprising a cartridge body having a plurality of chambers in fluidic communication, a reaction vessel having one or more reaction chambers and configured for amplification of the nucleic acid, a fluidic path between the plurality of chambers and the reaction vessel, and a filter in the fluidic path, and if the biological sample comprises cells, lysing cells in the biological sample with one or more lysis reagents present within at least one of the plurality of chambers. Said subjecting the nucleic acid, primer pairs, and optional probes to amplification conditions can comprise amplifying the nucleic acid with primers and probes present in solution within at least one of the plurality of chambers. Said subjecting the nucleic acid, primer pairs, and optional probes to amplification conditions can comprise amplifying the nucleic acid with primers and probes present in solution within at least one of the plurality of chambers.

The methods are used for isolating and purifying nucleic acid from a biological sample. The biological sample can be selected from blood, plasma, serum, semen, spinal fluid, tissue, tear, urine, stool, saliva, smear preparation, respiratory sample, nasopharyngeal sample, vaginal swab, vaginal mucus sample, vaginal tissue sample, vaginal cell sample, bacterial culture, mammalian cell culture, viral culture, human cell, bacteria, extracellular fluid, pancreatic fluid, cell lysate, PCR reaction mixture, or in vitro nucleic acid modification reaction mixture. In some embodiments, the biological sample is blood, plasma, respiratory sample, or vaginal swab. In some examples, the biological sample comprises nucleic acid selected from genomic DNA, total RNA, short-DNA, small DNA, tumor-derived nucleic acid, methylated DNA, microbial nucleic acid, bacterial nucleic acid, viral nucleic acid, cell free nucleic acid, or combinations thereof. In some embodiments, the biological sample comprises cell free nucleic acid.

In the methods disclosed herein, the biological sample may be contacted with a buffer prior to or simultaneously with step a) causing the nucleic acid to contact the composition or compound bonded to a solid support. The buffer can be a lysis buffer and include one or more of a chaotropic agent, a salt, a buffering agent, a surfactant, a defoaming agent, a binding agent, a precipitating agent, or a combination thereof. The chaotropic agent can be selected from guanidinium thiocyanate, guanidinium hydrochloride, alkali perchlorate, alkali iodide, urea, formamide, or combinations thereof. The chaotropic agent can be utilized at a lower concentration compared to conventional lysis assay. For example, the chaotropic agent can be used at a concentration of less than 4.5 M, less than 2 M, or less than 1 M. of the lysis buffer. In some embodiments, the methods disclosed herein do not utilize a chaotropic agent or a lysis buffer.

When a chaotropic agent or lysis buffer is not used in the methods disclosed herein, the biological sample can be contacted with a buffer comprising saline (inorganic salts such as CaCl₂), MgSO₄, KCl, NaHCO₃, NaCl, etc.), phosphate buffer, Tris buffer, 2-amino-2-hydroxymethyl-1,3-propanediol, HEPES, PBS, citrate buffer, TES, MOPS, PIPES, Cacodylate, SSC, MES, saccharide or disaccharide, or combinations thereof. For example, the buffer can be a commercially available buffer such as Hanks' Balanced Salt Solution available from Sigma Aldrich or TE Buffer available from Fisher BioReagents.

In some embodiments, the methods further comprise contacting the nucleic acid with a binding agent, a filtering reagent, a washing reagent, or a combination thereof, simultaneously with concentrating or prior to eluting the nucleic acid. The binding reagent (such as PEG or a salt) can promote binding of nucleic acids to the filter while removing non-target material. The filtering agent and/or the washing agent may comprise the binding agent. The binding agent can be utilized at a lower concentration compared to conventional lysis assay. For example, the binding agent can be used at a concentration of less than 40% v/v, less than 30% v/v, less than 20% v/v, or less than 10% v/v, of the filtering agent and/or the washing agent. In some embodiments, the compositions, systems, and methods disclosed herein do not utilize a binding agent. In some instances, neither lysis buffer (or a chaotropic agent) nor binding reagents are used in the methods disclosed herein. In such cases, proteinase K or a mechanical treatment such as sonication can be used to release nucleic acids from the sample which subsequently bind to the modified filter.

Eluting may comprise heating the nucleic acid on the filter to a temperature of 100° C. or less, 95° C. or less, 85° C. or less, 75° C. or less, 65° C. or less, 55° C. or less; sonicating the nucleic acid; photochemically cleaving the compound/composition; or a combination thereof, in the presence of an eluting agent. In some embodiments, the methods for isolating and purifying a nucleic acid can comprise eluting the nucleic acid with an eluting agent. The eluting agent can have a pH greater than about 9, greater than about 10, greater than about 11, or greater than about 12. The eluting agent can have a pH greater than about 10. The eluting agent can have a pH of about 10 to about 13. In some Examples provided herein, 50 mM KOH (pH 12.7) was used for eluting the nucleic acid. The use of high pH to elute nucleic acid such as DNA is unique especially to the cartridges described herein and provides improved speed and performance of the disclosed methods. Speed is provided by the rapid neutralization of acidic ammonium ions by the high concentration of hydroxide ions. Alkylamines have a pKa ˜10-11 and are immediately deprotonated at pH 12.7, to form the neutral free base on the solid surface, and release the cationic DNA. A further advantage of the high pH is the denaturing effect of KOH on captured DNA or RNA. Acidic functional groups in the heterocyclic bases of DNA or RNA are immediately deprotonated and cannot form Watson-Crick bonds. Double stranded structures and other secondary structures are disrupted, but can re-nature when neutralized for example, with Tris HCl. This chemical denaturing of captured genomic DNA can be an advantage for isothermal assays that do not undergo the usual heat denaturing of PCR. The cartridges provided herein allows for rapid neutralization of eluted DNA or RNA in KOH followed by reaction with Tris to produce a final pH of about 8.5 for downstream PCR or other nucleic acid assays. In some embodiments, the eluting agent can have a pH less than about 9, less than about 8.5, or less than about 8. This lower pH elution of bound DNA or RNA can be an advantage, especially for devices that don't facilitate rapid neutralization of the KOH solution. It is known that RNA is hydrolyzed by high pH, so short exposure times to KOH are important for good quality RNA. In some examples, the eluting agent comprises a polyanion, a polycation, ammonia or an alkali metal hydroxide. For example, the eluting agent may comprise a polyanion such as a carrageenan, a carrier nucleic acid, or a combination thereof.

Methods for detecting a nucleic acid in a biological sample are also disclosed. The methods can include (a) isolating the nucleic acid from the biological sample using a method as defined herein; (b) eluting the nucleic acid from the solid support with an eluting agent; and (c) detecting the nucleic acid. Detecting the nucleic acid can comprise amplifying the nucleic acid by polymerase chain reaction. The polymerase chain reaction can be selected from a nested PCR, an isothermal PCR, qPCR, or RT-PCR.

In other embodiments, the methods for detecting a nucleic acid in a biological sample can include placing the biological sample in a cartridge body as disclosed herein, lysing cells optionally with one or more lysis reagents present within at least one of the plurality of chambers and capturing nucleic acid released therefrom; and amplifying the nucleic acid with primers and probes for detecting the presence of the nucleic acid. The nucleic acid can be detected within the biological sample within 75 minutes or within 60 minutes of collecting the sample from the subject.

### BRIEF DESCRIPTION OF THE FIGURES

The application file contains at least one drawing executed in color. Copies of this patent or patent application publication with color drawing(s) will be provided by the Office upon request and payment of the necessary fee.

FIG. 1A shows attachment of DNA binders to glass. The image on the left shows reactive groups such as amines, carboxylic acids and epoxides are attached to glass surfaces using silane reagents. Aliphatic amines can bind DNA directly. The silane density, thickness, crosslinking, reactive groups, surface charge or surface density can be varied to promote faster extraction, exclude/reduce amounts of a binding agent (such as PEG) or chaotropic agent (such as guanidine thiocyanate). The types of rea may also be varied. The image on the right shows other DNA binding ligands conjugated to glass via a linker. The DNA binder type, linker type, loading density, surface charge or surface density can be varied to promote faster extraction, exclude/reduce amounts of a binding agent (such as PEG) or chaotropic agent (such as guanidine thiocyanate).

FIG. 1B shows a scanning electron micrograph (SEM) of glass fiber filter (GFF). GFF of specified pore size (0.7 um) and thickness (0.43 mm) is standard for EPA compliant leaching assays. The filter materials are supplied as round discs of various diameter and are used as received from the manufacturer for the silanization methods described here.

FIG. 2 shows a scheme for synthesis of PFP ester reagent, and DMT assay for measuring surface alkylamine groups. Accessible surface amino groups on the glass surface react with DMT (dimethoxytrityl) containing PFP ester. DMT cation releases with acid treatment and absorbance is measured.

FIG. 3 shows measurement of surface amine density in aminopropyl (AP) coated glass fiber filters (AP-GFF). AP-GFF were prepared using Method B as detailed in the examples. For kinetics experiment, 0.33 mm thick, 1 μm pore size AP-GFF filters were treated with 0.1 M PFP ester.

FIG. 4 shows an APTES-derived solid support layer: individual silane molecules can be incorporated into the layer via (a) hydrogen bonding, (b) electrostatic attraction, (c) covalent bonding with the substrate, (d) horizontal and (e) vertical polymerization with neighboring silanes, and (f) oligomeric/polymeric silanes can also react/interact with functionalities present at the interface. The figure shows multilayer coating, crosslinking, and non-covalent bonding occurs at the surface.

FIG. 5 shows rigid linker in CL silanes increases (such as double) amine density vs. aliphatic amines. The structure of flexible aliphatic DETA and rigid CL54 linker structures are also shown in FIG. 5. Both linkers can form 3 ionizable alkylammonium groups to ionically bind the polyanionic DNA. Structure of an ionically “captured” DNA dinucleotide illustrates relative dimensions of the silanized glass surface.

FIG. 6 shows a comparison of EDA and APTES coated GFF (Method A). These data show DNA extraction efficiency with the 2 amine coating types. The 2% and 4% APTES showed slightly lower capture efficiency. In this example, EDA retained DNA better at all concentrations. Amine loading values (nmole/cm²) are as follows: APTES: 2% is at 32.5, 4% is at 33.3, 6% is at 31, 8% is at 35. EDA: 0.5% is at 48, 1% is at 50, 2% is at 45, and 4% is at 47.

FIG. 7 shows EDC coupling of BisTris to succ-AP-GFF. Aminopropyl GFF is first succcinylated to provide carboxylic acid coated surface. EDC activation and coupling to Bis-Tris gives ester coating. At pH 6, unprotonated amine of Bis-Tris can also react.

FIG. 8 shows AP-GFF coated with a minor groove binding DNA ligand. Bis-benzimide with attached hexylamine linker (BB—NH₂) was prepared and conjugated to AP-GFF using a novel cyanuric chloride (CC) activated surface (CC-GFF). BB-CC-AP-GFF binds DNA efficiently, but it does not elute at high pH.

FIG. 9 shows heat release (top) and alkaline release (bottom) of DNA. Glass Fiber Filters are first silanized with cleavable linker silane (CL53) to give CL53-GFF (top). The amine coating is released from GFF by heating at 95° C. for a few minutes. CL53-GFF was further coated with bis-benzimide ligands. DNA capture/heat release was demonstrated. Alternately, GFF are first silanized with APTES (bottom). This coating binds DNA at pH<10 and releases at pH >12.

FIGS. 10A-10C show an overview of a sample cartridge with a valve assembly configured for performing differing sample processes, including chemical lysing of targets, which is configured for immuno-PCR and optional integrated nucleic acid analysis of the target assay panel in accordance with some embodiments of the invention. FIG. 10A shows the sample cartridge body with reaction vessel, FIG. 10B shows an exploded view of the sample cartridge, and FIG. 10C shows components of the valve assembly, in accordance with some embodiments.

FIG. 11A illustrates various valve assemblies A, B, C, and a universal cartridge each suited for one or more types of target lysing, any of which may be used in a respective sample cartridge. Each cartridge includes a filter material having a surface, in accordance with embodiments, is modified with a DNA binding ligand. Cartridge A performs only mechanical lysing for more hardy targets and includes a filter membrane modified with a DNA binding ligand. Cartridges B and C perform only chemical lysing for viruses, free NA or more fragile targets and include a filter column modified with a DNA binding ligand. The universal cartridge includes a modified filter (40), preferably a modified glass fiber filter and is sized to be secure between the valve cap and valve body.

FIG. 11B illustrates a universal valve assembly, in accordance with some embodiments, which utilizes glass beads son the modified glass fiber filter suited for mechanical lysis of certain types of targets, as compared to conventional assemblies in conventional sample cartridges. In this embodiment, the modified filter 40 is formed of glass fibers and has a 0.7 um pore size. In contrast, Cartridge A utilizes a filter formed as a disk of a polymer film (i.e., PCTE), which while suitable for mechanical lysing, but not suited for chemical lysing. By utilizing a filter having a pore size of 0.7 um, the filter is suitable for receiving suitably sized glass beads for mechanical lysing. Utilizing glass fibers to form the filter facilitates affinity bonding with the free nucleic acid released by chemical lysing. Thus, this filter is suited for both mechanical and chemical lysing.

FIG. 12 shows a comparison of glass fiber filters modified with 2% APTES by chemical vapor deposition (CVD1-4) compared to unmodified glass fiber filter (85A and RCC).

### DETAILED DESCRIPTION

### Definitions

To facilitate an understanding of the present disclosure, a number of terms and phrases are defined below:

As used in the specification and the appended claims, the singular forms “a,” “an” and “the” include plural referents unless the context clearly dictates otherwise. Thus, for example, reference to “a compound” includes mixtures of compounds, reference to “an amine group” includes mixtures of two or more such amine groups, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “alkyl” as used herein means an alkyl group, as defined above, but having from one to twenty carbons, more preferably from one to ten carbon atoms in its backbone structure. Likewise, “alkenyl” and “alkynyl” have similar chain lengths.

The alkyl groups can also contain one or more heteroatoms within the carbon backbone. Examples include oxygen, nitrogen, sulfur, and combinations thereof. In certain embodiments, the alkyl group contains between one and four heteroatoms.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

“Alkenyl” and “Alkynyl”, as used herein, refer to unsaturated aliphatic groups containing one or more double or triple bonds analogous in length (e.g., C₂-C₃₀) and possible substitution to the alkyl groups described above.

“Aryl”, as used herein, refers to 5-, 6- and 7-membered aromatic rings. The ring can be a carbocyclic, heterocyclic, fused carbocyclic, fused heterocyclic, bicarbocyclic, or biheterocyclic ring system, optionally substituted as described above for alkyl. Broadly defined, “Ar”, as used herein, includes 5-, 6- and 7-membered single-ring aromatic groups that can include from zero to four heteroatoms. Examples include, but are not limited to, benzene, pyrrole, furan, thiophene, imidazole, oxazole, thiazole, triazole, pyrazole, pyridine, pyrazine, pyridazine and pyrimidine. Those aryl groups having heteroatoms in the ring structure can also be referred to as “heteroaryl”, “aryl heterocycles”, or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic.

“Alkylaryl” or “aryl-alkyl”, as used herein, refers to an alkyl group substituted with an aryl group (e.g., an aromatic or hetero aromatic group).

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, containing carbon and one to four heteroatoms each selected from non-peroxide oxygen, sulfur, and N(Y) wherein Y is absent or is H, O, (C₁₋₄) alkyl, phenyl or benzyl, and optionally containing one or more double or triple bonds, and optionally substituted with one or more substituents. The term “heterocycle” also encompasses substituted and unsubstituted heteroaryl rings. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Heteroaryl”, as used herein, refers to a monocyclic aromatic ring containing five or six ring atoms containing carbon and 1, 2, 3, or 4 heteroatoms each selected from non-peroxide oxygen, sulfur, and N(Y) where Y is absent or is H, O, (C₁-C₈) alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula: —NR₉R₁₀ or NR₉R₁₀R′₁₀, wherein R₉, R₁₀, and R′₁₀ each independently represent a hydrogen, an alkyl, an alkenyl, —(CH₂)ₘ—R′₈ or R₉ and R₁₀ taken together with the N atom to which they are attached complete a heterocycle having from 4 to 8 atoms in the ring structure; R′8 represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In some embodiments, the term “amine” does not encompass amides, e.g., wherein one of R₉ and R₁₀ represents a carbonyl. In some embodiments, R₉ and R₁₀ (and optionally R′₁₀) each independently represent a hydrogen, an alkyl or cycloakly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The terms “amido” or “amide” is art-recognized as an amino-substituted carbonyl and includes a moiety that can be represented by the general formula —CONR₉R₁₀ wherein R₉ and R₁₀ are as defined above.

“Halogen”, as used herein, refers to fluorine, chlorine, bromine, or iodine.

“Hydroxyl”, as used herein, refers to —OH.

The term “carbonyl” is art-recognized and includes such moieties as can be represented by the general formula —CO—XR₁₁, or —X—CO—R′¹¹, wherein X is a bond or represents an oxygen or a sulfur, and R₁₁ represents a hydrogen, an alkyl, a cycloalkyl, an alkenyl, a cycloalkenyl, or an alkynyl, R′¹¹ represents a hydrogen, an alkyl, a cycloalkyl, an alkenyl, a cycloalkenyl, or an alkynyl. Where X is an oxygen and R₁₁ or R′¹¹ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′¹¹ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aryloxy, substituted aryloxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

As used herein, the terms “hydrolyzable” refers to a group or moiety which is capable of undergoing hydrolysis or solvolysis. For example, a hydrolyzable group can be hydrolyzed (i.e., converted to a hydrogen group) by exposure to water or a protic solvent at or near ambient temperature or an elevated temperature and at or near atmospheric pressure or an elevated pressure. In some cases, a hydrolyzable group can be hydrolyzed by exposure to acidic or alkaline water or acidic or alkaline protic solvent. Typical hydrolyzable groups include, but are not limited to, alkoxy, aryloxy, aralkyloxy, acyloxy, or halo. As used herein, the term is often used in reference to one of more groups bonded to a silicon atom in a silyl group.

As used herein, the terms “detect”, “detecting” or “detection” may describe either the general act of discovering or discerning or the specific observation of a detectably labeled composition.

The term “nucleic acid” refers to a nucleotide polymer, and unless otherwise limited, includes analogs of natural nucleotides that can function in a similar manner (e.g., hybridize) to naturally occurring nucleotides. The term nucleic acid includes any form of DNA or RNA, including, for example, genomic DNA; complementary DNA (cDNA), which is a DNA representation of mRNA, usually obtained by reverse transcription of messenger RNA (mRNA) or viral RNA or by amplification; DNA molecules produced synthetically or by amplification; mRNA; and non-coding RNA. The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like. More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs. The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the terms “oligonucleotide,” “polynucleotide,” and the like, refer to nucleic acid-containing molecules, including but not limited to, DNA or RNA. The term “oligonucleotide” is used to refer to a nucleic acid that is relatively short, generally shorter than 500 nucleotides, particularly, shorter than 200 nucleotides, more particularly, shorter than 100 nucleotides, most particularly, shorter than 50 nucleotides. Typically, oligonucleotides are single-stranded DNA molecules. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

In some embodiments, an oligonucleotide is 8 to 200, 8 to 100, 12 to 200, 12 to 100, 12 to 75, or 12 to 50 nucleotides long. Oligonucleotides may be referred to by their length, for example, a 24 residue oligonucleotide may be referred to as a “24-mer.”

The term “nucleic acid amplification,” encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase polymerase amplification and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), digital amplification, and the like. Descriptions of such techniques can be found in, among other sources, Ausbel et al.; PCR Primer: A Laboratory Manual, Diffenbach, Ed., Cold Spring Harbor Press (1995); U.S. Pat. Nos. 5,830,711, 6,027,889, and 5,686,243.

A “sample,” or “biological sample” as used herein, includes various nucleic acid (e.g., DNA and/or RNA) containing samples of tissue, cells, or fluid isolated from a subject, including but not limited to, for example, whole blood, buffy coat, plasma, serum, immune cells (e.g., monocytes or macrophages), and sputa. In some embodiments, the sample comprises a buffer, such as an anticoagulant, and/or a preservative. In some embodiments whole blood is mixed with heparin in a lithium heparin blood collection tube. The sample can be from any bodily fluid, tissue or cells that contain the expressed biomarker. A biological sample can be obtained from a subject by conventional techniques. For example, blood can be obtained by venipuncture or a finger-prick capillary, and solid tissue samples can be obtained by surgical techniques according to methods well known in the art. In some aspects the blood sample is placed into a tube that is specifically designed for the assay.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

As used herein, the term “detecting” refers to “determining the presence of” an item, such as a nucleic acid sequence, e.g., one that is indicative of the presence of a coronavirus. Detection can include the determination of the presence of a coronavirus, without definitive identification of that coronavirus; the determination of the presence of one or more coronaviruses belonging to a class of coronaviruses; the determination of the presence of a particular, known coronavirus strain; or determination of the presence of a novel (not previously described) coronavirus strain.

As used herein, “Clinical Laboratory Improvement Amendments (CLIA)” refers to The Clinical Laboratory Improvement Amendments of 1988 (CLIA) regulations in effect as of the original filing date of the present application. The CLIA regulations include federal standards applicable to all U.S. facilities or sites that test human specimens for health assessment or to diagnose, prevent, or treat disease. A “CLIA-compliant” test is one that complies with these regulations. “CLIA-waived” tests include tests that does not comply with all of these regulations. For example, CLIA-waived tests include test systems cleared by the U.S. Food and Drug Administration for home use and those tests approved for waiver under the CLIA criteria.

(a) Solid Support

Provided herein are solid supports for isolation and purification of nucleic acids from nucleic-acid containing samples, the solid support comprising a DNA binding ligand. In some aspects, the DNA binding ligand is chemically bonded to a surface of the solid support. For example, the DNA binding ligand can be covalently bonded (such as via a siloxane bridge) to the solid support. In other examples, the DNA binding ligand is chemically bonded to the solid support via a linker (such as by an oligoethylene linker or a PEG oligomer). In other aspects of the compositions and methods disclosed herein, the DNA binding ligand is not chemically bonded to the solid support.

As used herein, the term “solid support” refers to any substrate selected from paramagnetic particles, gels, fibers, controlled pore glass, magnetic beads, microspheres, nanospheres, capillaries, filter membranes, columns, cloths, wipes, paper, flat supports, multi-well plates, porous membranes, porous monoliths, wafers, combs, or any combination thereof. Solid supports can comprise any suitable material, including but not limited to glass, silica, titanium oxide, aluminum oxide, iron oxide, ethylenic backbone polymers, polypropylene, polyethylene, polystyrene, ceramic, cellulose, nitrocellulose, magnetic silica particles (such as MagneSil™ particles available from Promega Corporation), and divinylbenzene. In some embodiments, the solid support comprises a material selected from polystyrene, glass, ceramic, polypropylene, polyethylene, silica, mica, titanium dioxide, polycarbonate, latex, PMMA, zeolite, polyethersulfone, carboxymethylcellulose, cellulose, and combinations thereof. Examples of solid support includes a magnetic bead, a glass bead, a polystyrene bead, cellulose filter, a polystyrene filter, a polycarbonate filter, a polyethersulfone filter, polytetrafluoroethylene filter, polyvinylpyrrolidone filter, or a glass fiber filter. The solid support may further comprise a polymeric binder for binding the particles or fibers in the solid support. Exemplary polymeric binders include an acrylic polymer. In the disclosure provided herein, the solid support generally includes a surface functional groups that can interact and/or reactive with the DNA binding ligand (e.g., compounds having a silane group).

In some embodiments, the solid support is a fiber material, preferably, a glass fiber filter (GFF). Fibrous filters such as glass fiber filters offer several advantages over other porous supports such as glass beads. Porous glass fiber filters have much larger surface area than flat glass surfaces, but less than porous glass beads. Unlike glass beads, the thin, paper-like sheets of glass fiber filters are easily handled in aqueous or organic solvents. Mechanical stability of the glass fiber filters allows belts and sheets to be used in high throughput manufacturing. Unlike beads, glass fiber filter simplifies flow through filtering with no containing frits required. This feature of GFF allows construction of multilayer devices, where several modified glass fiber filters can be stacked on each other inside a cylindrical, flow through housing. If more DNA binding capacity is required, the effective thickness of the filters can be adjusted by stacking multiple discs.

The fiber material can be characterized by fiber diameter, pore diameter, basis weight, thickness, and/or specific surface area. The fibers in the fiber material can have an average diameter of 1 micron or greater, 1.5 microns or greater, 2 microns or greater, 2.5 microns or greater, 3 microns or greater, 3.5 microns or greater, 4 microns or greater, 4.5 microns or greater, 5 microns or greater, 5.5 microns or greater, 6 microns or greater, 6.5 microns or greater, 7 microns or greater, 7.5 microns or greater, 8 microns or greater, 9 microns or greater, 10 microns or greater, 12 microns or greater, 15 microns or greater, 16 microns or greater, 18 microns or greater, 19 microns or greater, or 20 microns or greater. In certain embodiments, the fibers in the fiber material can have an average diameter of 25 microns or less, 24 microns or less, 22 microns or less, 20 microns or less, 19 microns or less, 18 microns or less, 16 microns or less, 15 microns or less, 14 microns or less, 13 microns or less, 12 microns or less, 10 microns or less, 9 microns or less, 8 microns or less, 7.5 microns or less, 7 microns or less, 6 microns or less, 5.5 microns or less, or 5 microns or less. In certain embodiments, the fibers in the fiber material can have an average diameter from 1 micron to 20 microns, from 2 microns to 20 microns, from 2 microns to 18 microns, from 2.5 microns to 15 microns, from 2.5 microns to 12 microns, from 2.5 microns to 10 microns, from 3 microns to 20 microns, from 3 microns to 18 microns, from 3 microns to 15 microns, from 3 microns to 12 microns, from 4 microns to 20 microns, from 4 microns to 15 microns, or from 5 microns to 20 microns.

The fiber material can have an effective pore size (or average pore diameter) of 0.20 microns or greater, 0.30 microns or greater, 0.40 microns or greater, 0.50 microns or greater, 0.55 microns or greater, 0.60 microns or greater, 0.65 microns or greater, 0.70 microns or greater, 0.75 microns or greater, 0.80 microns or greater, 0.85 microns or greater, 0.90 microns or greater, 0.95 microns or greater, 1.0 microns or greater, or 1.1 microns or greater. In certain embodiments, the fiber material can have an average pore size of 3.0 microns or less, 2.0 microns or less, 1.9 microns or less, 1.8 microns or less, 1.7 microns or less, 1.6 microns or less, 1.5 microns or less, 1.4 microns or less, 1.3 microns or less, 1.2 microns or less, 1.1 microns or less, 1.05 microns or less, 1.0 microns or less, 0.95 microns or less, 0.90 microns or less, 0.85 microns or less, 0.80 microns or less, 0.75 microns or less, or 0.70 microns or less. In certain embodiments, the fiber material can have an average pore size from 0.2 μm to 3 μm, from 0.20 microns to 2.0 microns, from 0.20 microns to 1.5 microns, from 0.40 microns to 1.5 microns, from 0.40 microns to 1.3 microns, from 0.40 microns to 1.2 microns, from 0.50 microns to 1.5 microns, from 0.50 microns to 1.3 microns, from 0.50 microns to 1.2 microns, from 0.60 microns to 1.5 microns, from 0.60 microns to 1.3 microns, from 0.60 microns to 1.2 microns, from 0.70 microns to 1.5 microns, from 0.70 microns to 1.3 microns, from 0.70 microns to 1.2 microns, or from 0.70 microns to 1.0 micron.

The fiber material, such as the glass fiber filter can have a pore size selected to accommodate correspondingly sized beads to facilitate mechanical lysis. The beads can include glass beads, silica beads, or a combination thereof.

The thickness of the fiber material can be 100 microns or greater, 150 microns or greater, 200 microns or greater, 250 microns or greater, 300 microns or greater, 350 microns or greater, 400 microns or greater, 450 microns or greater, 500 microns or greater, 550 microns or greater, 600 microns or greater, 650 microns or greater, 700 microns or greater, 750 microns or greater, 800 microns or greater, 900 microns or greater, 1,000 microns or greater, 1,200 microns or greater, 1,500 microns or greater, 1,600 microns or greater, 1,800 microns or greater, 1,900 microns or greater, or 2,000 microns or greater. In certain embodiments, the fiber material can have a thickness of 2,500 microns or less, 2,400 microns or less, 2,200 microns or less, 2,000 microns or less, 1,900 microns or less, 1,800 microns or less, 1,600 microns or less, 1,500 microns or less, 1,400 microns or less, 1,300 microns or less, 1,200 microns or less, 1,000 microns or less, 900 microns or less, 800 microns or less, 750 microns or less, 700 microns or less, 600 microns or less, 550 microns or less, or 500 microns or less. In certain embodiments, the fiber material can have a thickness from 100 microns to 2,000 microns, from 200 microns to 1,500 microns, from 200 microns to 1,200 microns, from 250 microns to 1,200 microns, from 250 microns to 1,000 microns, from 250 microns to 800 microns, from 300 microns to 2,000 microns, from 300 microns to 1,800 microns, from 300 microns to 1,500 microns, from 300 microns to 1,200 microns, from 400 microns to 2,000 microns, from 400 microns to 1,500 microns, or from 500 microns to 2,000 microns.

The basis weight of the fiber material can be 10 g/m² or greater, 15 g/m² or greater, 20 g/m² or greater, 25 g/m² or greater, 30 g/m² or greater, 35 g/m² or greater, 40 g/m² or greater, 45 g/m² or greater, 50 g/m² or greater, 55 g/m² or greater, 60 g/m² or greater, 65 g/m² or greater, 70 g/m² or greater, 75 g/m² or greater, 80 g/m² or greater, 90 g/m² or greater, 100 g/m² or greater, 120 g/m² or greater, 150 g/m² or greater, 160 g/m² or greater, 180 g/m² or greater, 190 g/m² or greater, or 200 g/m² or greater. In certain embodiments, the fiber material can have a basis weight of 250 g/m² or less, 240 g/m² or less, 220 g/m² or less, 200 g/m² or less, 190 g/m² or less, 180 g/m² or less, 160 g/m² or less, 150 g/m² or less, 140 g/m² or less, 130 g/m² or less, 120 g/m² or less, 100 g/m² or less, 90 g/m² or less, 80 g/m² or less, 75 g/m² or less, 70 g/m² or less, 60 g/m² or less, 55 g/m² or less, or 50 g/m² or less. In certain embodiments, the fiber material can have a basis weight from 10 g/m² to 200 g/m², from 20 g/m² to 150 g/m², from 20 g/m² to 120 g/m², from 25 g/m² to 120 g/m², from 25 g/m² to 100 g/m², from 25 g/m² to 90 g/m², from 30 g/m² to 200 g/m², from 30 g/m² to 180 g/m², from 30 g/m² to 150 g/m², from 30 g/m² to 100 g/m², from 40 g/m² to 150 g/m², from 40 g/m² to 100 g/m², or from 50 g/m² to 90 g/m².

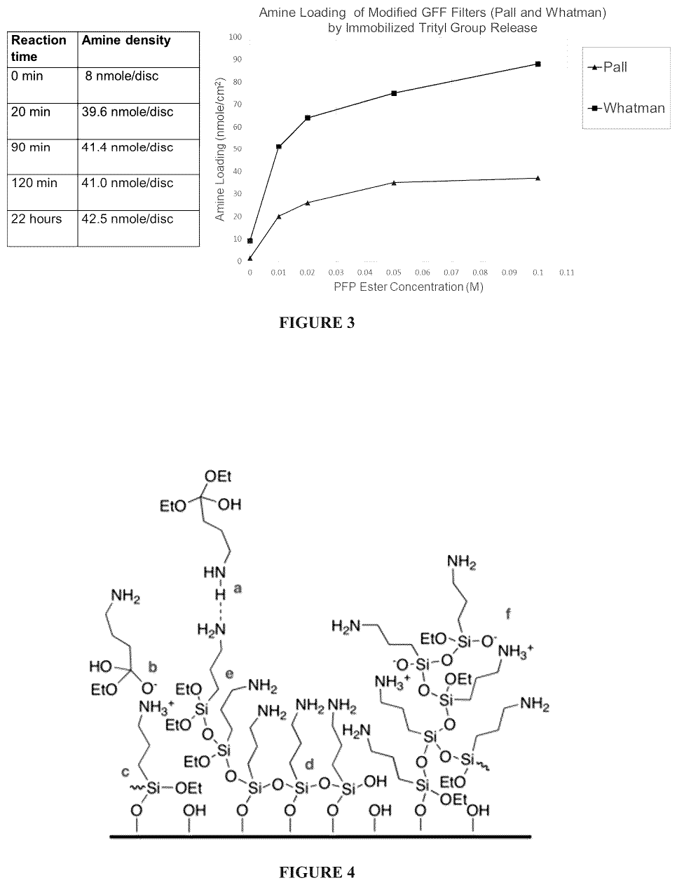

In some examples, unmodified solid support (which can be modified to include a DNA binding ligand) can be obtained from Pall Corporation, having different pore sizes and thickness: For example, glass fiber filters are available from Pall Corporation as Type A/E, A/B and A/C, all have 1 μm nominal pore size with thickness of 0.33 mm, 0.66 mm and 0.25 mm respectively. Others filter types are described below. The Pall TCLP (Toxic Characteristics Leaching Procedure) product has the same dimensions as the Whatman (Cytiva) filters described in FIG. 3 (0.7 μm pore size, 0.4 mm thick).

TABLE A
Specifications of Exemplary Glass Fiber
Filters available from Pall Corporation

| | | Extra Thick | Metrigard ™ | |
|---|---|---|---|---|
| Description | Type A/D | Discs | Discs | TCLP |
| Typical | Prefiltration | Prefiltration | Prefiltration | U.S. EPA |
| Applications | of solutions | of heavily | in systems with | Method |
| with large- | | contaminated | high particulate | 1311 |
| sized | | samples | matter | |
| particulate | | | | |
| Filter Media | Borosilicate | Glass fiber | Ultrafine glass | Borosilicate |
| | glass without | with acrylic | fiber with | glass without |
| | binder | binder | acrylic binder | binder |
| Pore Size | 3 μm | 1 μm | 0.5 μm | 0.7 μm |
| (Nominal) | | | | |
| Typical | 660 μm (26 | 1270 μm (50 | 330 μm (13 | 432 μm (17 |
| Thickness | mils) | mils) | mils) | mils) |

In specific embodiments, the solid support is a glass fiber filter having a thickness of from 400 microns to 2000 microns and a pore size of 0.5 microns to 1 micron.

In other embodiments, the solid support is particulate material, such as silica gel or silica having a pore diameter from about 30 to about 1000 Angstroms, a particle size from about 2 to about 300 microns, and a specific surface area from about 35 m²/g to about 1000 m²/g. In some embodiments, particulate material can have a pore diameter of about 40 Angstroms to about 500 Angstroms, about 60 Angstroms to about 500 Angstroms, about 100 Angstroms to about 300 Angstroms, and about 150 Angstroms to about 500 Angstroms. In some embodiments, the particulate material can have a particle size of about 2 to about 25 microns, about 5 to about 25 microns, about 15 microns, about 63 to about 200 microns, and about 75 to about 200 microns; and a specific surface area of about 100 m²/g to about 350 m²/g, about 100 m²/g to about 500 m²/g, about 65 m²/g to about 550 m²/g, about 100 m²/g to about 675 m²/g, and about 35 to about 750 m²/g.

As described herein, the solid support comprises a DNA binding ligand such as an amino-containing compound and can be used as a separating material for nucleic acid isolation. Particularly, the DNA binding ligand on the surface of the solid support provides high nucleic acid binding capacity for isolating the nucleic acid from a sample. Accordingly, disclosed herein are separating materials for nucleic acid isolation comprising a solid support (e.g., a glass fiber solid support) comprising a DNA binding ligand. In some embodiments, the separating material for nucleic acid isolation is selected from columns, capillaries, or cartridges containing a modified solid support as disclosed herein. In some embodiments, the separating materials are useful for isolation, separation, and purification of nucleic acids, for example, from a biological sample or a chemical reaction mixture.

The separating material comprising the solid support can have a surface density of the DNA binding ligand (such as an alkylamine compound) of 10 nmoles of compound/cm² or greater (for e.g., 15 nmoles/cm² or greater, 20 nmoles/cm² or greater, 25 nmoles/cm² or greater 35 nmoles/cm² or greater, 40 nmoles/cm² or greater, 45 nmoles/cm² or greater, 50 nmoles/cm² or greater, 55 nmoles/cm² or greater, 60 nmoles/cm² or greater, 65 nmoles/cm² or greater, 70 nmoles/cm² or greater, 75 nmoles/cm² or greater, 80 nmoles/cm² or greater, 85 nmoles/cm² or greater, 90 nmoles/cm² or greater, 95 nmoles/cm² or greater, 100 nmoles/cm² or greater, from 10-100 nmoles/cm², from 15-80 nmoles/cm², from 30-100 nmoles/cm² or from 30-90 nmoles/cm²). The high density of the DNA binding ligand on the surface of the solid support provides high nucleic acid binding capacity for isolating the nucleic acid from a sample. An assay for measuring density of surface amine or amide groups on the solid support (such as GFF) is also disclosed herein.

The separating material comprising the solid support can have a DNA binding capacity of at least 10 μg/cm² (for e.g., 15 μg/cm² or greater, 20 μg/cm² or greater, 25 μg/cm² or greater, 35 μg/cm² or greater, 40 μg/cm² or greater, 45 μg/cm² or greater, 50 μg/cm² or greater, 55 μg/cm² or greater, 60 μg/cm² or greater, 65 μg/cm² or greater, 70 μg/cm² or greater, 75 μg/cm² or greater, 80 μg/cm² or greater, 85 μg/cm² or greater, 90 μg/cm² or greater, 95 μg/cm² or greater, 100 μg/cm² or greater, from 30-100 μg/cm² or from 30-90 μg/cm²). Of course, separating materials comprising the solid support with higher DNA binding capacity are preferred, such have a DNA binding capacity of at least 30 μg/cm².

Compounds

As described herein, the solid support comprises a DNA binding ligand. The term “DNA binding ligand” is used extensively herein. However other types of nucleic acids other than DNA are relevant. Consequently, it is intended that in general the above term can be replaced with the terms “nucleic acid binding ligand” or “nucleic acid binding molecule”. Nucleic acids will in general be RNA or DNA, double stranded or single stranded, or having secondary (single stranded DNA or RNA can form internal double stranded regions, i.e., secondary structures) or tertiary structures.

The DNA binding ligand includes any molecule which is capable of binding or associating with DNA. This binding or association may be via covalent bonding, via ionic bonding, via hydrogen bonding, via Van-der-Waals bonding, or via any other type of reversible or irreversible association. The term “ligand” is used herein to refer to any atom, ion, molecule, macromolecule (for example polypeptide), or combination of such entities. The term “ligand” is used interchangeably with the term “molecule”.

The DNA binding ligand can be selected from an amine containing compound such as an alkylamine, a cycloalkylamine, an alkyloxy amine, a polyamine, or an arylamine), an intercalating agent (e.g., furocoumarins, coumarins, anthracyclines, phenanthridines, psoralen derivatives, acridines, ellipticines, actinomycins, anthracenediones, and Tris compounds), a groove binder (e.g., pyrrolo(1,4)benzodiazepines (PBD's), anthelvencins, kikumycins, netropsin, distamycin, calicheamicin, CC-1065, and Hoechst 33258), a polypeptide, an amino acid (histidine), a protein (such as zinc finger proteins, homeodomains, leucine zipper proteins, helix-loop-helix proteins or β-sheet motifs), or a combination thereof.

The solid support described herein can comprise a compound derived from a structure represented by the formula:

Y-(L)_{y}-SiX₃
wherein,
Y is a DNA binding ligand selected from an alkylamine, a cycloalkylamine, an alkyloxy amine, a polyamine moiety, an intercalating agent, a minor groove binder, a peptide, an amino acid, an arylamine, or a combination thereof,
L is a linker selected from an alkyl group, a heteroalkyl group, an alkene group, a heteroalkene group, a polyacrylic acid, a Diels-Alder adduct, or a combination thereof,
each X, independently for each occurrence, is selected from a hydrolyzable group, an alkyl group, a heteroalkyl group, an alkenyl group, or two or three Xs combine to form one or more cyclic groups, or one X combines with Y to form a cyclic azasilane, and
y is 0 or 1.

In some aspects of the compounds disclosed herein, the DNA binding ligand or Y can comprise a plurality of amine groups (or a polyamine), a plurality of amide groups (or a polyamide), or a combination thereof (a polyamine-amide). For example, the DNA binding ligand or Y can comprise at least two, at least three, at least four, at least five, at least six amine or amide groups, or a combination thereof. In other examples, the DNA binding ligand or Y comprises a single amine or amide group. The amine or amine group can be a primary, secondary, or tertiary amine. In some embodiments, the DNA binding ligand or Y comprises a quaternary ammonium group.

In some aspects of the compounds, the DNA binding ligand or Y comprises a C₁-C₁₆ alkylamine (e.g., C₁-C₁₂ alkylamine, C₁-C₁₀ alkylamine, C₁-C₈ alkylamine, C₂-C₈ alkylamine, or C₂-C₆ alkylamine), a C₃-C₁₂ cycloalkylamine (e.g., C₃-C₁₀ cycloalkylamine, C₃-C₈ cycloalkylamine, C₃-C₆ cycloalkylamine, C₄-C₈ cycloalkylamine, or C₄-C₆ cycloalkylamine), an C₁-C₁₆ alkyloxy amine (e.g., C₁-C₁₂ alkyloxy amine, C₁-C₁₀ alkyloxy amine, C₁-C₈ alkyloxy amine, C₂-C₈ alkyloxy amine, or C₂-C₆ alkyloxy amine), a C₆-C₁₂ arylamine (e.g., C₆-C₁₀ arylamine, C₆-C₈ arylamine), a C₆-C₁₂ imidazole group, a C₃-C₁₄ hetero cycloalkylamine (e.g., C₃-C₁₀ hetero cycloalkylamine, C₃-C₈ hetero cycloalkylamine, C₃-C₆ hetero cycloalkylamine, C₄-C₈ hetero cycloalkylamine, or C₄-C₆ hetero cycloalkylamine), and a C₂-C₂₀ heteroalkylamine (e.g., C₁-C₁₂ heteroalkylamine, C₁-C₁₀ heteroalkylamine, C₁-C₈ heteroalkylamine, C₂-C₈ heteroalkylamine, or C₂-C₆ heteroalkylamine), or a combination thereof. In some embodiments, the DNA binding ligand or Y comprises an alkylamine group, an imidazole group, or a combination thereof.

In some examples of the compounds, the DNA binding ligand or Y is selected from spermidine, spermine, methylamine, ethylamine, propylamine, cadaverine, putrescine, ethylenediamine, diethylene triamine, 1,3-dimethyldipropylenediamine, 3-(2-aminoethyl)aminopropyl, (2-aminoethyl)trimethylammonium hydrochloride, tris(2-aminoethyl)amine, or a combination thereof. The DNA binding ligand or Y is optionally substituted with one or more groups, such as a C₁-C₆ alkyl, a heteroalkyl, or an amino group.

In some aspects of the compounds disclosed herein, the linker, L, is present, that is, y is 1. The linker can be selected from an alkyleneoxy (e.g., a C₂-C₄ alkyleneoxy) group, an alkylene (e.g., a C₂-C₄ alkylene or C₂-C₃ alkylene) group, or a heteroalkylene (e.g., C₄-C₆ heteroalkylene). In some examples, L is a bond. In other examples, L can be derived from a Diels-Alder adduct. The Diels-Alder adduct can be derived from an unsaturated cyclic imido group.

In some aspects of the compounds disclosed herein, each X can be independently selected from a halogen (such as Cl, Br, I,), a C₁-C₆ alkoxy, a dialkylamino, a trifluoromethanesulfonate, or a C₁-C₆ straight, branched, or cyclic alkyl. Preferably, at least two Xs include a hydrolysable group independently selected from a halogen, an alkoxy, a dialkylamino, a trifluoromethanesulfonate, or they combine together with the Si atom to which they are attached to form a silatrane, a cyclic siloxane, a polysilsesquioxane, or a silazane. In some examples, two Xs can be independently selected from a halogen (such as Cl, Br, I,), a C₁-C₆ alkoxy (such as ethoxy, methoxy, acetoxy), a dialkylamino, or a trifluoromethanesulfonate, and one X selected from a C₁-C₆ straight, branched, or cyclic alkyl.

Also disclosed herein are compositions comprising a Diels-Alder adduct, wherein the Diels-Alder adduct includes a DNA binding ligand. As defined herein, the DNA binding ligand can comprise an amine group, an intercalating agent, a minor groove binder, a peptide, an amino acid, a protein, or a combination thereof. In some examples, the DNA binding ligand is selected from an alkylamine, a cycloalkylamine, an alkyloxy amine, an arylamine, a polyamine moiety, or a combination thereof. The Diels-Alder adduct can be represented by the general Formula,

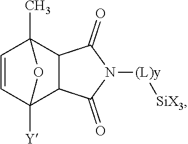

their isomers, salts, tautomers, or combinations thereof, and wherein Y′ is a DNA binding ligand as defined herein, and L, X, and y are also as defined herein. The Diels-Alder adduct can be covalently or noncovalently associated with the solid-support.

Specific examples of the compounds and compositions comprising the DNA binding ligand described here can include amino silanizing compounds selected from:

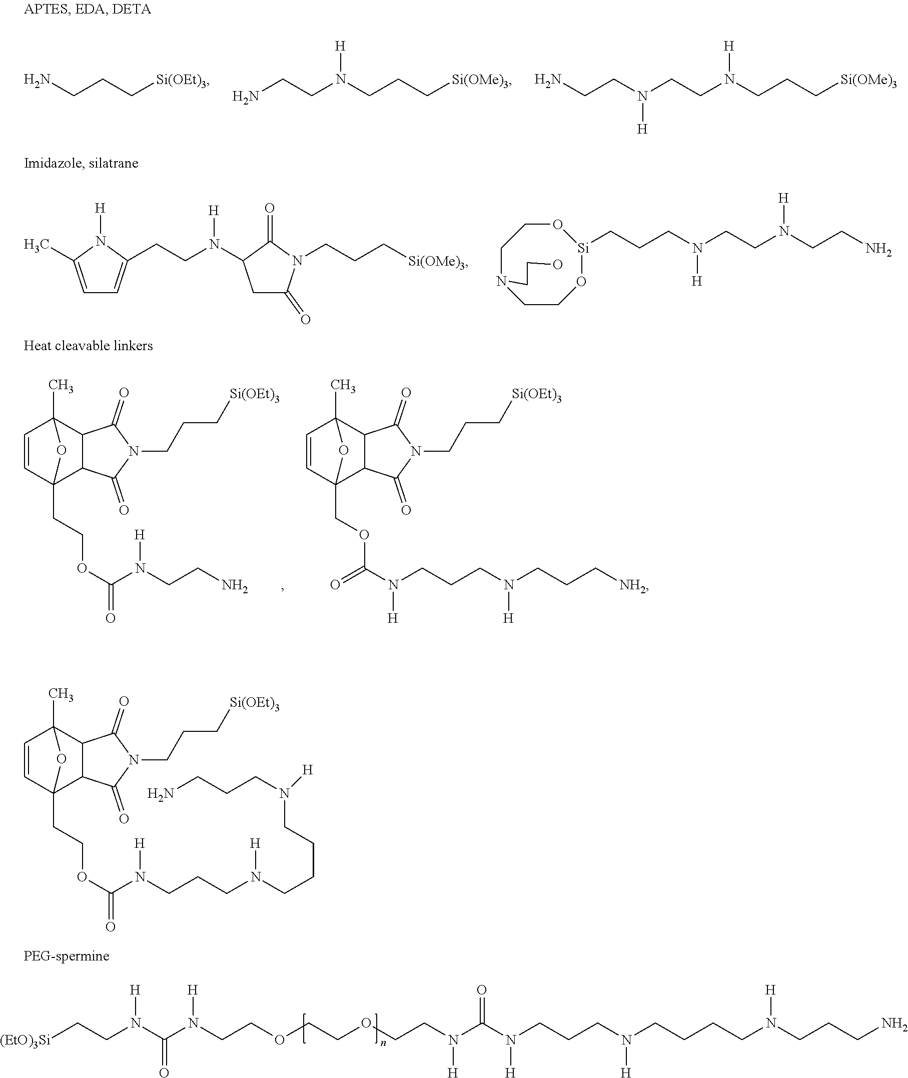

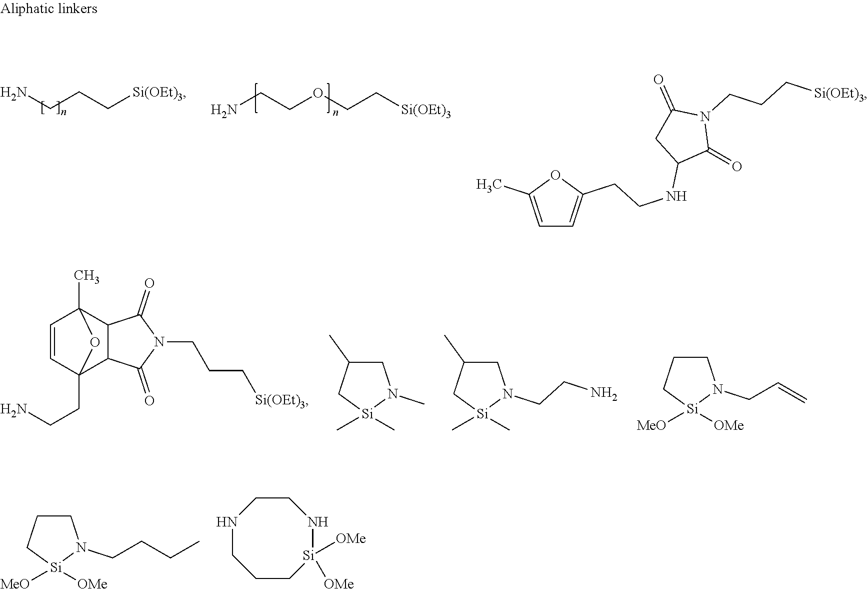

3-aminopropyltrimethoxysilane, 3-aminopropyltriethoxysilane, an aminoalkylsilatrane, 3-(2-aminoethyl)aminopropyltriethoxysilane, 3-(2-aminoethyl)aminopropyltrimethoxysilane, or a combination thereof, and wherein n is an integer from 0 to 10, from 1 to 10, or from 1 to 5.

In some instances, the compound can comprise a functional group such as a silanizing group or a moiety other than the silanizing group, that can facilitate binding with the solid support. For example, the compound can comprise an ether, a silyl ether, a siloxane, an ester of carboxylic acid, an ester of sulfonic acid, an esters of sulfamic acid, an ester of sulfuric acid, an ester of phosphonic acid, an ester of phosphinic acid, an ester of phosphoric acid, a silyl ester of carboxylic acid, a silyl ester of sulfonic acid, a silyl ester of sulfinic acid, a silyl ester of sulfuric acid, a silyl ester of phosphonic acid, a silyl ester of phosphinic acid, a silyl ester of phosphoric acid, an oxides, a sulfide, a carbocycle, a heterocycle with at least one oxygen atom, a heterocycle with at least one nitrogen atom, a heterocycle with at least one sulfur atom, a heterocycle with at least one silicon atom, a carbodiimide (such as DCC and EDCI), a phosphonium or imonium (such as BOP, PyBOP, PyBrOP, TBTU, HBTU, HATU, COMU, and TFFH), a ‘click’ reaction-derived heterocycle, a Diels-Alder reaction-derived carbocycle, a Diels-Alder reaction-derived heterocycle, an amide, an imide, a sulfide, a thiolate, a metal thiolate, a urethane, an oxime, a hydrazide, a hydrazone, a physisorbed or chemisorbed or otherwise non-covalently attached moiety, or a combination thereof. In certain embodiments, the compound includes a functional group selected from a maleimide, an acrylate, an acrylamide, an epoxide, an aziridine, a thiirane, an aldehyde, a ketone, an azide, an alkyne, a disulfide, an anhydride, a carboxylates phosphate, a phosphonate, a sulfate, a sulfonate, a nitrate, an amidine, a silane, a siloxane, a cyanate, an acetylene, a cyanide, a halogen, an acetal, a ketal, an amino, carbonyl, a carboxyl, biotin, cyclodextrin, an adamantane, or a vinyl group that can be attached to the solid support. In some embodiments, the solid support is glass comprising silanol and siloxane groups. Such silanol groups on the glass surface can be reacted with silane- and siloxane-containing compounds to provide a surface having a compound chemically bonded via a siloxane bridge. Methods of covalently linking compounds containing amino groups to functionalized surfaces and solid surfaces are known in the art.

Cartridges

In some embodiments, the solid support is incorporated into an automated cartridge, such as a GenXpert® cartridge. In one aspect, the invention pertains to a sample cartridge that utilizes a valve body platform that allows for detection of enveloped and free nucleic acid targets. In some embodiments, the valve body includes a sample processing region or lysing chamber that provides for either or both mechanical and chemical lysis. This allows a single cartridge to provide lysing for a multitude of differing types of targets, thus, can be considered an “assay panel cartridge.”

The sample cartridge can be any device configured to perform one or more process steps relating to preparation and/or analysis of a biological fluid sample according to any of the methods described herein. In some embodiments, the sample cartridge is configured to perform at least sample preparation. The sample cartridge can further be configured to perform additional processes, such as detection of a target nucleic acid in a nucleic acid amplification test (NAAT), e.g., Polymerase Chain Reaction (PCR) assay, by use of a reaction vessel attached to the cartridge. In some embodiments, the reaction vessel extends from the body of the sample cartridge. Preparation of a fluid sample generally involves a series of processing steps, which can include chemical, electrical, mechanical, thermal, optical or acoustical processing steps according to a specific protocol. Such steps can be used to perform various sample preparation functions, such as cell capture, cell lysis, binding of analyte, and binding of unwanted material.

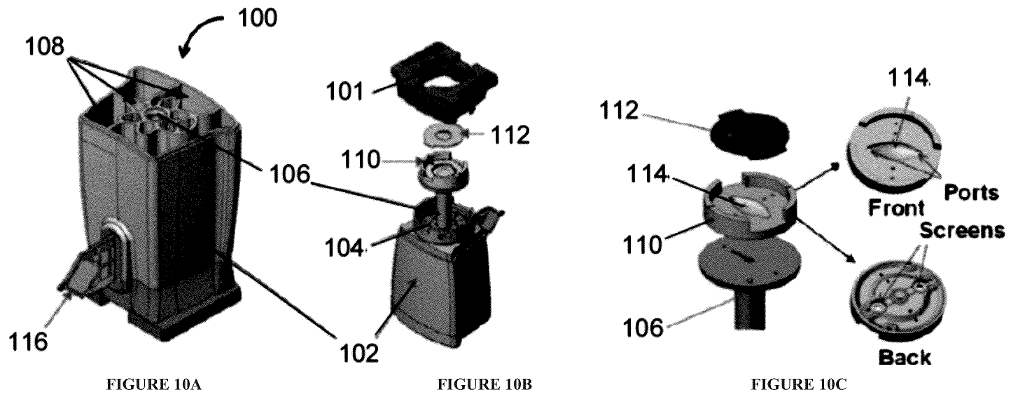

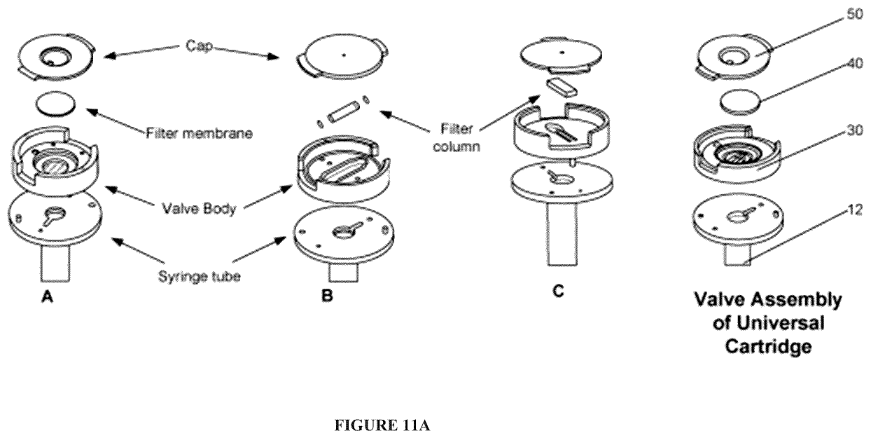

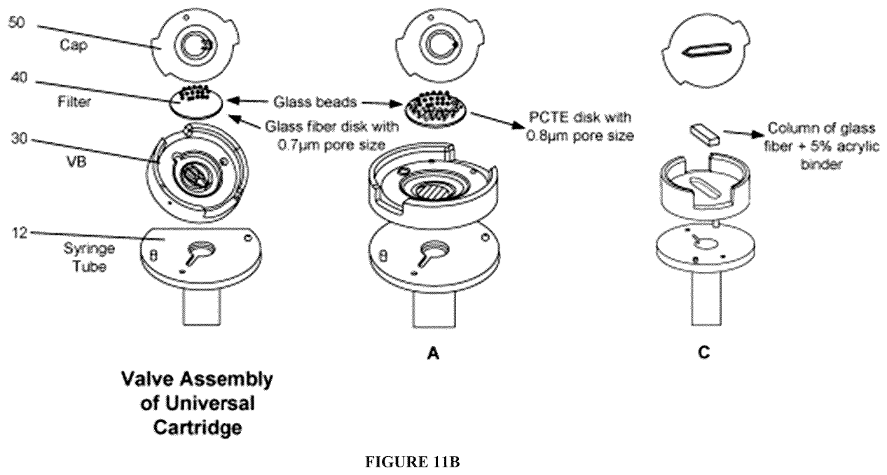

A cartridge suitable for use with the invention, includes one or more transfer ports through which the prepared fluid sample can be transported into an attached reaction vessel for analysis. FIG. 10A illustrates an exemplary assay panel cartridge 100 suitable for sample preparation and analytics testing by PCR when received in an instrument module in accordance with some embodiments. The sample cartridge is attached with a reaction vessel 116 (also referred to as a “reaction tube” or “PCR tube”) adapted for analysis of a fluid sample processed within the sample cartridge 100. In some embodiments the reaction vessel extends from the cartridge body. Such a sample cartridge 100 includes various components including a main housing 102 having one or more chambers 108 for processing of the fluid sample, which typically include sample preparation before analysis. The instrument module facilitates the processing steps needed to perform sample preparation and the prepared sample is transported through one of a pair of transfer ports into fluid conduit of the reaction vessel 116 attached to the housing of the sample cartridge 100. The prepared biological fluid sample is then transported into a reaction chamber of the reaction vessel where the biological fluid sample undergoes nucleic acid amplification. In some embodiments, the amplification is a polymerase chain reaction. In some embodiments, concurrent with the amplification of the biological fluid sample, an excitation means, and an optical detection means of the module is used to detect optical emissions that indicate the presence or absence of a target nucleic acid analyte of interest, e.g., a bacteria, a virus, a pathogen, a toxin, a tumor, or other target analyte. It is appreciated that such a reaction vessel could include various differing chambers, conduits, or micro-well arrays for use in detecting the target analyte. The cartridge is provided with means to perform preparation of the biological fluid sample before transport into the reaction vessel. Any chemical reagent required for cell lysis, or means for binding or detecting an analyte of interest (e.g., reagent beads) can be contained within one or more chambers of the cartridge, and as such can be used for sample preparation. U.S. Provisional Application No. 63/217,672 filed Jul. 1, 2021, further details the assay cartridge of valve assembly D and is incorporated herein by reference in its entirety.

An exemplary use of a reaction vessel for analyzing a biological fluid sample is described in commonly assigned U.S. Pat. No. 6,818,185, entitled “Cartridge for Conducting a Chemical Reaction,” filed May 30, 2000, the entire contents of which are incorporated herein by reference for all purposes. Examples of the sample cartridge and associated modules are shown and described in U.S. Pat. No. 6,374,684, entitled “Fluid Control and Processing System” filed Aug. 25, 2000, and U.S. Pat. No. 8,048,386, entitled “Fluid Processing and Control,” filed Feb. 25, 2002, U.S. Patent Application No. 63/218,672 entitled “Universal Assay Cartridge and Methods of Use” filed Jul. 1, 2021; U.S. Provisional Application No. 63/319,993 entitled “Unitary Cartridge Body and Associated Components and Methods of Manufacture” filed Mar. 15, 2022; and U.S. Pat. No. 10,562,030 entitled “Molecular Diagnostic Assay System” filed Jul. 22, 2016; the entire contents of which are incorporated herein by reference in their entirety for all purposes.

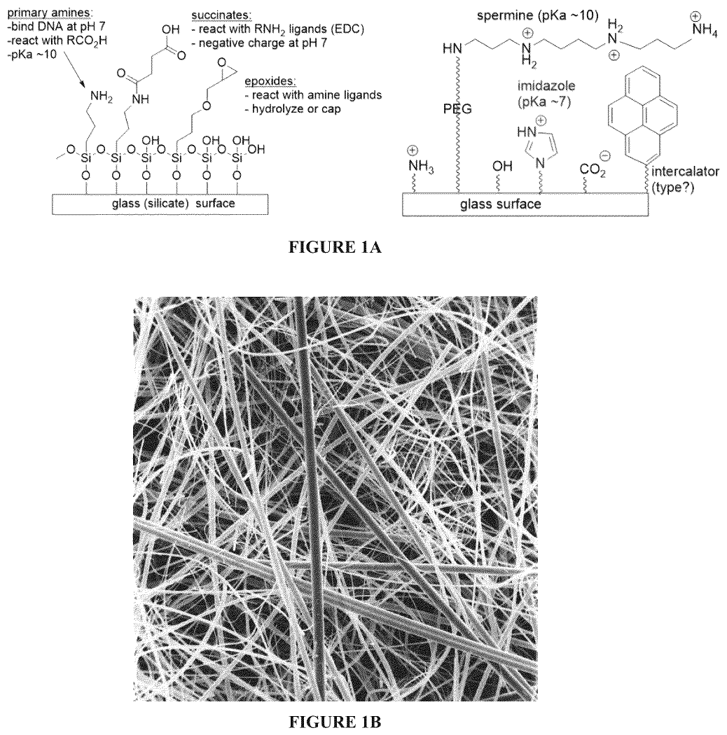

As shown in FIG. 10A, the assay panel cartridge 100 comprises a cartridge body 102 containing a plurality of chambers 108 for reagents or buffers and sample processing. The chambers are disposed around a central syringe barrel 106 that is in fluid communication with a valve body 110 (see FIGS. 1B and 10C) and that is sealed with a gasket 104. The valve body 110 can include a cap 112 and the entire cartridge body can be supported on a cartridge base 101. The valve body typically contains one or channels or cavities (chamber(s) 114) that can contain the filter or separating material described herein that can function to bind and elute a nucleic acid. In some embodiments the cartridge further comprises one or more temperature-controlled channels or chambers that can, in certain embodiments, function as thermocycling chambers. A “plunger” not shown can be operated to draw fluid into the syringe barrel 106 and rotation of the valve body 110 provides selective fluid communication between the various reagent chambers 108 and channels, reaction chamber(s), mixing chambers, and optionally, any temperature-controlled regions. Thus, the various reagent chambers 108, reaction chambers, matrix material(s), and temperature-controlled chambers or channels are selectively in fluid communication by rotation of the plunger and reagent movement (e.g., chamber loading or unloading) is operated by the “syringe” action of the plunger within the valve assembly.

FIGS. 11A and 11B illustrate differing valve assembles that can be used in the sample cartridge of FIG. 10A. Valve assembly A performs only mechanical lysing and is suitable for lysing hardy targets (e.g., certain bacteria, spores). Valve assemblies B and C perform only chemical lysing and is suitable for lysing less hardy targets (e.g., viruses, free NA, some spores, some bacteria and yeasts). The universal valve assembly can perform both mechanical and chemical lysing for all types of targets. In all such cartridges, the valve assembly includes the syringe tube, valve body (VB), and valve cap. The capabilities of the valve assembly of the sample cartridge rely on the filter or separating material described herein, as well as the particular workflow sequence performed by the instrument interface of the module. For example, valve assembly A has a valve body shaped with a circular cavity to support a filter disc to filter the sample, and the cap has a sonication dome feature, which interfaces with a sonication horn of a cartridge receiving module so as to ultrasonically lyse the target. By contrast, valve assemblies B and C have a valve body with an oblong filter recess that receives a filter (e.g., glass fiber filter column) therein, the filter configured for binding with nucleic acid released from the target optionally by chemical lysing. The universal valve assembly has a design more similar to valve assembly A, having a cap with a sonication dome, and a valve body with a circular cavity for supporting a disc filter, however this design uses a glass fiber filter as described herein. Utilizing the DNA binding ligands with the filter material (such as modified glass fibers) to form the filter facilitates affinity bonding with the free nucleic acid released from the biological sample, optionally by chemical lysing.

While the methods described herein are described primarily with reference to the GENEXPERT® cartridge by Cepheid Inc. (Sunnyvale, Calif) (see, e.g., FIG. 10A), it will be recognized, that in view of the teachings provided herein the methods can be implemented on other cartridge/microfluidic systems, including alternative cartridge designs having valve assemblies that involve multiple interfacing components, as well as cartridge body defined by multiple interfacing components to form the multiple chambers of the cartridges, for example, those described in Korean Application No. 102293717B1, cartridges that utilizes ultrasonic waves to lyse cells in a biological sample, for example, those described in International Application No. WO2021/245390A1, cartridges and systems that utilizes an electrowetting grid for microdroplet manipulation and electrosensor arrays configured to detect analytes of interest, for example, those described in International Application No. WO2016/077341A2, cartridges that facilitate movement of nucleic acid from one chamber to the next chamber by opening a vent pocket, for example, those described in International Application No. WO2012/145730A2, multiplexed assay systems comprising a plurality of thermocycling units such that individual chambers can be heated, cooled, and/or compressed to mix fluid within the chamber or to propel fluid in the chamber into another chamber, for example, those described in International Application No. WO2015/138343A1, and as well as systems for rapid amplification of nucleic acids facilitated by flexible portions of the sample cartridge aligned to accomplish temperature cycling for nucleic acid amplification, for example, those described in International Application No. WO2017/147085A1. Such cartridge/microfluidic systems can include, for example microfluidic systems implemented using soft lithography, micro/nano-fabricated microfluidic systems implemented using hard lithography, and the like. Additionally, it is appreciated that the assay panel methods described herein (i.e., identification of multiple conditions based on comparative levels of multiple-targets obtained from a single sample) can further be realized in entirely different systems, including: isothermal nucleic acid amplification systems, Digital RT-PCR, Electrochemical PCR, lateral flow testing cartridges, electrochemical sensors, nucleic acid sequencing, CRISPR/Cas BASED technologies, chemiluminescence, and nanoparticle-based colorimetric detection.

In some embodiments, the sample cartridge can comprise a) a cartridge body having a plurality of chambers defined therein, wherein the plurality of chambers are in in fluidic communication through a fluidic path of the cartridge, and wherein at least one chamber is configured to receive the biological sample, b) a reaction vessel configured for amplification of the nucleic acid by thermal cycling, and c) a filter disposed in the fluidic path between the plurality of chambers and the reaction vessel, wherein the filter comprises a separating material as disclosed herein, wherein the plurality of chambers and the reaction vessel independently comprise reagents for releasing nucleic acid from the biological sample, and primers and probes for detection of the nucleic acid.

In some embodiments, the sample cartridge can comprise a) a cartridge body having a plurality of chambers therein, wherein the plurality of chambers include: a sample chamber having at least a fluid outlet in fluid communication with another chamber of the plurality; and a lysis chamber in fluidic communication with the sample chamber, the lysis chamber comprising reagents for releasing nucleic acid, optionally wherein the sample chamber and lysis chamber are the same; b) a reaction vessel fluidically coupled to the plurality of chambers of the cartridge body and configured for amplification of nucleic acid by thermal cycling; c) a filter disposed in the fluidic path between the lysis chamber and the reaction vessel, wherein the filter comprises a solid support modified with a DNA binding ligand selected from an alkylamine, a cycloalkylamine, an alkyloxy amine, a polyamine moiety, an intercalating agent (e.g., tris compounds), a minor groove binder, a peptide, an amino acid (histidine), an arylamine, or a combination thereof, and d) a plurality of primers and/or probes disposed in one or more chambers of the plurality of chambers or reaction vessel for detection of the nucleic acid. The compound used to modify the filter can be as described herein.

The lysis chamber optionally comprises lysis reagents, the lysis reagents selected from a chaotropic agent, a chelating agent, a buffer, and a detergent. The lysis chamber may further comprise a valve body and a valve cap, wherein the valve body interfaces with the valve cap to define the lysis chamber therebetween, and wherein the filter is held within the lysis chamber secured between the valve body and the valve cap.

The lysis chamber has a fluid flow path between an inlet in the cap and an outlet in the valve body that is fluidically coupled to a fluid displacement region of the valve body, wherein the fluid displacement region is depressurizable by movement of the syringe to draw fluid into the fluid displacement region and pressurizable by movement of the syringe to expel fluid from the fluid displacement region. The sample cartridge together with the reagents can allow for flow rates up to about 100 μL per second, such as from about 10 μL to about 100 μL. The sample cartridge together with the reagents can allow for pressure below 100 psi, below 80 psi, or below 60 psi. The sample cartridge can allow for sample volumes up to 1000 μL, such as from 300 μL to 1,000 μL.

The cartridge body can further comprise an ultrasonic, piezoelectric, magnetostrictive, or electrostatic transducer, for example in the lysis chamber to facilitate mechanical lysing. The sample cartridge may further comprise a syringe that is movable to facilitate fluid flow into and from the lysis chamber by fluctuation of pressure.

The cartridge can be a single-use disposable cartridge. In some embodiments, the cartridge is an automated cartridge.

In order to increase sensitivity of detection, large sample volumes can be prepared. As described herein, the preparation of large volumes, however, is contradictory to microfluidic systems for automatic lysis, processing and/or analysis of biological samples. There is therefore a demand for solutions which permit preparing a large sample volume by means of, for example, filtration, and to make the isolated nucleic acids available in a small volume to a microfluidic system via a microfluidic interface. The sample cartridges comprising a modified filter, preferably a modified glass fiber filter and methods described herein are used for accomplishing this need. The cartridges and methods allow for the detection of target nucleic acid (e.g., DNA) from various sample types (including whole blood, plasma, serum, semen, spinal fluid, tissue, tear, urine, stool, saliva, respiratory sample, nasopharyngeal sample, vaginal swab, vaginal mucus sample, vaginal tissue sample, vaginal cell sample, bacterial culture, mammalian cell culture, viral culture, human cell, bacteria, extracellular fluid, pancreatic fluid, cell lysate, PCR reaction mixture, or in vitro nucleic acid modification reaction mixture) without requiring the user to take excessive sample processing steps.

The method for processing large volume samples include introducing the biological sample into the sample cartridge. In some instances, the sample can be mixed with reagents prior to introducing it into the cartridge, to disrupt particulates present within the sample. However, the sample introduced into the sample cartridge may also be disrupted in the sample cartridge only when the processing is being carried out.

In order to be able to process a large volume, the volume of the sample chamber and/or lysis chamber within the sample cartridge can be in particular at least 300 μL, at least 500 μL, at least 1,000 μL, at least 1,500 μL, at least 2,000 μL, at least 2,500 μL, at least 3,000 μL, at least 3,500 μL, at least 4,000 μL, at least 4,500 μL, at least 5,000 μL, at least 5,500 μL, at least 6,000 μL, at least 6,500 μL, at least 7,000 μL, at least 7,500 μL, at least 8,000 μL, at least 8,500 μL, at least 9,000 μL, at least 9,500 μL, or at least 10,000 μL. Preferably, the sample cartridge can purify and process nucleic acid from a liquid sample up to 10,000 μL in volume, such as from 300 μL to 5,000 μL, from 300 μL to 3,000 μL, from 300 μL to 2,000 μL, or from 300 μL to 1,000 μL in volume.

It is possible in some cases to disrupt a biological sample in the sample cartridge with a lysis buffer, that is, a solution. In other cases, the biological sample is disrupted prior to introducing into the sample cartridge. Often, it is desirable to treat the sample with enzymes such as lysozyme, proteinase K, and/or a leukoreduction agent before mixing the sample with a chemical lysis buffer. Also, it may be desirable to have more than one lysis buffer and more than one wash buffer. Similarly, aspects of the instrument are not shown that may be used to improve the efficiency of extraction and purification of the large sample volume. For example: 1) a prefilter for capturing the particulate matter in the sample prior to or after lysis, but before sending the lysate over the modified nucleic acid binging filter; 2) elements involved in heating the sample during/prior to lysis, 3) elements involved in sonicating or shearing the sample during lysis, 4) elements involved in sending heated or de-humidified air over the nucleic acid binding matrix that improve drying, and similar features are not shown, but can be assumed to be included to improve the overall performance of the instrument. In some embodiments, multiple rounds of drawing the sample in, then directing the ‘filtered’ sample to waste, can be completed until all the sample or the sample container is left empty.

### Methods

Preparation of Solid Support

Preparation of the solid support or the separation materials described herein can be achieved in any suitable manner. In general, the solid support comprises a reactive group such as a silanol, epoxide, aldehyde, ketone, or activated ester group, so the DNA binding ligands disclosed herein can be attached to the solid support via derivatization reactions, non-covalent coating, or a combination thereof. In some embodiments, the compounds comprising the DNA binding ligand can be covalently attached to the solid support via cycloaddition, nucleophilic or electrophilic substitution, or any other mechanisms well known in the art. FIG. 8, for example, shows an alkylamine coated solid support reacted with cyanuric chloride, and in turn, the stable, electrophilic surface is used to couple amine modified DNA binding ligands. The cyanuric chloride, amino propyl modified glass fiber filter (CC-AP-GFF) can be used in other applications for immobilizing various ligands, such as amine modified oligonucleotides for preparing DNA microarrays.

As described herein, the solid support can include glass fibers which comprise silanol groups. Such glass fiber solid support can be reacted with a silanizing group to obtain the separating materials disclosed herein. Accordingly, the silanol groups of the glass fibers can be reacted with compounds represented by the formula Y-(L)_{y}-SiX₃, wherein each X is independently selected from halogen, alkoxy, dialkylamino, trifluoromethanesulfonate, or a straight, branched, or cyclic alkyl; L is an optional linker such as an alkylene, heteroalkylene linker group, cyanuric chloride, an alkylamine, or a combination thereof and which may be optionally substituted; and Y is a DNA binding ligand, as described herein. The reaction of glass fibers with the compounds described herein provides in glass fibers surface DNA binding groups.

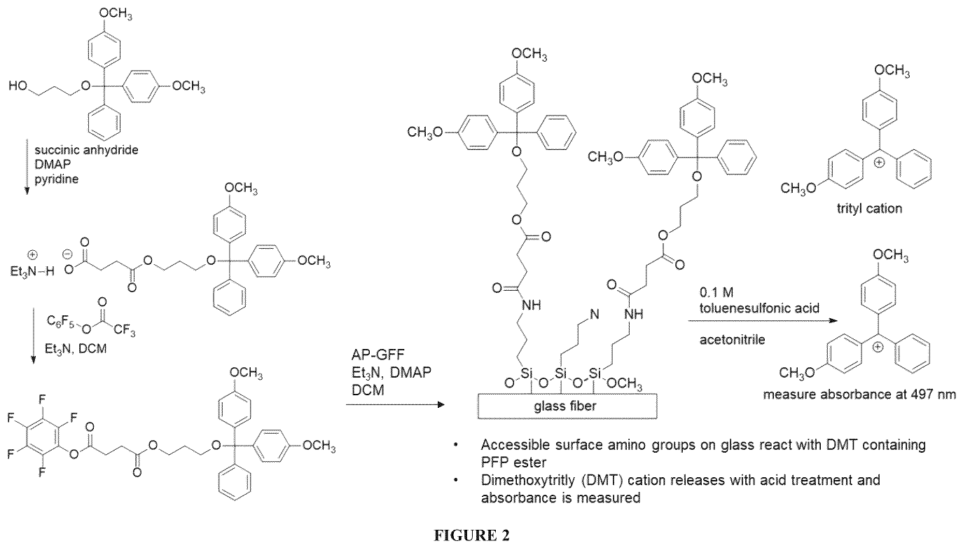

The density of surface DNA binding ligands can be determined using any suitable method, such as the DMT assay provided herein. In the DMT assay, exposed amino or amido groups on the surface of the solid support (such as GFF) react with a pentafluorophenyl (PFP) ester containing a dimethoxytrityl (DMT) reporter group, as described in FIG. 2. Briefly, the method can include treating the modified solid support with DMT containing PFP ester. After washing away excess PFP ester, sterically accessible amino- or amido groups on the solid support surface are left capped with DMT groups. Treatment of the solid support with acid can release the orange trityl cation. Absorbance at 497 nm can be measured with a spectrophotometer. The known DMT extinction coefficient (ε₄₉₇=76,000 M⁻¹ cm⁻¹) and Beers Law can be used to calculate DMT concentration. Amine density is reported in nmoles/cm².

Isolation of Nucleic Acid

Also provided herein are methods for isolation and purification of a nucleic acid from a nucleic-acid containing sample using the solid support disclosed herein. The nucleic-acid containing sample can be selected from blood, plasma, serum, semen, a vaginal swab, a vaginal mucus sample, a vaginal tissue sample, a vaginal cell sample, spinal fluid, tissue, tear, urine, stool, saliva, smear preparation, bacterial culture, mammalian cell culture, viral culture, human cell, bacteria, extracellular fluid, PCR reaction mixture, paraffin-embedded tissue sample, cell lysate, or in vitro nucleic acid modification reaction mixture. In specific embodiments, the nucleic-acid containing sample (biological sample) can be a fixed paraffin-embedded samples (e.g., from FFPET samples) which can be used to identify the presence and/or the expression level of a gene, and/or the mutational status of a gene. In some embodiments, the nucleic-acid containing sample can be a liquid biopsy sample for detection of cancer such as prostate, lung, breast, pancreas, colon, esophagus, ovary, bile duct, stomach, and liver cancers. In some embodiments, the nucleic-acid containing sample can be a respiratory sample for detection of an infectious disease. The nucleic acid-containing sample may comprise human, bacterial, fungal, animal, or plant material. In other embodiments, the nucleic acid-containing sample can be obtained from a nucleic acid modification reaction or a nucleic acid synthesis reaction.

Nucleic acid encompasses any synthetic or naturally occurring nucleic acid, such as DNA or RNA, in any possible configuration, i.e., in the form of double-stranded nucleic acid, single-stranded nucleic acid, aptamer, or any combination thereof. The nucleic acid can be DNA, including dsDNA, ssDNA, and their hybrids. The nucleic acid can also be RNA, such as an mRNA, a non-coding RNA, total RNA, and the like. The nucleic acid can be a synthetic nucleic acid. In some embodiments, the nucleic acid is isolated using the methods described herein are well suited for use in diagnostic methods, prognostic methods, methods of monitoring treatments (e.g., cancer treatment), and the like. Accordingly, the target nucleic acid can comprise genomic DNA, total RNA, short-DNA, small DNA, tumor-derived nucleic acid (including circulating tumor DNA), methylated DNA, microbial nucleic acid, bacterial nucleic acid, viral nucleic acid, cell free nucleic acid, or combinations thereof. In some embodiments, the nucleic acids isolated using the methods described herein are utilized to detect the presence, and/or copy number, and/or expression level, and/or mutational status of one or more cancer markers.

The method for isolation of a nucleic acid from a nucleic-acid containing sample can comprise (a) causing the nucleic acid to contact a solid support comprising a compound having a DNA binding ligand as disclosed herein and (b) eluting the nucleic acid from the modified solid support. As described herein, the nucleic acid can be present as part of a biological sample. In some embodiments, the biological sample is contacted with a lysis solution prior to contacting with the solid support, thereby lysing the cells contained in the biological sample and releasing the nucleic acids into solution. The lysis solution may comprise a chaotropic agent, such as guanidinium thiocyanate, guanidinium hydrochloride, alkali perchlorate, alkali iodide, urea, formamide, and combinations thereof. In some embodiments, the lysis solution may comprise a salt, such as a sodium chloride or calcium chloride salt. In some examples, the lysis buffer comprises one or more of a chaotropic agent, a salt, a buffering agent, a surfactant, a defoaming agent, or a combination thereof. The sample can be lysed by contacting the sample with a lysis buffer prior to contacting the sample with the solid support and subsequent precipitation of nucleic acids. In some instances, the lysis solution comprises one or more proteases. Suitable proteases include, but are not limited to serine proteases, threonine proteases, cysteine proteases, aspartate proteases, metalloproteases, glutamic acid proteases, metalloproteases, and combinations thereof. Illustrative suitable proteases include, but are not limited to proteinase k (a broad-spectrum serine protease), subtilysin trypsin, chymotrypsin, pepsin, papain, and the like. Using the teaching and examples provided herein, other proteases will be available to one of skill in the art.

In some embodiments, the methods disclosed herein do not require the use of a chaotropic reagent or high salt concentration for lysing the nucleic acid containing sample. In some embodiments, the methods disclosed herein can require lower concentrations of a chaotropic reagent or salt for lysing the nucleic acid containing sample, compared to conventional lysis assays. For example, the chaotropic agent can be used in concentrations of less than 4.5 M, less than 2 M, or less than 1 M. In some embodiments, the methods disclosed herein do not require the use of a lysis buffer.

The method of isolating the nucleic acid can further comprise filtering, centrifuging, precipitating, and/or washing the nucleic acid to concentrate the nucleic acid, prior to elution. Conventionally, after nucleic acid lysis, the lysate is filtered on a solid support in the presence of a binding agent (such as PEG) to bind the nucleic acid to the solid support. The binding agent can comprise one or more of an alcohol (e.g., methanol, ethanol, propanol, isopropanol), an alkane diol or alkane triol having 2 to 6 carbon atoms, a monocarboxylic acid ester or dicarboxylic acid diester having 2 to 6 carbon atoms in the acidic component and 1 to 4 carbon atoms in the alcoholic component; a (poly)ethylene glycol and ether derivatives and ester derivatives thereof, and a poly(4-styrene sulfonic acid-co-maleic acid). For example, the binding agent can include one or more of 1,2-butanediol, 1,2-propanediol, 1,3-butanediol, 1-methoxy-2-propanol acetate, 3-methyl-1,3,5-pentanetriol, DBE-2, DBE-3, DBE-4, DBE-5, DBE-6, diethylene glycol monoethyl ether (DGME), triethylene glycol monoethyl ether (TGME), diethylene glycol monoethyl ether acetate (DGMEA), ethyl lactate, ethylene glycol, poly(2-ethyl-2-oxazoline), poly(4-styrene sulfonic acid-co-maleic acid) sodium salt solution, tetraethylene glycol (TEG), tetraglycol, tetrahydrofurfuryl polyethylene glycol 200, tri(ethylene glycol) divinyl ether, anhydrous triethylene glycol, and triethylene glycol monoethyl ether. In some embodiments of the methods disclosed herein, a binding agent is not required, or lower concentrations of binding agents can be used compared to conventional assays. For example, the binding agent such as PEG can be used in concentrations of less than 40% v/v, less than 30% v/v, less than 20% v/v, or less than 10% v/v, of the filtering agent and/or the washing agent. The solid support described herein are coated with a DNA affinity ligand (that is, the DNA binding ligand). Accordingly, the solid support disclosed herein allow selective capture of nucleic acids (RNA and DNA) from biological matrices. Indeed, the modified solid support (such as the modified glass fibers) can capture free circulating nucleic acid as well as nucleic acid from cells without the use of a lysis buffer, salt, or binding agent or with very low concentrations of the same. It is important to point out that the invention described herein encompasses capture of complex genomic DNA or RNA from various organisms in biological samples. Modified glass microscope slides, for example, are commonly used to immobilize DNA or RNA for microarray imaging. In general, these flat, modified surfaces have very low surface area and are not suitable for isolating DNA or RNA from large volumes of complex samples.

The bonded nucleic acid can be optionally washed on the solid support for example, to remove components of the lysis buffer or unwanted components from the biological sample. The concentrated (bonded) nucleic acid can be washed in a buffer compatible with PCR reactions.

The nucleic acid is subsequently eluted from the solid support with an elution buffer. Elution of the nucleic acids off the solid support can be achieved by increasing the pH of the eluent mobile phase or eluting agent, stepwise or in a gradient manner. In some embodiments, the bonded nucleic acid can be eluted from the solid support by contacting with an alkali solution. The alkali solution can comprise ammonia or an alkali metal hydroxide, ammonium hydroxide, NaOH, or KOH in a concentration sufficient for disrupting the binding of the nucleic acid with the compound on the solid support. In some embodiments, the eluting agent has a basic pH. In some embodiments, the eluting agent has a pH of greater than about 9, greater than about 10, greater than about 11, about 9 to about 12, about 9.5 to about 12, about 10 to about 12, or about 9 to about 11. Preferably, the pH of the eluting agent is above 10. Exemplary eluting agents comprise 1% or greater ammonia, 15 mM or greater KOH (e.g., 25 mM KOH, 35 mM KOH, 40 mM KOH, or 50 mM KOH), or 15 mM or greater NaOH (e.g., 25 mM NaOH, 35 mM NaOH, 40 mM NaOH, or 50 mM NaOH). As described herein, the use of high pH to elute nucleic acid such as DNA is unique especially to the cartridges described herein and provides improved speed and performance of the disclosed methods. Speed is provided by the rapid neutralization of acidic ammonium ions by the high concentration of hydroxide ions. A further advantage of the high pH is the denaturing effect of KOH on captured DNA or RNA. Double stranded structures and other secondary structures are disrupted, but can re-nature when neutralized for example, with Tris HCl. The cartridges described herein allows for rapid neutralization of eluted DNA or RNA in KOH/NaOH. A separate Tris reagent (such as in the form of a bead) can be provided to react with the KOH/NaOH eluent instantly to produce a final pH of about 8.5 for downstream PCR or other nucleic acid assays. In some embodiments, the eluting agent has a pH of less than about 9, less than about 8.5, or less than about 8.

In some embodiments, the eluting agent comprises a polyanion. The polyanion is generally a polymer comprising a plurality of anionic groups. In some embodiments, the anionic groups are phosphate, phosphonate, sulfate, or sulfonate groups, or combinations thereof. In some embodiments, the polyanion is a polymer negatively charged at pH above about 7. Both synthetic polyanions and naturally occurring polyanions can be used in the methods disclosed herein. In some embodiments, the polyanion is carrageenan. In other embodiments, the polyanion is a carrier nucleic acid. A carrier nucleic acid, as used herein, is a nucleic acid which does not interfere with the subsequent detection of the concentrated nucleic acid, for example, by PCR. Exemplary carrier nucleic acids include poly rA, poly dA, herring sperm DNA, salmon sperm DNA, and others well known to persons of skilled in the art. In some embodiments, the eluting agent comprises carrageenan and an alkali metal hydroxide, for example, NaOH or KOH.

Overall, strands of nucleic acid including DNA and RNA are readily captured on the solid support surfaces and washed free of impurities at pH 5 or greater. For alkylamine modified glass fiber filters, for example, nucleic acid can be eluted efficiently with high pH buffers (8.5-12.5) or with at least 50 mM KOH as evidenced by PCR assay described herein.

For solid supports modified with a Diels-Alder adducts, the nucleic acid can be optionally released by photochemically or thermally cleaving the adducts. In some instances, the method of isolating nucleic acid includes eluting the nucleic acid from the solid support comprising heating the concentrated (bonded) nucleic acid to a temperature of 100° C. or less, 95° C. or less, 85° C. or less, 75° C. or less, 65° C. or less, 55° C. or less; sonicating the nucleic acid; photochemically cleaving the compound; or a combination thereof, in the presence of an eluting agent.

The nucleic acids isolated using the methods and solid support described herein are of suitable quality to be amplified to detect and/or to quantify one or more target nucleic acid sequences in the sample. Indeed, the nucleic isolation methods and solid support described herein are applicable to use in basic research aimed at the discovery of gene expression profiles relevant to the diagnosis and prognosis of disease. The methods are also applicable to the diagnosis and/or prognosis of disease, the determination particular treatment regiments, and/or monitoring of treatment effectiveness.

Detection of Nucleic Acid

The methods described herein simplify isolation of nucleic acids from biological samples and efficiently produce isolated nucleic acids well-suited for use in RT-PCR systems. In some embodiments, the nucleic acids isolated from a nucleic acid-containing sample using the methods described herein can be detected by any suitable known nucleic acid detection method. Accordingly, methods for detecting a nucleic acid in a biological sample are disclosed. The detection method can comprise nucleic acid amplification. In some embodiments, after eluting the nucleic acid from the solid support with an eluting agent, the methods for detecting a nucleic acid can include combining the eluate with PCR reagents, which may be present in a cartridge as lyophilized particles. In some embodiments, the PCR uses Taq polymerase with hot start function, such as AptaTaq (Roche, Switzerland). The polymerase chain reaction can be a nested PCR, an isothermal PCR, gradient PCR, qPCR, reverse-transcriptase PCR, real-time PCR, multiplex PCR, nucleic acid sequence-based amplification (NASBA), transcription-mediated amplification (TMA), ligase chain reaction (LCR), rolling circle amplification (RCA), or strand displacement amplification (SDA).

In certain embodiments, the method for detecting nucleic acid in a biological sample obtained from a subject can comprise placing the biological sample in a cartridge body as described herein, wherein the cartridge body comprising a plurality of chambers in fluidic communication, a reaction tube configured for amplification of the nucleic acid by thermal cycling, and a filter in the fluidic path between the plurality of chambers and the reaction tube; lysing cells with lysis reagents present within at least one of the plurality of chambers and capturing DNA released therefrom; and amplifying the DNA with primers and probes for detecting the presence of the nucleic acid.

In some embodiments, target nucleic acids, such as coronavirus such as α-coronavirus, β-coronavirus, or SARS-CoV-2, adenovirus, Chlamydia pneumoniae, Influenza A, Influenza B, metapneumovirus, rhinovirus/enterovirus, mycoplasma, Bordetella spp., parainfluenza, and respiratory syncytial virus (RSV), hantavirus, cytomegalovirus, coxsackie virus, herpes simplex virus, echovirus, influenza virus C, Streptococcus pneumoniae, Chlamydia pneumoniae, Moraxella catarrhalis, Haemophilus influenzae, Haemophilus parainfluenzae, a group A streptococcus, Streptococcus pyogenes, Klebsiella pneumoniae, a Pseudomonas species, a Neisseria species, Histoplasnia capsulatum, Coccidioides immitis, Blastomyces dermatitidis, Paracoccidioides brasiliensis, a Candida species, an Aspergillus species, a Mucor species, Cryptococcus neoformans, or Pneumocystis carinii biomarkers and/or optional controls, can be detected. The target nucleic acids can be detected by (a) contacting nucleic acid from the sample with a set of primers and optional probes for detecting the presence of the desired target nucleic acids, (b) subjecting the nucleic acid, primers, and optional probes to amplification conditions; (c) detecting the presence of any amplification product(s), optionally via real-time PCR, melt curve analysis, or a combination thereof, and (d) optionally identifying the presence of the target nucleic acid in the sample, based on detection of the amplification product(s) or lack thereof.

In some embodiments, the amplification method comprises an initial denaturation at about 90° C. to about 100° C. for about 1 to about 10 min, followed by cycling that comprises denaturation at about 90° C. to about 100° C. for about 1 to about 30 seconds, annealing at about 55° C. to about 75° C. for about 1 to about 30 seconds, and extension at about 55° C. to about 75° C. for about 5 to about 60 seconds. In some embodiments, for the first cycle following the initial denaturation, the cycle denaturation step is omitted. The particular time and temperature will depend on the particular nucleic acid sequence being amplified and can readily be determined by a person of ordinary skill in the art.

In some embodiments, the isolation and detection of a nucleic acid is performed in an automated sample handling and/or analysis platform. In some embodiments, commercially available automated analysis platforms are utilized. For example, in some embodiments, the GeneXpert system (Cepheid, Sunnyvale, Calif) is utilized. However, the present invention is not limited to a particular detection method or analysis platform. One of skill in the art recognizes that any number of platforms and methods may be utilized. The GeneXpert system utilizes a self-contained, single use cartridge. Sample extraction, amplification, and detection of a nucleic acid can all be carried out within this self-contained “laboratory in a cartridge.”

Examples of other approaches that can be employed in the methods describe herein include bead-based flow cytometric assay. See Lu J. et al. (2005) Nature 435:834-838, which is incorporated herein by reference for this description. An example of a bead-based flow cytometric assay is the xMAP® technology of Luminex, Inc. See www.luminexcorp.com/technology/index.html. Another approach uses microfluidic devices and single-molecule detection. See U.S. Pat. Nos. 7,402,422 and 7,351,538 to Fuchs et al, U.S. Genomics, Inc., each of which is incorporated herein by reference in its entirety. Yet another approach is simple gel electrophoresis and detection with labeled probes (e.g., probes labeled with a radioactive or chemiluminescent label), such as by northern blotting.

While in some embodiments the extracted nucleic acids are used in amplification reactions, other uses are also contemplated. Thus, for example, the isolated nucleic acids or their amplification product(s) can be used in various sequencing or hybridization protocols including, but not limited to nucleic acid-based microarrays and next generation sequencing.

Readily automated approaches are of great interest. The methods described herein can be carried out in a substantially automated manner using a commercially available nucleic acid amplification system. Exemplary nonlimiting nucleic acid amplification systems that can be used to carry out the methods of the invention include the GENEXPERT® system, a GENEXPERT® Infinity system, and GENEXPERT® Xpress System (Cepheid, Sunnyvale, Calif.). In some embodiments, the amplification system may be available at the same location as the individual to be tested, such as a health care provider's office, a clinic, or a community hospital, so processing is not delayed by transporting the sample to another facility. Assays according to the method described herein can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system. The GENEXPERT® utilizes a self-contained, single-use cartridge. Sample extraction, amplification, and detection may all carried out within this self-contained sample cartridge as described herein.

Prior to carrying out amplification reactions on a sample, one or more sample preparation operations are performed on the sample. Typically, these sample preparation operations will include such manipulations as extraction of intracellular material, e.g., nucleic acids from whole cell samples, viruses and the like to form a crude extract, additional treatments to prepare the sample for subsequent operations, e.g., denaturation of contaminating (e.g., DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced. Preferably, sample preparation is carried out in only one step or no more than two steps. As described herein, the methods simplify isolation of nucleic acids from biological samples and efficiently produce isolated nucleic acids well-suited for use in RT-PCR systems.

The methods for detecting nucleic acid described herein can be effected without transporting the sample from the site where the sample is collected. For example, the method can be carried out at a POC diagnosis location. Locations for the POC diagnosis include a patient care setting, preferably a hospital, an urgent care center, an emergency room, a physician's office, a health clinic, or a home. In some instances, the presence or absence of a nucleic acid can be detected within the biological sample within 75 minutes or within 60 minutes of collecting the sample from the subject.

### EMBODIMENTS

In an embodiment, a method for isolating a nucleic acid from a biological sample, the method comprising: (a) causing the nucleic acid to contact a compound bonded to a glass fiber filter, the compound being derived from a structure represented by the formula:

Y-(L)_{y}-SiX₃
wherein, Y is a DNA binding ligand selected from an alkylamine, a cycloalkylamine, an alkyloxy amine, a polyamine moiety, an arylamine, an intercalating agent, a DNA groove binder, a peptide, an amino acid, a protein, or a combination thereof,
L is a linker selected from an alkyl group, a heteroalkyl group, an alkene group, a heteroalkene group, a polyacrylic acid, a Diels-Alder adduct, or a combination thereof,
each X, independently for each occurrence, is selected from a hydrolyzable group, an alkyl group, a heteroalkyl group, an alkenyl group, or two or three Xs combine to form one or more cyclic groups, or one X combines with Y to form a cyclic azasilane, and
y is 0 or 1; and
(b) eluting the nucleic acid from the glass fiber filter.

In an embodiment, a method for isolation of a nucleic acid from a biological sample, the method comprising: (a) causing the nucleic acid to contact a composition comprising a Diels-Alder adduct, the Diels-Alder adduct including a DNA binding ligand, and (b) concentrating the nucleic acid onto a solid support, wherein the Diels-Alder adduct is optionally bonded to the solid support.

In an embodiment, a method for detecting a nucleic acid in a biological sample, comprising: (a) isolating the nucleic acid from the biological sample using a method as defined in anyone of the embodiments above; (b) eluting the nucleic acid from the solid support with an eluting agent; and (c) detecting the nucleic acid.

In an embodiment, a separating material for nucleic acid isolation comprising: a glass fiber solid support and a compound bonded to a glass fiber solid support, the compound being derived from a structure represented by the formula:

Y-(L)_{y}-SiX₃
wherein, Y is a DNA binding ligand selected from an alkylamine, a cycloalkylamine, an alkyloxy amine, a polyamine moiety, an arylamine, an intercalating agent, a DNA groove binder, a peptide, an amino acid, a protein, or a combination thereof,
L is a linker selected from an alkyl group, a heteroalkyl group, an alkene group, a heteroalkene group, a polyacrylic acid, a Diels-Alder adduct, or a combination thereof,
each X, independently for each occurrence, is selected from a hydrolyzable group, an alkyl group, a heteroalkyl group, an alkenyl group, or two or three Xs combine to form one or more cyclic groups, and
y is 0 or 1.

In an embodiment, a separating material for nucleic acid isolation comprising: a glass fiber solid support comprising a Diels-Alder adduct having a DNA binding ligand, cyanuric chloride, or a combination thereof, wherein the adduct or cyanuric chloride is chemically bonded to a glass fiber solid support, optionally via a linker.

In an embodiment, a sample cartridge for isolation and detection of nucleic acid from a biological sample, comprising: a cartridge body having a plurality of chambers defined therein, wherein the plurality of chambers are in in fluidic communication through a fluidic path of the cartridge, and wherein at least one chamber is configured to receive the biological sample, a reaction vessel configured for amplification of the nucleic acid by thermal cycling, and a filter disposed in the fluidic path between the plurality of chambers and the reaction vessel, wherein the filter comprises a separating material according to any one of the embodiments herein, wherein the plurality of chambers and the reaction vessel independently comprise reagents for releasing nucleic acid from the biological sample, and primers and probes for detection of the nucleic acid.

In an embodiment, a sample cartridge for isolation and detection of nucleic acid from a biological sample, comprising, comprising: a cartridge body having a plurality of chambers therein, wherein the plurality of chambers include: a sample chamber having at least a fluid outlet in fluid communication with another chamber of the plurality; and a lysis chamber in fluidic communication with the sample chamber, the lysis chamber comprising reagents for releasing nucleic acid, optionally wherein the sample chamber and lysis chamber are the same; a reaction vessel fluidically coupled to the plurality of chambers of the cartridge body and configured for amplification of nucleic acid and ii) detection of a plurality of amplification products; a filter disposed in the fluidic path between the lysis chamber and the reaction vessel, wherein the filter comprises a solid support modified with a DNA binding ligand selected from an alkylamine, a cycloalkylamine, an alkyloxy amine, a polyamine moiety, an arylamine, an intercalating agent, a DNA groove binder, a peptide, an amino acid, a protein, or a combination thereof, and a plurality of primers and/or probes disposed in one or more chambers of the plurality of chambers or reaction vessel for detection of the nucleic acid.

In an embodiment, a method for detecting nucleic acid in a biological sample obtained from a subject, the method comprising: placing the biological sample in a sample cartridge according to any one of the embodiments herein; lysing cells optionally with one or more lysis reagents present within at least one of the plurality of chambers and capturing nucleic acid released therefrom; amplifying the nucleic acid with primers and probes for detecting the presence of the nucleic acid.

In an embodiment, a method for detecting nucleic acid in a biological sample obtained from a subject, the method comprising: a) contacting nucleic acid from the biological sample with a set of primers and optional probes in a sample cartridge according to any one of the embodiments herein; b) subjecting the nucleic acid, primer pairs, and optional probes to amplification conditions; c) detecting the presence of amplification product(s), optionally via real-time PCR, melt curve analysis, or a combination thereof, and d) detecting the presence of the nucleic acid in the biological sample based on detection of the amplification products.

In any one of the embodiments above, wherein the DNA binding ligand comprises an amine group, an intercalating agent, a minor groove binder, a peptide, an amino acid, a protein, or a combination thereof.

In any one of the embodiments above, wherein the DNA binding ligand is selected from an alkylamine, a cycloalkylamine, an alkyloxy amine, an arylamine, a polyamine moiety, or a combination thereof.

In any one of the embodiments above, wherein the DNA binding ligand comprises a plurality of amine groups.

In any one of the embodiments above, wherein the DNA binding ligand comprises at least two, at least three, at least four, at least five, at least six amine groups, or a combination thereof.

In any one of the embodiments above, wherein the DNA binding ligand comprises an alkylamine group, an imidazole group, a bisbenzimide minor groove binder, or a combination thereof.

In any one of the embodiments above, wherein the DNA binding ligand is selected from spermine, methylamine, ethylamine, propylamine, ethylenediamine, diethylene triamine, 1,3-dimethyldipropylenediamine, 3-(2-aminoethyl)aminopropyl, (2-aminoethyl)trimethylammonium hydrochloride, tris(2-aminoethyl)amine, or a combination thereof.

In any one of the embodiments above, wherein the Diels-Alder adduct is derived from an unsaturated cyclic imido group.

In any one of the embodiments above, wherein the compound or the Diels-Alder adduct is derived from a structure represented by the general Formula,

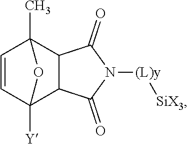

their isomers, salts, tautomers, or combinations thereof, wherein Y′ is the DNA binding ligand, and L, X, and y are as defined in any one of the embodiments herein.

In any one of the embodiments above, wherein the linker, L, is present (or y is 1).

In any one of the embodiments above, wherein the linker is selected from an alkyleneoxy group, an alkylene group, cyanuric chloride, an alkylamine, or a combination thereof.

In any one of the embodiments above, wherein at least two Xs are independently selected from a halogen, an alkoxy, a dialkylamino, a trifluoromethanesulfonate, or combine together with the Si atom to which they are attached to form a silatrane, a cyclic siloxane, a polysilsesquioxane, or a silazane.

In any one of the embodiments above, wherein at least two Xs are independently selected from an alkoxy group (such as ethoxy or methoxy).

In any one of the embodiments above, wherein the compound or the Diels-Alder adduct is derived from one of the following structures:

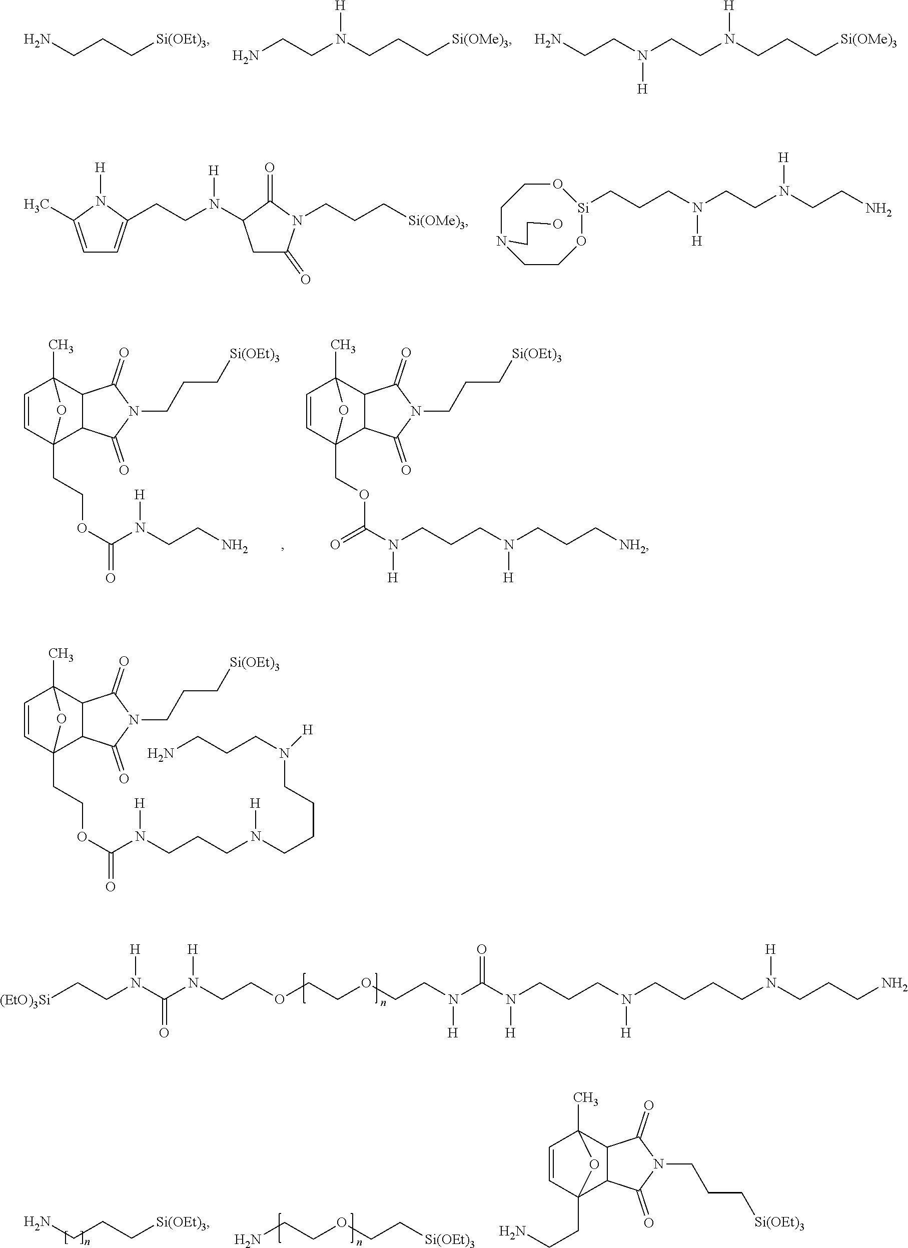

3-aminopropyltrimethoxysilane, an aminoalkylsilatrane, 3-(2-aminoethyl)aminopropyltriethoxysilane, 3-(2-aminoethyl)aminopropyltrimethoxysilane, or a combination thereof, and wherein n is an integer from 0 to 10, from 1 to 10, or from 1 to 5.

In any one embodiments above, wherein at least two Xs are independently selected from an alkoxy group (such as ethoxy or methoxy).

In any one of the embodiments above, wherein the compound or the Diels-Alder adduct is derived from 3-aminopropyltriethoxysilane, 3-aminopropyltrimethoxysilane, or a combination thereof.

In any one of the embodiments above, wherein the solid support comprises a material selected from silica, glass, ethylenic backbone polymer, mica, polycarbonate, zeolite, titanium dioxide, magnetic bead, glass bead, cellulose filter, polycarbonate filter, polytetrafluoroethylene filter, polyvinylpyrrolidone filter, polyethersulfone filter, glass fiber filter or a combination thereof.

In any one of the embodiments above, wherein the solid support is a glass fiber filter.

In any one of the embodiments above, wherein the compound or the Diels-Alder adduct is bonded to the solid support via a siloxane bridge, a carboxylate bridge, as ester bridge, an ether bridge, or a combination thereof.

In any one of the embodiments above, wherein the glass fiber filter has a pore size selected to accommodate correspondingly sized beads to facilitate mechanical lysis.

In any one of the embodiments above, wherein the glass fiber filter has an effective pore size from 0.2 μm to 3 μm, from 0.2 μm to 2 μm, preferably from 0.5 μm to 1.0 μm, or from 0.6 μm to 0.8 μm.

In any one of the embodiments above, wherein the glass fiber filter has a basis weight from 35 g/m² to 100 g/m², preferably from 50 g/m² to 85 g/m², or from 70 g/m² to 80 g/m².

In any one of the embodiments above, wherein the glass fiber filter has a fiber diameter from 1 μm to 100 μm, preferably from 1 μm to 50 μm, or from 1 μm to 25 μm.

In any one of the embodiments above, wherein the glass fiber filter has a thickness from 250 μm to 2,000 μm, from 300 μm to 1,500 μm, from 300 μm to 1,000 μm, from 300 μm to 750 μm, or from 350 μm to 500 μm.

In any one of the embodiments above, wherein the beads are selected from glass beads, silica beads, or a combination thereof.

In any one of the embodiments above, wherein the biological sample is blood, plasma, serum, semen, spinal fluid, tissue, tear, urine, stool, saliva, smear preparation, respiratory sample, nasopharyngeal sample, vaginal swab, vaginal mucus sample, vaginal tissue sample, vaginal cell sample, bacterial culture, mammalian cell culture, viral culture, human cell, bacteria, extracellular fluid, pancreatic fluid, cell lysate, PCR reaction mixture, or in vitro nucleic acid modification reaction mixture.

In any one of the embodiments above, wherein the biological sample is blood, plasma, respiratory sample, or vaginal swab.

In any one of the embodiments above, wherein the biological sample comprises nucleic acid selected from genomic DNA, total RNA, short-DNA, small DNA, tumor-derived nucleic acid, methylated DNA, microbial nucleic acid, bacterial nucleic acid, viral nucleic acid, cell free nucleic acid, or combinations thereof.

In any one of the embodiments above, wherein the biological sample comprises cell free nucleic acid.

In any one of the embodiments above, wherein the biological sample is contacted with a buffer prior to or simultaneously with step a) causing the nucleic acid to contact a composition or a compound bonded to a solid support.

In any one of the embodiments above, wherein the buffer is a lysis buffer comprising one or more of a chaotropic agent, a salt, a buffering agent, a surfactant, a defoaming agent, a binding agent, or a combination thereof.

In any one of the embodiments above, wherein the lysis buffer comprises a chaotropic agent selected from guanidinium thiocyanate, guanidinium hydrochloride, alkali perchlorate, alkali iodide, urea, formamide, or combinations thereof.

In any one of the embodiments above, wherein the chaotropic agent is used at a concentration of less than 4.5 M, less than 2 M, or less than 1 M of the lysis buffer.

In any one of the embodiments above, wherein the method does not utilize a chaotropic agent or a lysis buffer.

In any one of the embodiments above, wherein the buffer comprises saline (inorganic salts such as CaCl₂), MgSO₄, KCl, NaHCO₃, NaCl, etc.), phosphate buffer, Tris buffer, 2-amino-2-hydroxymethyl-1,3-propanediol, HEPES, PBS, citrate buffer, TES, MOPS, PIPES, Cacodylate, SSC, MES, saccharide or disaccharide, or combinations thereof.

In any one of the embodiments above, wherein the nucleic acid is contacted with a binding agent, a filtering reagent, a washing reagent, or a combination thereof, simultaneously with concentrating or prior to eluting the nucleic acid.

In any one of the embodiments above, wherein the filtering reagent and/or the washing reagent comprises the binding agent.

In any one of the embodiments above, wherein the binding agent comprises a polyalkylene oxide (e.g., PEG 200) or a salt.

In any one of the embodiments above, wherein the binding agent is used at a concentration of less than 40% v/v, less than 30% v/v, less than 20% v/v, or less than 10% v/v, of the filtering agent and/or the washing agent.

In any one of the embodiments above, wherein the method does not utilize the binding agent, or the filtering reagent and/or the washing agent does not include a binding agent.

In any one of the embodiments above, wherein the method comprises eluting the nucleic acid with an eluting agent.

In any one of the embodiments above, wherein eluting comprises heating the nucleic acid to a temperature of 100° C. or less, 95° C. or less, 85° C. or less, 75° C. or less, 65° C. or less, 55° C. or less; sonicating the nucleic acid; photochemically cleaving the compound/composition; or a combination thereof, in the presence of an eluting agent.

In any one of the embodiments above, wherein the eluting agent has a pH greater than about 9, greater than about 10, or greater than about 11.

In any one of the embodiments above, wherein the eluting agent has a pH greater than about 10.

In any one of the embodiments above, wherein the eluting agent has a pH of about 10 to about 13.

In any one of the embodiments above, wherein the eluting agent is neutralized with a buffer.

In any one of the embodiments above, wherein the eluting agent is neutralized with an acidic buffer.

In any one of the embodiments above, wherein the eluting agent is neutralized with Tris HCl.

In any one of the embodiments above, wherein the eluting agent has a pH less than about 9, less than about 8.5, or less than about 8.

In any one of the embodiments above, wherein the eluting agent comprises a polyanion, a polycation, ammonia or an alkali metal hydroxide (e.g., NaOH or KOH).

In any one of the embodiments above, wherein the eluting agent comprises a polyanion.

In any one of the embodiments above, wherein the polyanion is a carrageenan, a carrier nucleic acid, or a combination thereof.

In any one of the embodiments above, wherein the method is performed in a cartridge, preferably an automated cartridge.

In any one of the embodiments above, wherein detecting the nucleic acid comprises amplifying the nucleic acid by polymerase chain reaction.

In any one of the embodiments above, wherein the polymerase chain reaction is a nested PCR, an isothermal PCR, qPCR, or RT-PCR.

In any one of the embodiments above, wherein the glass fiber solid support further comprises a polymeric binder.

In any one of the embodiments above, wherein the sample chamber and the lysis chamber are the same.

In any one of the embodiments above, wherein the reaction vessel comprises one or more reaction chambers for detection of the plurality of amplification products.

In any one of the embodiments above, wherein each reaction chamber is configured to detect a single amplification product.

In any one of the embodiments above, wherein each reaction chamber is configured to detect a plurality of amplification products.

In any one of the embodiments above, wherein the cartridge is configured to detect simultaneously a plurality of amplification products present in solution in a single reaction chamber.

In any one of the embodiments above, wherein the cartridge is a Clinical Laboratory Improvement Amendments (CLIA)-compliant cartridge.

In any one of the embodiments above, wherein the cartridge is configured to carry our isothermal amplification.

In any one of the embodiments above, wherein the cartridge is configured to carry out non-isothermal, optionally by thermal cycling, gradient (temperature differential), or temperature oscillation.

In any one of the embodiments above, wherein the sample cartridge further comprising: a syringe that is movable to facilitate fluid flow into and from the lysis chamber by fluctuation of pressure.

In any one of the embodiments above, wherein the lysis chamber comprises: a valve body and a valve cap, wherein the valve body interfaces with the valve cap to define the lysis chamber therebetween, and wherein the filter is held within the lysis chamber secured between the valve body and the valve cap.

In any one of the embodiments above, wherein the lysis chamber has a fluid flow path between an inlet in the cap and an outlet in the valve body that is fluidically coupled to a fluid displacement region of the valve body, wherein the fluid displacement region is depressurizable by movement of the syringe to draw fluid into the fluid displacement region and pressurizable by movement of the syringe to expel fluid from the fluid displacement region.

In any one of the embodiments above, wherein the sample cartridge together with the reagents allow for flow rates up to about 100 μL per second, such as from about 10 μL to about 100 μL.

In any one of the embodiments above, wherein the sample cartridge together with the reagents allow for pressure below 100 psi, below 80 psi, or below 60 psi.

In any one of the embodiments above, wherein the sample cartridge allows for sample volumes up to 1000 μL, such as from 300 μL to 1,000 μL.

In any one of the embodiments above, wherein the lysis chamber further comprises an ultrasonic, piezoelectric, magnetostrictive, or electrostatic transducer to facilitate mechanical lysing.

In any one of the embodiments above, wherein the lysis chamber comprises lysis reagents, the lysis reagents selected from a chaotropic agent, a chelating agent, a buffer, and a detergent to facilitate chemical lysing.

In any one of the embodiments above, wherein the chaotropic agent is selected from guanidinium thiocyanate, guanidinium hydrochloride, alkali perchlorate, alkali iodide, urea, formamide, or combinations thereof.

In any one of the embodiments above, wherein the lysis reagents comprise a guanidinium compound, sodium hydroxide, EDTA, a buffer, and a detergent.

In any one of the embodiments above, wherein the cartridge does not comprise a chaotropic agent.

In any one of the embodiments above, wherein the filter is configured to bind the nucleic acid to be analyzed.

In any one of the embodiments above, wherein the cartridge further comprises a binding reagent, wash reagent, eluting reagent, or a combination thereof.

In any one of the embodiments above, wherein the binding reagent comprises a polyalkylene oxide polymer (e.g., PEG 200) or a salt.

In any one of the embodiments above, wherein the binding agent is used at a concentration of less than 40% v/v, less than 30% v/v, less than 20% v/v, or less than 10% v/v, of the filtering agent and/or the washing agent.

In any one of the embodiments above, wherein the cartridge does not comprise a binding reagent or PEG.

In any one of the embodiments above, wherein the cartridge is an automated cartridge.

In any one of the embodiments above, wherein the cartridge is a single-use disposable cartridge.

In any one of the embodiments above, wherein amplification is by a real-time PCR multiplex assay.

In any one of the embodiments above, wherein: a) said contacting nucleic acid from the sample with the set of primers and optional probes in a sample cartridge comprises: placing the biological sample in the cartridge comprising a cartridge body having a plurality of chambers in fluidic communication, a reaction vessel having one or more reaction chambers and configured for amplification of the nucleic acid, a fluidic path between the plurality of chambers and the reaction vessel, and a filter in the fluidic path; and if the biological sample comprises cells, lysing cells in the biological sample with one or more lysis reagents present within at least one of the plurality of chambers; b) said subjecting the nucleic acid, primer pairs, and optional probes to amplification conditions comprises amplifying the nucleic acid with primers and probes present in solution within at least one of the plurality of chambers; and c) said subjecting the nucleic acid, primer pairs, and optional probes to amplification conditions comprises amplifying the nucleic acid with primers and probes present in solution within at least one of the plurality of chambers.

In any one of the embodiments above, wherein the biological sample is blood, plasma, serum, semen, spinal fluid, tissue, tear, urine, stool, saliva, smear preparation, respiratory sample, nasopharyngeal sample, vaginal swab, vaginal mucus sample, vaginal tissue sample, vaginal cell sample, bacterial culture, mammalian cell culture, viral culture, human cell, bacteria, extracellular fluid, pancreatic fluid, cell lysate, PCR reaction mixture, or in vitro nucleic acid modification reaction mixture.

In any one of the embodiments above, wherein the biological sample is blood, plasma, respiratory sample, or vaginal swab.

In any one of the embodiments above, wherein the method for detecting nucleic acid is for determining the presence or absence of one or more target polynucleotides in the biological sample.

In any one of the embodiments above, wherein the one or more target polynucleotides are selected from genomic DNA, total RNA, short-DNA, small DNA, tumor-derived nucleic acid, methylated DNA, microbial nucleic acid, bacterial nucleic acid, viral nucleic acid, cell free nucleic acid, or combinations thereof.

In any one of the embodiments above, wherein the one or more target polynucleotides comprise cell free nucleic acid.

In any one of the embodiments above, wherein the one or more target polynucleotides is an infectious pathogenic nucleic acid, preferably selected from respiratory pathogen or from coronavirus such as α-coronavirus, β-coronavirus, or SARS-CoV-2, adenovirus, Chlamydia pneumoniae, Influenza A, Influenza B, metapneumovirus, rhinovirus/enterovirus, mycoplasma, Bordetella spp., parainfluenza, and respiratory syncytial virus (RSV), hantavirus, cytomegalovirus, coxsackie virus, herpes simplex virus, echovirus, influenza virus C, Streptococcus pneumoniae, Chlamydia pneumoniae, Moraxella catarrhalis, Haemophilus influenzae, Haemophilus parainfluenzae, a group A streptococcus, Streptococcus pyogenes, Klebsiella pneumoniae, a Pseudomonas species, a Neisseria species, Histoplasnia capsulatum, Coccidioides immitis, Blastomyces dermatitidis, Paracoccidioides brasiliensis, a Candida species, an Aspergillus species, a Mucor species, Cryptococcus neoformans, or Pneumocystis carinii.

In any one of the embodiments above, wherein the biological sample is contacted with a lysis reagent comprising one or more of a chaotropic agent, a salt, a buffering agent, a surfactant, a defoaming agent, a binding agent, a precipitating agent, or a combination thereof.

In any one of the embodiments above, wherein the chaotropic agent is selected from guanidinium thiocyanate, guanidinium hydrochloride, alkali perchlorate, alkali iodide, urea, formamide, or combinations thereof.

In any one of the embodiments above, wherein the chaotropic agent is used at a concentration of less than 2.0 mol/mL of the lysis buffer.

In any one of the embodiments above, wherein the method does not comprise utilizing a chaotropic agent or a lysis buffer.

In any one of the embodiments above, wherein the method further comprises contacting the nucleic acid with a binding agent, a filtering agent, and/or washing to promote binding of nucleic acids to the filter while removing non-target material.

In any one of the embodiments above, wherein the filtering agent and/or the washing agent comprises the binding agent (such as PEG or a salt).

In any one of the embodiments above, wherein the binding agent is used at a concentration of less than 30% v/v of the filtering agent and/or the washing agent.

In any one of the embodiments above, wherein the filtering agent and/or the washing agent does not include a binding agent.

In any one of the embodiments above, wherein the method comprises eluting the nucleic acid with an eluting agent.

In any one of the embodiments above, wherein the nucleic acid is detected within the biological sample within 75 minutes or within 60 minutes of collecting the sample from the subject.

In any one of the embodiments above, wherein amplification is by a real-time PCR multiplex assay.

The following examples are for illustration purposes only, and are not meant to be limiting in any way.

### EXAMPLES

### (a) Example 1: Coated Glass Fiber Filters for DNA Purification

Methods are exemplified for coating glass fiber filters with DNA affinity ligands. Porous glass fiber filters (GFF) are easily fabricated with various DNA affinity coatings to allow selective capture of nucleic acids (RNA and DNA) from biological matrices. Silanization and surface modification methods are exemplified. Strands of DNA and RNA are readily captured on affinity modified glass surfaces and washed free of impurities at pH 5. For alkylamine modified GFF, NA is eluted efficiently with high pH buffers (8.5-12.5). For other affinity capture surfaces (or lower pH elution) cleavable linkers such as esters, photocleavable or thermally cleavable linkers are used. A reliable assay for measuring density of surface alkylamine groups on GFF was developed. Coated GFF having surface density of 30-90 nmoles of alkylamine/cm² have good DNA binding capacity, and release DNA efficiently with 50 mM KOH as evidenced by PCR assay.

Methods to chemically conjugate GFF with DNA binding ligands are shown in FIG. 1. A reliable assay for measuring density of surface alkylamine groups on GFF was developed as shown in FIG. 2. The PFP ester reagent is a novel compound, prepared from succinate, as described herein. A PCR assay was developed to measure DNA extraction performance and is described herein. Briefly, 1%2 inch diameter coated GFFs were assembled into syringe holders and wetted with 0.5 mL volumes of dsDNA or RNA solutions of various concentration at low pH. The amount of DNA that passed through was measured using the GeneXpert following purification of the pass-through solution in a separate cartridge. GFFs were then eluted with 1 mL of high pH buffer and DNA content was measured. The DNA Extraction Score was defined as (Ct of Passthrough−Ct of Elution). A high number (5-12) indicates good extraction performance. A small positive number means weak performance. A negative number means that DNA passed right through (failed to capture). If passthrough and elution Ct are both high, then DNA remained stuck on the GFF (extraction efficiency score=X).

Overall, the modified glass fibers were shown to capture DNA and RNA from solution and subsequently released for PCR tests. GFF modified with amines, polyamines, imidazoles, and BisTris were shown to capture DNA at low pH and release DNA at high pH by a “charge-switch” mechanism. Minor groove binding bis-benzimide (BB) ligands were attached to GFF using cyanuric chloride (CC) activated GFF. While DNA was shown to bind to the BB ligands, the DNA was not efficiently released from the BB coated GFF at high pH BB ligands are fluorescent when bound to dsDNA, and released DNA can be measured at 360 nm or 460 nm to show efficiency of DNA extraction. To solve the release problem of the BB ligands, heat cleavable, aminosilane linkers were attached to GFF, then activated with CC and coated with an amine modified BB ligand.

Materials and Methods.

Two types of borosilicate GFF discs with different nominal pore sizes and thickness were compared. Pall Type A/E had thickness of 0.33 mm and pore size 1 um. Cytiva/Whatman grade GF/F, cat no. 1825-047, Grade GF/F had thickness of 0.43 mm and pore size 0.7 um. 47 mm discs were stacked on a polypropylene cutting board and punched with hammer to yield seven 1%2 inch discs. Cost ˜$0.25/disc. Cytiva discs were also obtained as 0.925 cm laser cut discs (Cepheid, Sunnyvale). Solvents were obtained from Sigma-Aldrich. Anhydrous solvents were handled under argon. Silanes and other organic reagents were obtained from Sigma-Aldrich except for imidazole silane (Boc Chem, China). The PFP ester and BB—NH₂ were prepared using anhydrous technique and chromatographed over 200-400 mesh silica gel using triethylamine in the eluent. Intermediate compounds and final products were analyzed by ¹H NMR (500 MHz) using the indicated deuterated solvents. Despite literature reports, CDCl₃ could not be used with tritylated compounds due to acidity (solution turned orange, hydrolysis peaks visible). UV-vis spectra used a Cary spectrophotometer with a sample cell changer and single beam reading with 1 mL quartz cuvettes, and DMT absorbance was read at 497 nm.

Silanization of ½ inch diameter GFF discs in ethanol (DETA-GFF, Method A). 30 pre-punched ½ inch diameter discs were prepared by stacking several (47 mm diameter) filters for one blow with a steel punch. The required discs were collected in a 50 mL polypropylene screw top centrifuge tube. 30 mL of DETA silanizing reagent (Sigma Aldrich, Cat no. 413348) in absolute ethanol was added at 0.25, 0.5, 1, and 2% by volume). The discs were agitated overnight on a platform rocker and many tubes can be multiplexed. Excess reagent was drained, and discs were washed repeatedly by draining and decanting with fresh volumes of methanol over the course of 3-6 hours (6 times). Discs were drained and excess solvent removed using a vacuum desiccator and high vacuum source (<1 mm Hg) overnight. The discs were stored in the same labeled 50 mL tube used for preparation.

Silanization of 47 mm diameter GFF discs in toluene (AP-GFF, Method B). The APTMS/toluene method used a single 47 mm diameter GFF disc, curled into a 20 mL borosilicate glass scintillation vial. 2 mL of a 0.1 M solution of (3-aminopropyl)trimethoxysilane (APTMS) in toluene (1.8% by weight) was added. The 20 mL vials with foil lined lids were tipped on the side to wet the GFF. he filters became more pliable and translucent when wet with toluene. Several GFF discs can be multiplexed in a rack system. After 2-3 hr, the vials were inverted over a 50 mL tube to drain (nicely sized so they remain suspended over the tube). A 4 mL portion of toluene was added with a Pasteur pipette and 2 mL bulb. The vial was capped and filter shaken briefly to wash off excess reagent. The vial was drained and another 4 mL portion of toluene added/shaken/drained. A final 4 mL volume of toluene was added and the filter soaked 30 min/shaken/drained. The toluene was washed away with 3×4 mL of methanol in the same manner, with 30 min final soak. The discs were drained and removed with tweezers to a clean polypropylene tray. Labeled vial caps were placed over the coated discs during vacuum drying to prevent curling. Either a rotavapor pump (2-5 mm Hg) or high vac pump was used for at least 1 hour. 7 punched 12 inch discs can be obtained from a 47 mm disc as described in Method A and stored in a 20 ml glass vial.

Synthesis of PFP ester (Pentafluorophenyl 3-[bis(4-methoxyphenyl)phenylmethoxy)propyl butanedioate (FIG. 2)). Mono-DMT protected 1,3-propanediol was prepared as described (Seela and Kaiser NAR (1987) 3113) or obtained from Cepheid (SO₃—OH). A solution of 0.59 g (1.56 mmole) of SO₃—OH, 0.44 g (4.4 mmole) of succinic anhydride and 92 mg (0.75 mmole) of 4-dimethylaminopyridine in 12.5 mL of dry pyridine was stirred under argon at room temperature for 4 days. The solution was rotavapped to dryness, then co-evaporated with 2×50 mL of toluene. The residue was dissolved in 2.5% methanol, 2.5% triethylamine (TEA) in dichloromethane (DCM) and chromatographed over 175 mL of silica gel packed in 4×13 cm column packed with same. After 250 mL, product was eluted with 5% methanol, 5% TEA in DCM. Product was collected in ˜200 mL and dried on the rotavap to give 0.83 g (92% yield) of succinylated SO₃—OH (TEA salt) as a colorless syrup after co-evap with 25 mL of toluene. NMR spectrum was not consistent with published spectrum as they did not report the TEA salt (MW=580). The succinate (0.83 g, 1.43 mmole) was converted to PFP ester. 25 mL of DCM and 0.39 mL of TEA was added and septum sealed solution was stirred in ice under argon. A solution of 0.285 mL (0.465 g, 1.66 mmole) of pentafluorophenyl trifluoroacetate (PFP TFA, Aldrich) in 2.5 mL of DCM was added dropwise with syringe. After 1.5 hours, TLC showed no remaining succinate and a single, fast moving DMT+ spot (Rf=0.6, 1:2/Ethyl acetate:Hexane). Rotavap gave 1.37 g of amber syrup, dissolved in 2 mL of ethyl acetate and diluted with 3 mL hexane. The solution was chromatographed over 175 mL of silica gel packed in 4×13 cm column packed with 1:4/E:H. After 200 mL, product was collected in ˜200 mL and dried on the rotavap to give 0.87 g (95% yield) of PFP ester as a colorless syrup. ¹H NMR showed product (CD₃CN) δ 7.45 (d, 2H), 7.34 (m, 7H), 6.88 (d, 4H), 4.23 (t, 2H), 3.78 (s, 3H), 3.14 (t, 2H), 2.95 (t, 2H), 2.66 (t, 2H), 1.90 (m, 2H). 25 mole % ethyl acetate was present. The PFP ester was dissolved in 12.5 mL of dry acetonitrile and the solution was divided into 2 equal portions. One portion of PFP ester was dried in tared flask for −20° C. storage under argon (0.41 g). Another portion was dissolved in dry DMF (6.36 mL) to give a 0.1 M solution, then 1 mL aliquots pipetted to 1.5 mL Eppendorf tubes for cold, dry storage.

DMT assay for measurement of alkylamine density on GFF. The desired number of GFF discs to be tested is determined and required volume of Solution B was prepared (0.3 mL/disc). Solution B includes 1 mL DMF, 0.4 mL TEA, and 0.2 g DMAP. Each test ½ inch diameter test disc was placed in the bottom of a labeled 16 mL vial (Chemglass, CG-4900-03, 21×70 mm, 18-400 thread). To each disc was added 0.300 mL of Solution B and 0.100 mL of PFP ester. The vial was swirled briefly to dislodge any air bubbles, then allowed to stand at least 1 hr. Using a Pasteur pipette and 2 mL bulb, excess reagent was removed from each vial. Then each disc was washed with 3×2 mL of DMF, 3×2 mL of methanol, and 3×2 mL of diethyl ether. As usual, the third wash was allowed to soak at least 30 min before removing. After final ether wash, the vials were vacuum dried for at least 30 min. The dried discs are stable and can be analyzed for DMT content by adding 1.00 mL of 0.1 M p-toluenesulfonic acid in acetonitrile. The orange trityl color is visible immediately, but discs are soaked 30 min before measuring absorbance. If orange color was intense, 0.100 mL was diluted 1:10 dilution with pTos/ACN. A Cary UV-vis spectrophotometer equipped with a 6 cell changer was used in single beam mode with 1 mL cuvettes and pTos/ACN blank baseline subtracted. Absorbance at 497 nm was recorded and DMT concentration for each disc was calculated by dividing by 0.076. Results are given in nmoles/disc. Dividing by 1.27 cm²/disc gives amine density in nmoles/cm².

Syringe filter DNA extraction and PCR assay. Modified glass fiber filter discs were placed within Cytiva Whatman Syringe Filter Holders (cat no. 420100). A 1 mL solution of universal transport medium containing 500 copies per mL genomic DNA from Streptococcus pyrogenes (bacterial strain Bruno ATCC 19615) was passed through the modified filter discs. The passthrough solution was transferred into a Cepheid RCC Cartridge (having unmodified glass fiber filter with acrylic binder, 1 micron pore size, 50 mils thickness) for re-purification, polymerase and oligonucleotide introduction, thermal cycling and fluorescence detection. The same modified filter discs each additionally had 1 mL of 50 mM KOH solution passed through them to elute any residual DNA from the filter discs. This eluate was processed identically but within a separate Cepheid RCC Cartridge. The passthrough and eluate conditions were analyzed relative to each other according to their Cycle Count (Ct) and End Point Fluorescence (EPF) Values. These values were analyzed relative to a control condition of having placed 1 mL solution of universal transport medium containing 500 copies per mL genomic DNA directly into one of the Cepheid RCC Cartridges.

Synthesis of CL-53

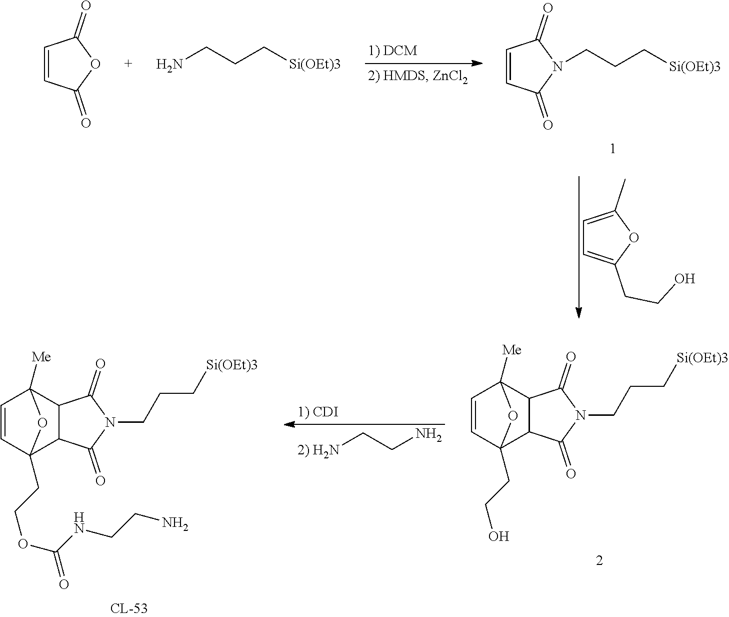

Step 1. (3-Aminopropyl)triethoxysilane (10.76 g, 48.61 mmol) was weighed into a dry 500 mL RB flask and placed under argon. 100 mL of anhydrous DCM was added and the mixture cooled to 0° C. Maleic anhydride (4.75 g, 48.4 mmol) was added and the reaction warmed to RT and stirred at RT for 3.5 h. The mixture was diluted with 250 mL of toluene and the DCM removed using a rotary evaporator. ZnCl₂ (7.29 g, 53.5 mmol) and hexamethyldisilazane (11.10 mL, 53.66 mmol) were added and the mixture heated at 100° C. for 14 h. Solids were filtered off and toluene removed in vacuo, followed by drying under high vacuum overnight. Crude compound 1 (14.22 g, 97%) was obtained and used without further purification. ¹H NMR (CDCl₃, 500 MHz): δ 6.67 (s, 2H), 3.80 (q, J=7.0 Hz, 6H), 3.50 (t, J=7.4 Hz, 2H), 1.68 (m, 2H), 1.21 (t, J=7.0 Hz, 9H), 0.58 (m, 2H).

Step 2. Compound 1 (4.78 g, 15.9 mmol) and 2-(5-methylfuran-2-yl)ethanol (2.00 g, 15.9 mmol) were dissolved in 15 mL of reagent grade ethanol and stirred at RT for 24 h. The ethanol was then removed in vacuo with a water bath set to 30° C. (do not heat compound). The mixture was chromatographed on silica gel (1.5″×6″) using EtOH/hexane (5 to 10% ethanol) and both the endo (3.25 g, 48%) and exo (1.81 g, 27%) isomers isolated cleanly. Isomer assignment based on literature, De Bo, G., JACS, 2017, 139, 16768-16771. Endo isomer ¹H NMR (DMSO-d₆, 500 MHz): δ 6.31 (d, J=5.6 Hz, 1H), 6.17 (d, J=5.6 Hz, 1H), 4.54 (s, 1H), 3.71 (q, J=7.0 Hz, 6H), 3.59 (t, J=7.0 Hz, 2H), 3.41 (d, J=7.5 Hz, 1H), 3.24 (d, J=7.4 Hz, 1H), 3.16 (t, J=7.4 Hz, 2H), 2.21 (m, 1H), 2.11 (m, 1H), 1.63 (s, 3H), 1.38 (m, 2H), 1.11 (t, J=7.0 Hz, 9H), 0.45 (m, 2H). Exo isomer ¹H NMR (DMSO-d₆, 500 MHz): δ 6.50 (d, J=5.4 Hz, 1H), 6.34 (d, J=5.4 Hz, 1H), 4.53 (s, 1H), 3.67 (q, J=7.1 Hz, 6H), 3.63 (m, 2H), 3.33 (t, J=6.9 Hz, 2H), 2.93 (d, J=6.3 Hz, 1H), 2.85 (d, J=6.3 Hz, 1H), 2.11 (m, 1H), 1.96 (m, 1H), 1.52 (s, 3H), 1.48 (m, 2H), 1.11 (t, J=7.1 Hz, 9H), 0.46 (m, 2H).

Step 3. Under argon, in a dry RB flask, compound 2 (endo isomer, 606 mg, 1.42 mmol) was dissolved in 20 mL of anhydrous DCM. Diisopropylethylamine (500 uL, 2.26 mmol) and carbonyldiimidazole (252 mg, 1.55 mmol) were added and the reaction was stirred a RT for 24 h. Ethylenediamine (190 uL, 2.85 mmol) was added and the reaction was stirred at RT for another 2 h. The mixture was then filtered through filter paper, diluted with DCM, and extracted with water (3 times) and brine. The filtrate was dried over Na₂SO₄ and the solvent removed in vauco to give 1.45 g (88%) of CL-53. ¹H NMR (DMSO-d₆, 500 MHz): δ 7.09 (t, J=5.5 Hz, 1H), 6.34 (d, J=5.4 Hz, 1H), 6.22 (d, J=5.6 Hz, 1H), 4.40 (bs, 2H), 4.09 (t, J=6.8 Hz, 2H), 3.69 (q, J=7.0 Hz, 6H), 3.44 (m, 1H), 3.25 (d, J=7.4 Hz, 1H), 3.16 (t, J=7.4 Hz, 2H), 2.96 (q, J=6.0 Hz, 2H), 2.54 (t, J=6.5 Hz, 2H), 2.36 (m, 1H), 2.21 (m, 1H), 1.64 (s, 3H), 1.38 (m, 2H), 1.10 (t, J=7.0 Hz, 9H), 0.46 (m, 2H). LCMS: found m/z 514.2 [M+H], calc. 513.7.

Synthesis of CL-54

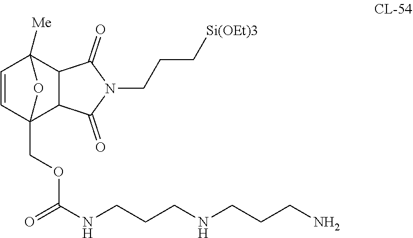

Synthesis of CL-54 followed the same procedure as CL-53, however in Step 2, 2-(5-methylfuran-2-yl)methanol (1 eq) was used in place of 2-(5-methylfuran-2-yl)ethanol, and bis(3-aminopropyl)amine (3 eq) was used in place of ethylenediamine in step 3. LCMS: found m/z 571.3 [M+H], calc. 570.8.

Synthesis of CL-56

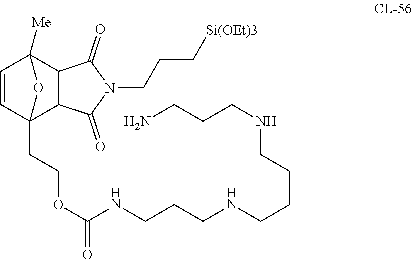

Synthesis of CL-56 followed the same procedure as CL-53, however spermine (1 eq) was used in place of ethylenediamine in Step 3. LCMS: found m/z 656.4 [M+H], calc. 655.9.

Synthesis of succinylated AP-GFF. A 47 mm diameter aminopropyl coated GFF was prepared using Method B. The disc was curled into a 20 mL scintillation vial and treated with a solution of 0.06 g of succinic anhydride and 10 mg DMAP in 2 mL of anhydrous pyridine. After 2 hours, the reagent solution was removed and discs were washed with 3×3 mL of pyridine, 3×3 mL of methanol and 3×3 mL of DCM. Vacuum drying for at least 30 min gave succ-AP-GFF discs.

Synthesis of BisTris-succ-AP-GFF. A 47 mm diameter disc of succinylated aminopropyl glass fiber filter (succ-AP-GFF) was coated with BisTris (MW 209). The disc was inserted into 20 mL scintillation vial as usual and a solution of 100 mg of bis-tris in 1 mL of 50 mM imidazole buffer (pH 6.0) was added. A solution of 100 mg of EDC-HCl (MW 191.7) in 1 mL of the same buffer was prepared and immediately added to the succ-AP-GFF disc. The homogenous solution was allowed to react with the disc overnight, then the filter was washed with 3×4 mL of water and 3×4 mL of methanol as usual. The disc was dried under vacuum and tested for DNA binding and release in the PCR assay.

Tricine-succ-AP-GFF. A 47 mm diameter disc of succinylated aminopropyl glass fiber filter (succ-AP-GFF) was coated with Tricine (MW 179). The procedure was as described above for Bis-Tris, except that 50 mg of Tricine was used. The disc was dried under vacuum and tested for DNA binding and release in the PCR assay.

Cyanuric chloride activated AP-GFF (CC-AP-GFF). A 47 mm diameter disc of aminopropyl coated glass fiber filter (AP-GFF, 59 nmole/cm²) was reacted with 2 mL of a 0.1 M solution of cyanuric chloride (CC) in toluene. 37 mg (0.2 mmoles) of CC was weighed into a 20 mL scintillation vial and 2 mL of toluene was added. The mixture was stirred until dissolved then the disc inserted. The vial was sealed and tipped over to soak the disc at the bottom. After 3 hours, the solution was removed with a Pasteur pipette and washed with 3×4 mL of toluene as usual. The disc was washed with methanol and diethyl ether, then dried under vacuum for storage. ½ inch diameter discs were punched from the 47 mm disc of CC-AP-GFF disc. One ½ inch disc was tested for residual amines (found 3 nmole/cm²). A second 1%2 inch disc was tested for amine reactivity. The disc was treated with a 1 M solution of propylene diamine (PDA) in DMF (28 μL of PDA, 0.4 mL DMF in a 16 mL screw cap vial). After overnight reaction, the disc was washed with DMF, methanol and diethyl ether. The test CC-AP-GFF disc was then measured for amine content as usual to yield (27 nmole/disc). Remaining five CC-AP-GFF discs were treated with bis-benzimide NH₂ (BB—NH₂) as described below. Unused CC-AP-GFF was stored in a refrigerator.

Cyanuric chloride activated CL53-GFF (CC-CL53-GFF). CL53-GFF was prepared using Method B and tested for amine loading (32 nmole/cm²). This material was also tested in the PCR assay, having a DNA extraction score of 12). A separate 47 mm disc of CL53-GFF was treated with CC/toluene for 3 hours as described above. The filter was not evaluated for amine content or reacted with PDA. Instead, the disc was used directly for immobilization of bis-benzimide amine as described below.

Synthesis of Bis-Benzimide hexylamine (BB—NH₂). The synthesis of BB—NH₂ used the procedure of Reed et al. (US 2006/0166223) with the following modifications. Synthesis started with 500 mg of Hoechst 33258 vs. the published 9.5 mg scale. Silica gel purification gave 380 mg of Boc protected aminohexyl-BB (65% yield). 125 mg of BB-NHBoc was deprotected with 10 mL of trifluoroacetic acid and 10 mL of chloroform. After 18 hours the solution was dried on a rotovap to give 340 mg of orange syrup. NMR showed BB—NH₂, bis-TEA salt. This material was dissolved in DMF and used for immobilization reactions, using Solution B to ensure the free base form of BB—NH₂.

Immobilization of Bis-benzimide hexylamine (BB—NH₂) to CC-AP-GFF. A 47 mm diameter disc of CC-AP-GFF was placed in a 20 mL vial. A 0.1 mM solution of BB—NH₂ (0.123 mmol in 1.23 mL DMF) was mixed with 1 mL of Solution B (1 mL DMF, 0.4 mL TEA, 20 mg DMAP) and added to the disc. The disc was soaked overnight, then washed with 5×4 mL portions of DMF, and 5×4 mL of methanol. The disc was dried under vacuum and tested for DNA binding/release in the PCR assay.

Immobilization of BB—NH₂ to CC-CL53-GFF. BB—NH₂ (0.148 g, ˜0.123 mmole) was dissolved in 1 mL of dry DMF and mixed with 1 mL of Solution B. A 47 mm disc of CC-CL53-GFF was inserted into 20 mL scintillation vial and the BB—NH₂ solution was added. The filter was soaked overnight, then washed with 5×4 mL of DMF and 5×4 mL of methanol as usual. The discs were dried under vacuum and stored refrigerated prior to testing for DNA extraction efficiency in the PCR assay.

Results and Discussion

Comparison of silanization methods for amine coated GFF. Trialkoxysilanes can be used to coat glass surfaces using a variety of solution phase and gas phase methods. Described herein are methods that use either protic solvents (Method A, ethanol) or aprotic solvents (Method B, toluene). Method A was used to prepare EDA, DETA, PEG-spermine and imidazole coated GFF. Method A was also used to coat GFF with aminosilanes containing heat cleavable linkers (CL53, CL54, CL55). Method B was found to give ˜2 times higher amine density than Method A. For example, amine density of Pall discs increased from 17 nmole/cm² (Method A) to 41 nmole/cm² (Method B). Whatman discs increased from 39 nmole/cm² (Method A) to 88 nmole/cm² (Method B). Reaction of trimethoxysilane groups in APTMS requires displacement by surface silanol groups to form siloxane bonds on the GFF surface. Without wishing to be bound by theory, it is believed that in ethanol, the solvent can reverse this reaction, whereas in toluene, the displaced ethanol dissolves (low concentration). Method A, however, was more convenient and used easily with pre-punched discs, but overnight washing added time. On the other hand, Method B was used to prepare higher loading AP-GFF discs requiring further reactions to attach DNA binding ligands (2 step Process) and used 0.1 M APTMS (2% wt/volume). Ethylenediamine (EDA) silane coating used Sigma Aldrich, Cat no. 104884 and Method A. Epoxy silane coating of GFF used (3-glycidyloxypropyl)triethoxysilane (Sigma Aldrich, Cat no. 50059) and Method B. Imidazole silane coating used N-(trimethoxysilylpropyl)imidazole (Boc Sci, Cat no. 70851-51-3) and Method A. PEG-spermine silane was prepared from APTES with average PEG linker length of 45 units and immobilization used Method A.

Structures, amine density, and DNA extraction performance of some amine coated GFF are shown Table 1. The methods were also used to attach aminoalkylsilanes with heat cleavable linker structures (CL) to GFF, as described in Table 3.

TABLE 1
Structure, amine density and DNA extraction performance of Alkylamine coated GFF.

| | | Amine | | Pass | DNA |
|---|---|---|---|---|---|
| Silane reagent/ | Structure/ | density | Eluate | through | Extraction |
| GFF type | Method | (nmole/cm²) | Ct | Ct | Score |
| APTMS (2%)/Pall | Aminopropyl/A | 17 | 32.6 | 36.8 | 4 |
| APTMS (2%)/Whatman | Aminopropyl/A | 39 | 33.8 | 43.1 | 9 |
| APTMS (2%)/Pall | Aminopropyl/B | 41 | 33.7 | 36.4 | 3 |
| APTMS (2%)/Whatman | Aminopropyl/B | 88 | 32.4 | 43.8 | 11 |
| APTES (0.5%)/Whatman | Aminopropyl/A | ND | 32.1 | 37.3 | 5 |
| APTES (1.0%)/Whatman | Aminopropyl/A | ND | 31.7 | 45 | 13 |
| APTES (2.0%)/Whatman | Aminopropyl/A | ND | 32.8 | 41.8 | 9 |
| DETA (0.5%)/Pall | diethylenetriamine/A | 25 | 34.4 | 38.8 | 4 |
| DETA (1.0%)/Pall | diethylenetriamine/A | 27 | 34.3 | 37.4 | 3 |
| DETA (2.0%)/Pall | diethylenetriamine/A | 37 | 35 | 40.4 | 5 |
| PEG-spermine/Pall | PEG15 linker/A | 20 | 33.5 | 45 | 12 |
| Imidazole/Whatman | N-propylimidazole/A | ND | 32.7 | 43.4 | 11 |

Except for APTMS, all GFF were silanized using Method A. PEG spermine (avg MW 2476) was immobilized at 8.3 mg/mL. Except for imidazole-GFF, amine density was measured with the DMT assay. DNA was eluted using 50 mM KOH. Imidazole was also eluted with pH 8.5 Tris (not shown). DNA Extraction Score=Ct of Passthrough−Ct of Elution.

Methods A and B both avoided a 30 min high temperature (100° C.) “curing” step that is commonly used to prepare silanized glass surfaces. The high temperature heating step presumably provides more stable surfaces by increasing crosslinking of the multilayer silane coating, but it may be expected that heating decreases amine density on GFF surfaces. Heating may convert surface ammonium groups to the (less stable) free base which can form irreversible N-oxides. Methods described here provide good amine density and stability without this damaging heating step. The resulting “vacuum cured” AP-GFF surface is stable when dry at room temp. AP-GFF filters survive brief treatment with 50 mM KOH (pH 12.5) for elution of nucleic acid, but the DMT assay showed 30 min soaking with 50 mM KOH cut surface amine density in half (data not shown).

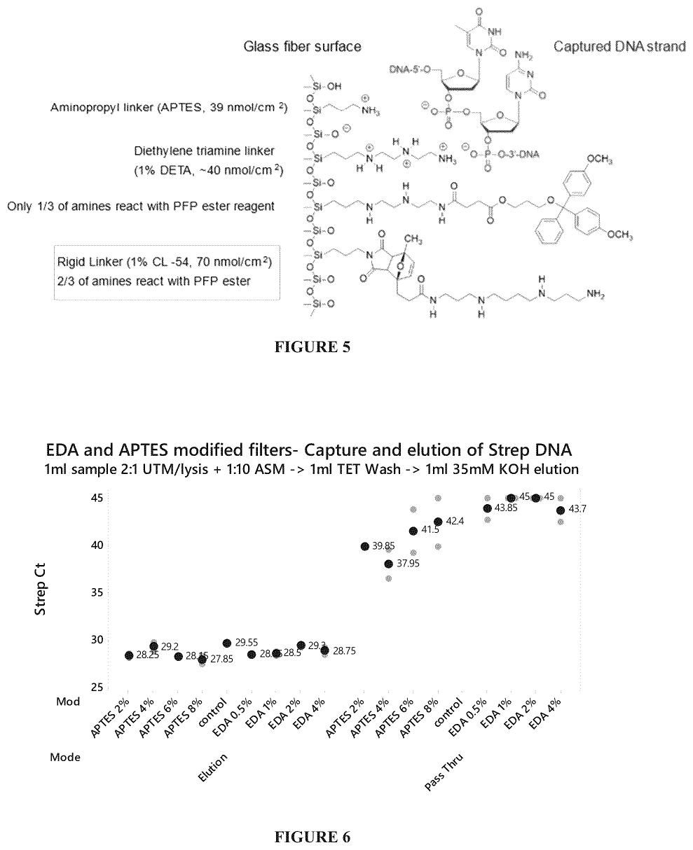

After validating the DMT assay with AP-GFF, amine density of other alkylamine coated GFF was examined. GFFs were prepared using Pall filters and Method A to compare APTMS (mono-alkylamine), DETA (tri-alkylamine) and PEG45-spermine (tri-alkylamine). The coatings had amine density as shown in Table 1. The DETA coating had ˜50% higher amine density (25-37 nmole/cm²) vs. APTMS (17 nmole/cm²), not triple. The pentafluorophenyl (PFP) ester only reacts with sterically accessible amine groups on the GFF surface. It is believed that the polyamines are electrostatically bound to surface silanols on the GFF as shown in FIG. 4. Alternately, the secondary amines in the aliphatic polyamine linker may be too sterically hindered for reaction of the PFP ester. The long PEG linker did not improve accessibility of the attached spermine since DMT assay showed similar amine loading (20 nmole/cm²) to the monoamine APTMS. EDA (di-alkylamine) was evaluated (Whatman GFF) and showed ˜50% higher amine density vs. APTES (FIG. 6).

DMT assay for alkylamine groups on GFF. Exposed amino groups on the surface of GFF react with a pentafluorophenyl (PFP) ester containing a dimethoxytrityl (DMT) reporter group, as described in FIG. 2. Synthesis of the PFP ester from succinate is described herein. The (pure, stable) PFP ester was used instead of p-nitrophenyl (PNP) esters or tetrafluorophenyl (TFP) esters.

The DMT PFP ester assay was developed using ½ inch diameter AP-GFF discs and rapid acylation reaction conditions. Briefly, an AP-GFF disc (layed flat in a 16 mL vial) was treated with a DMT containing 0.4 mL of PFP ester in a DMF solution containing DMAP and triethylamine. AP-GFF showed complete reaction of surface amines within 20 min (FIG. 3).

After washing away excess PFP ester, sterically accessible amino groups on the GFF surface are left capped with DMT groups. Treatment of the disc with 1.00 mL of acid (0.1 M p-toluenesulfonic acid in acetonitrile) releases the orange trityl cation. Absorbance at 497 nm is measured with a spectrophotometer (1:10 dilutions required). The known DMT extinction coefficient (ε₄₉₇=76,000 M⁻¹cm⁻¹) and Beers Law were used to calculate DMT concentration. Amine density is reported in nmoles/cm² (each ½ inch disc is 1.27 cm²). Reacting the PFP ester for longer times did not increase amine density significantly. Details of the DMT assay for GFF amine density assay are given herein.

PFP ester amide bond formation with AP-GFF is concentration dependent. The published method for measuring surface amines in porous supports used equal volumes of 0.1 M PNP ester and Solution B (1 mL DMF, 0.4 mL triethylamine, 40 mg of dimethylaminopyridine). For DMT assay of GFF, the concentration of PFP ester was varied and measured the resulting DMT concentration. To completely wet GFF discs in the 14 mL vial, 0.2 mL of total volume was used. Then 0.2 mL of PFP ester in DMF was added at the concentration in FIG. 3. After 2 hours of reaction, the discs were washed as usual. Two different GFF types were also compared. As expected, the lower surface area Pall AP-AP-GFF gave lower DMT loading than the thicker Whatman GFF. This experiment also showed that DMT loading increases as PFP ester concentration increases. For Pall discs, max DMT loading (36 nmole/cm²) was achieved with 0.05 M (25 mM final conc). For Whatman discs, DMT loading increased from 75 to 88 nmole/cm² with 0.1 M PFP ester (17% increase).

Sterically accessible surface amines must “stick out” from the inner layer, and not be bound to other silanol or alkoxysilane groups (FIG. 4). The free base form of surface propylamines reacts rapidly with properly aligned PFP ester reagent. Triethylamine in “Solution B” acts as a proton scavenger to prevent non-productive ammonium ion formation. DMAP (4-dimethylamino pyridine) further acts as a catalyst for amide bond formation. Anhydrous DMF (dimethylformamide) is used to prevent hydrolysis of the PFP ester reagent. Amide bond formation in this polar, aprotic solvent is further driven to completion by the huge molar excess of PFP ester. For example, a 1.27 cm² AP-GFF Whatman disc was soaked with 0.2 mL of 0.1 M PFP ester (20 μmoles). Density of 88 nmole/cm² (112 nmole/disc) of DMT was measured. That is a DMT molar excess of 20,000/112=180 molar equivalents.

These chemical factors drive the PFP ester to react with all accessible amines on the GFF surface with 50 mM final conc. To routinely measure amine density of amine coated GFF, there is no need to exaustively cap with DMT. To conserve reagent, the assay described herein uses 100 μL of 0.1 M PFP ester in DMF. Then 300 μL of Solution B is added to give 25 mM final concentration of PFP reagent. We use this method routinely to describe amine density of GFF, but expect 17% higher amine loadings could be achieved with 50 mM PFP ester concentration, as shown in FIG. 3.

After thorough washing and drying as described herein, the PFP ester treated AP-GFF discs are stable, and can be stored in the 14 mL reaction vial for future DMT assay. DMT cation color fully develops after 30 min of soaking with 1.00 mL of 0.1 M p-toluene sulfonic acid in acetonitrile (easily dispensed with pipettor). Absorbance at 497 nm of the resulting orange solutions was measured in quartz cuvettes with 0.1 M pTos/ACN blanks. 1:10 dilutions were required for on-scale measurement of the 88 nmole/cm² AP-GFF disc (A₄₉₇=0.817 au).

DMT assay of unmodified GFF (negative control). As shown in FIG. 3, DMT level in the blank Pall disc was negligible (1.5 nmole/cm²). But the Whatman disc showed significant DMT content (8.7 nmole/cm²). The increase with the greater surface area Whatman GFF is likely the reason for the increase in background, but there may be a difference in the surface chemistry of the Pall GFF. It is possible the PFP ester acylates high pKa silanols on the glass surface. These sites likely react with trialkoxyaminosilanes, and so this “background” is not subtracted from the DMT measurements reported in Table 1. When AP-GFF discs are succinylated, background DMT measurements drop to 1.3 nmole/cm². This low background for the Whatman discs shows that the DMT assay can reliably wash away unreacted or hydrolyzed PFP ester.

DMT assay of AP-GFF disc (positive control). AP-GFF discs were made using Method B (49 punched ½ inch discs). Unpunched, vacuum dried 47 mm discs were stored in brown glass jars. Punched discs were stored in 20 mL scintillation vials. Amine density was analyzed over time (Table 2). It is interesting that the first few AP-GFF amine density measurements were higher. It may be variation of the DMT assay, variation of surface area in the discs, or actual variations in the amine density. In any event, AP-GFF is stable for at least 2 months.

TABLE 2
Stability of AP-discs over time (shelf-life)

| Assay | AP-GFF Amine density | GFF Amine density |
|---|---|---|
| date | nmole/cm² | nmole/cm² |
| Day 1 | 63 | 7.9 |
| Day 5 | 66 | 8.7 |
| Day 22 | 59 | 9.0 |
| Day 33 | 59 | 7.2 |
| Day 51 | 59 | 9.0 |
| Day 79 | 69 | 9.3 |

AP-GFF (Whatman) were prepared using Method B. DMT assay was used to calculate amine density using 50 mM PFP ester. AP-GFF discs had mean of 61 (+/−3). Unmodified control GFF control discs had mean of 8.4 (+/−0.8).

The DMT assay showed higher amine density in Whatman GFF coated with the heat cleavable linkers (Table 3). As shown in FIG. 5, CL linkers have a rigid linker structure that helps the amine “stick out” for the surface or prevent the electrostatic surface binding of aliphatic polyamines. In Table 1, Pall GFF with 1% DETA silane (Method A) gave amine density of 27 nmole/cm², compared to 41 nmole/cm² for AP-GFF (Method B). As shown in Table 3, triamine CL-56 silane on Whatman GFF (Method A) gave higher amine density (70 nmole/cm², comparable to 63 nmole/cm² for AP-GFF (Method B).

It is unclear if increased amine density with CL54 is due to denser packing during silanization or improved accessibility of the PFP ester during the DMT assay. In any case, measured density of alkylamine groups relates to steric accessibility during DNA capture. Table 3 shows that amine density of the CL did not change much with increasing silane amounts from 0.25-1%. Despite higher amine loading, DNA extraction performance with CL54-GFF and CL-56 did not improve. These filters bound DNA efficiently (high passthrough Ct) but DNA did not release well with 50 mM KOH (high elution Ct). The DNA extraction score was therefore lower than the APTMS control. CL53-GFF did show good DNA release at high pH. DNA extraction performance of the Cleavable Linker GFFs were further studied with heat cleavage experiments as described below. Structures and synthesis of the CL linkers are described herein.

Aminoalkyl coated GFF discs. The preferred dimensions of GFF material for modification are strips (1.6×11 cm). The ½ inch discs or strips can be coated by soaking free floating (agitated) GFF pieces in ethanolic silanizing solution (Method A). For assembly into extraction cartridges, 9.25 mm diameter discs can be laser cut from GFF. For assembly line, GFF is also available in rolls. 90 mm discs are available and fit into 100 mm Nalgene Petri dishes to yield 30×½ inch diameter discs.

TABLE 3
Structure, amine density and DNA extraction performance with CL silanes on GFF.

| | | | | % DNA eluted | | | DNA |
|---|---|---|---|---|---|---|---|
| Silane reagent/ | | Amine density | Capture | @95° C. TET | | | extraction |
| GFF type | Structure | (nmole/cm²) | (%) | 2 min | 5 min | 10 min | Score* |
| CL53 (0.25%)/Pall | Heat | 39 | 100 | 81 | 66 | 100 | 9 |
| | cleavable, | | | | | | |
| | monoamine | | | | | | |
| CL53 (0.5%)/Pall | | 38 | 100 | 81 | 100 | 100 | 9 |
| CL53 (1%)/Pall | | 33 | 100 | 57 | 81 | 100 | 7 |
| CL54 (0.25%)/Pall | Heat | 77 | 100 | 0 | 0 | 27 | 1 |
| | cleavable, | | | | | | |
| | diamine | | | | | | |
| CL54 (0.5%)/Pall | | 72 | 100 | 0 | 0 | 22 | 1 |
| CL54 (1%)/Pall | | 70 | 100 | 0 | 2 | 19 | 1 |
| CL56 (0.25%)/Pall | Heat | 64 | 97 | 0 | 22 | 23 | 1 |
| | cleavable, | | | | | | |
| | triamine | | | | | | |
| CL56 (0.5%)/Pall | | 70 | 100 | 2 | 2 | 20 | 1 |
| CL56 (1%)/Pall | | 71 | 100 | 0 | 1 | 7 | 0 |

GFF was silanized using Method A. DNA was eluted using pH 8.6 TET (Tris, EDTA, Tween-20). DNA Extraction Score (Ct of Passthrough−Ct of Elution) was calculated for 2 min elution times at 95° C.

Two-step process for attaching DNA binding ligands to GFF. Alkylamine coated GFF described above can be used as a starting material to attach DNA binding ligands for which no silane exists or are incompatible with the silanization process. Nucleophilic amine coated surfaces can react with electrophilic carboxylic acid ligands to form amide bonds. This is exemplified by the DMT PFP reagent. Carboxylic acid ligands can also be activated in situ with carbodiimide coupling agents like EDC. The surface alkylamines are easily converted to succinic acid functionalized GFF (FIG. 1, step 2). The succinates can be activated to form amide bonds with amine containing ligands. Methods for succinylating GFF are described herein. The method successfully attach Bis-Tris with EDC coupling chemistry. Amine containing ligands can also be attached to GFF coated with epoxy silanes. Although epoxy silane was successfully attached to GFF using Method B, a more efficient method for immobilizing DNA binding ligands was discovered. The examples describe cyanuric chloride activated AP-GFF and CL53-GFF. Finally, immobilization of bis-benzimide (BB) minor groove binder is described.

Succinylation of AP-GFF. Conjugation chemistry of functionalized AP-glass filter fibers (GFF) is analogous to the well-known chemistry of modified aminoalkyl coated CPG (controlled pore glass). CPG beads are commonly used in automated solid phase DNA synthesis. Many surface modifications have been attached to CPG for introduction of functional groups on the 3′-terminus of synthetic oligonucleotides. For example, reaction conditions developed for CPG (succinic anhydride, pyridine) was used to prepare succinylated AP-GFF (succ-AP-GFF). After copious washing and drying, succ-AP-GFF was tested for residual amino groups using the DMT assay. As described earlier, amine density was almost below the limit of detection of the assay (2 nmole/cm²). AP-GFF was also succinylated using succinic anhydride in DMF and triethylamine to drive the reaction. In either case, excess solvents or reagents were removed as usual and vacuum dried. These methods for preparing succ-AP-GFF are described herein. The discs are stable and easily stored after vacuum drying.

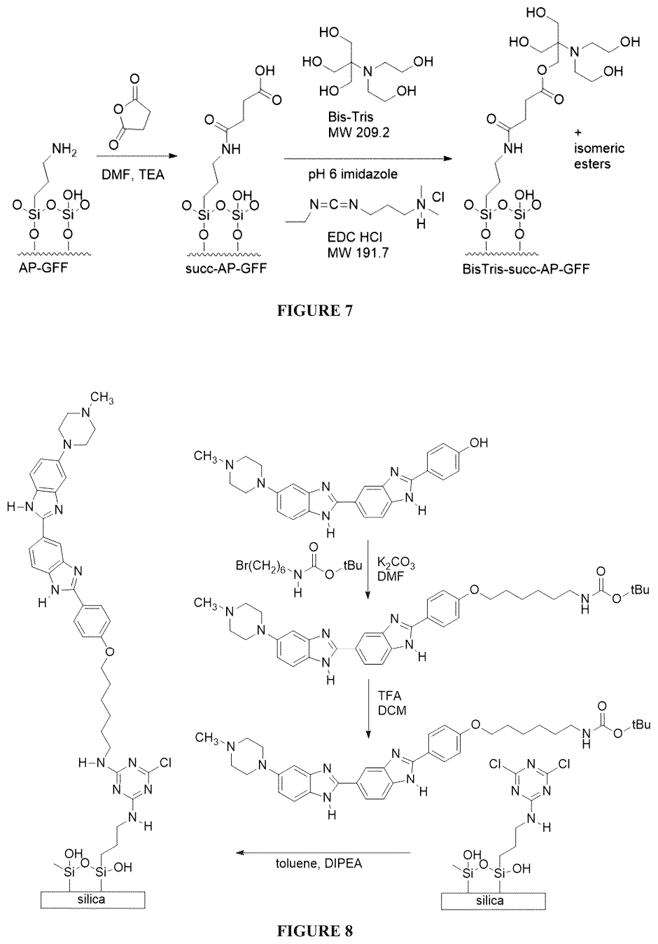

Conjugation of DNA binding ligands to succinylated GFF. Peptide bond formation on glass supports is best executed using anhydrous organic solvents and is generally more efficient than aqueous methods since competing hydrolysis reactions are eliminated. However, some ligands are not soluble in organic solvents and aqueous solutions are required. The water soluble BisTris DNA binding ligand was successfully conjugated to succ-AP-GFF using EDC at pH 6. EDC conjugation at pH 6 promotes reaction of hydroxyl groups of Bis-Tris to form ester bonds as shown in FIG. 7. The resulting BisTris-GFF was shown to capture dsDNA (weakly) at pH 3, but eluted efficiently with pH 8.6 TET. Capture (passthrough) showed Ct 34.1. But elution also showed DNA (Ct 33.8). Both were similar to control (Ct 32.2). Based on the results it appears 25% DNA got captured (2 Ct delay), and then that 25% was fully eluted (2 Ct delay). The amine group in BisTris has pKa=6.46, which allows DNA elution at lower pH.

Minor groove binders (MGB) on GFF. Bis-benzimide (BB) molecules are well known fluorogenic DNA binding molecules. Also known as Hoechst dyes, they are used to stain DNA in cells for fluorescent microscopy. BB dyes have been shown to bind tightly in the minor groove of dsDNA. To explore this novel method of capturing DNA, a hexylamine modified BB dye (BB—NH₂) was prepared and an efficient method to immobilize it to AP-GFF was developed (FIG. 8).

Epoxide coated GFF. Two methods were used to immobilize amine containing ligands. First, the previously described epoxide coating was applied to GFF (EP-GFF). The presence of reactive epoxides on the surface was demonstrated by further treatment with propylene diamine (PDA). The conjugated propylamine groups were measured using the DMT assay and gave amine density of only 15 nmole/cm² after 22 h reaction with PDA (Table 4). The epoxide groups ring open with PDA during reaction with primary amine ligands to generate a secondary amine and secondary alcohol in the linker structure. Despite the low surface density, EP-GFF was treated overnight with BB—NH₂ and tested for amine density and DNA extraction performance. This secondary amine has pKa ˜10 and is acylated by the PFP ester in the DMT assay (12 nmole/cm²). The DNA extraction performance of BB-EP-GFF was poor, perhaps due to the low density.

Cyanuric chloride coated GFF. Cyanuric chloride is a simple linker that can be used to couple two alkylamine containing molecules. The first alkylamine reacts rapidly to displace a single Cl on the trichlorotriazine ring. The resulting dichlorotriazine (see FIG. 8) is de-activated, but reacts slowly with a second alkylamine to displace a second Cl. The resulting monochlorotriazine is unreactive. Cyanuric coated GFF (CC-AP-GFF) was prepared by treating AP-GFF with 0.1 M CC in toluene for 3 h. Similar to silanization Method B, the toluene solution of CC was rinsed off and CC-AP-GFF was washed with toluene, methanol, DCM and dried under vacuum. The CC-AP-GFF was further characterized by testing amine reactivity with propylene diamine as described for epoxide-GFF. Surprisingly, the cyanuric chloride activated surface gave good amine density after reaction with PDA (Table 4, 27 nmole/cm²). CC-AP-GFF was next treated overnight with 1 mL of 0.1 M BB—NH₂ in DMF and 1 mL of Solution B (Method B). The CC was allowed to react overnight and BB-CC-AP-GFF was washed and dried as usual. A 1% inch diameter disc was tested for amine content. As expected, the amine density of the BB coated GFF was close to background level (8 nmole/cm²), indicating no reaction of the PFP ester with immobilized BB.

DNA extraction performance of the BB-CC-AP-GFF discs was tested in the PCR assay. Extraction Score was X, indicating good DNA binding but no DNA release. The rigid triazine linker structure may provide increased access of the BB ligand to dsDNA, therefore improved DNA binding performance vs. the aliphatic EP-GFF linker. It is surprising that the 50 mM KOH was not sufficient to release the captured dsDNA from the minor groove binding BB surface. Perhaps unreacted CC-groups remain on the surface and covalently capture DNA (compare DNA extraction of freshly prepared CC-AP-GFF, Solution B treated CC-AP-GFF and BB-CC-AP-GFF). It is also possible that the BB surface density and DNA binding affinity is too high (try more dilute BB—NH₂). Capping unreacted chloro groups on CC-AP-GFF with aminoethanol may also help improve release of captured DNA from the CC-AP surface. In summary, CC coated GFF provides a novel method for immobilizing amine containing ligands to aminoalkylated glass supports.

TABLE 4
Amine density of coated GFF

| | Amine density | |
|---|---|---|
| GFF surface/name | (nmole/cm²) | DNA extraction score |
| Aminopropyl/AP-GFF | 59 | 12 (high binding, high |
| | | release) |
| Succinate/Succ-AP-GFF | 2 | 0.4 (low binding, low release) |
| Diamine/PDA-EP-GFF | 15 | ND |
| Monoamine/PDA-CC- | 27 | ND |
| AP-GFF | | |
| BisBenzimide/BB-EP- | 12 | −4 (low binding, low release) |
| GFF | | |
| BisBenzimide/BB-CC- | 8 | X (high binding, very low |
| AP-GFF | | release) |
| CL53-GFF (Method B) | 32 | 12* |
| BB-CC-CL53-GFF | ND | 10* |
| GFF | 9 | ND |

All discs were Whatman. EP: Epoxide silanized using Method B. CC: cyanuric chloride activated AP-GFF. PDA: propylene diamine reaction. Amine density measured using DMT assay. Elution for PCR assay used 50 mM KOH. DNA Extraction Score = (Ct of Passthrough − Ct of Elution).
*Elution for PCR assay used pH 8.6 TET buffer, heating for 10 min at 95° C.

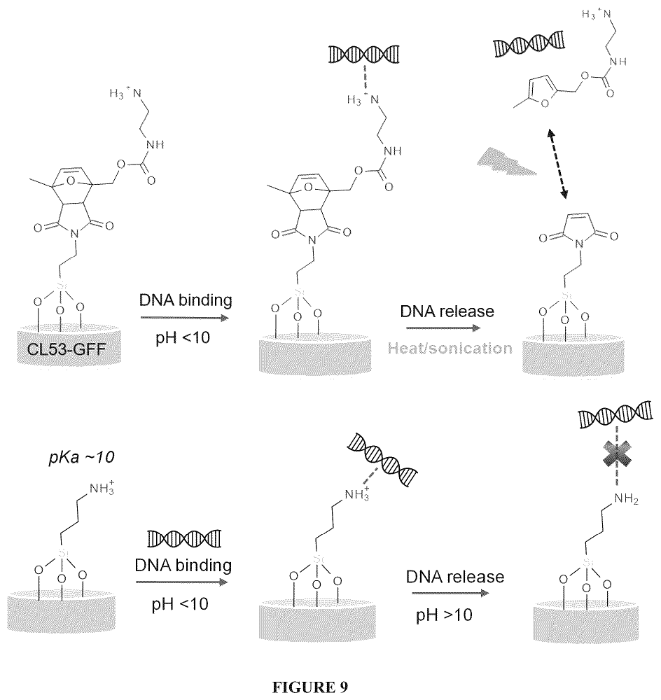

Heat Cleavable Linker for release of Bis-benzimide captured dsDNA. The heat cleavable linkers described above (Table 3) allow release of strongly bound DNA. The linker is cleaved via a reverse Diels-Alder reaction as shown in FIG. 9.

GFF was silanized with a heat cleavable alkylamine linker (CL53) to give CL53-GFF (Whatman filters, Method B). The amine density was measured as usual (32 nmole/cm²), and DNA capture performance was measured with the PCR assay (extraction score=12). CL53-GFF was further activated with cyanuric chloride and conjugation to BB—NH₂ as described for AP-GFF (FIG. 7) to give BB-CC-CL53-GFF. The minor groove binding Bis-Benzimide ligand captures double stranded DNA efficiently as was demonstrated for the non-cleavable BB-CC-AP-GFF. But the cleavable CL53 allowed DNA heat release (extraction score=10) whereas the AP linker did not (extraction score=X). Upon heating, the CL53 linker de-cyclizes to cleave the BB ligand and the glass fiber surface. A maleimide containing fragment remains attached to the glass surface. The cleaved fluorogenic Hoechst dye (and attached furan containing fragment) remains bound to the minor groove of released dsDNA and the complex dissolves into solution. This novel DNA extraction process allows the concentration of eluted BB-labeled DNA to be determined by solution phase fluorescence measurement. Fluorescent properties of the released BB dye are analogous to Hoechst 33342 (ε₃₄₃ (MeOH)=47 mM⁻¹ cm−¹, Ex=361 nm, Em=486 nm), and there are several references for measuring dsDNA concentration of biological solutions with Hoechst 33342.

Stoichiometry of amine density and dsDNA binding capacity. The dsDNA (plasmid) binding capacity of unmodified GFF discs (30 mm diameter) from bacterial lysate using 2 M guanidine hydrochloride has been reported to be 30 μg/cm². The same porosity GFF was coated with DNA binding ligands, but the DNA and RNA binding capacity were not determined with modified GFF. With similar dsDNA binding capacity, an amine density of 50 nmoles/cm² indicates a binding ratio of 1 alkylammonium ion/base pair of DNA. The MGB type ligands should require lower density since a single BB molecule binds a 5 bp DNA segment with high affinity. It is believed that the alkylamines, imidazole, Bis-Tris or BB coated GFF described herein can isolate NA from complex biological samples. These novel GFF will be valuable if cell lysis can be simplified (no GuHCl), processing time can be shortened, or NA quality can be improved (fewer PCR inhibitors, more specific hybridization).

Evaluation of hgDNA from plasma on APTES Modified GFF

The capability of modified glass fiber filter bound to APTES reagent to bind and recover hgDNA from human plasma samples was assessed. The human plasma was treated with a 1/10 dilution of Proteinase K (˜2 mg/ml) and incubated for 15 minutes at room temperature. The samples were then processed with a CTNG assay bead set which contains oligo sets for the Sample Adequacy Control (SAC) of hgDNA. One milliliter of this plasma and proteinase K sample was processed with by the CTNG assay. This assay uses a 4.5M guanidine thiocyanate buffer in combination with polyethylene glycol for DNA purification and uses a glass fiber filter that is not bound to APTES reagent. One milliliter of this sample was also processed with a procedure specifically formulated for the modified filters which does not use polyethylene glycol. The results are provided in the table below.

TABLE 5
GFF Modifications and hgDNA (SAC Signal
from hgDNA in Human Plasma Samples

| Human Plasma | Sample | | | | |
|---|---|---|---|---|---|
| & 1/10 | Volvume | S.P. | SAC | SAC | Pressure |
| Proteinase K | (μL) | Time | Ct | EPF | (psi) |
| RCC CTNG | 1000 | 7′04 | 43.1 | 26 | 50.1 |
| Unmodified GFF without | 1000 | 2′17 | 43.7 | 38 | 23.3 |
| PEG200 | | | | | |
| AP-GFF 2% Modified | 1000 | 2′17 | 37.2 | 134 | 26.3 |
| Filter without PEG200 - | | | | | |
| Reel Method | | | | | |
| APTES 2% Modified | 1000 | 2′17 | 44.2 | 19 | 18.3 |
| Filter without PEG200 - | | | | | |
| Hanging Strip Method | | | | | |

Evaluation of hgDNA from Plasma on APTES Modified GFF

Aminopropyltriethoxylsilane (APTES) was mixed with glass fiber filters to chemically modify the glass surface via silanization. The glass fibers were rinsed with 200 proof ethanol, dried, and then built into a modified GeneXpert® system sample cartridge. The modified cartridges were used in the experiments below.

In a first experiment, a solution of 50 mM Tris, 0.1 mM EDTA and 0.1% Tween 20 was used in the modified filter cartridges in two chambers. In particular, 250 μL of the solution along with 1 μg hgDNA was placed in a chamber A where the mixture was then then passed through the modified filter. In another chamber B, the solution (without hgDNA) was mixed with 20 μL dilute picogreen dye and the filtered solution of hgDNA before entering the PCR tube for fluorescent readings. A standard curve was generated based on fluorescent reading when the hgDNA was placed in chamber B instead of chamber A. By making that switch, the standard curve conditions of hgDNA did not pass through the modified filters. Therefore, differences in the hgDNA read were due to the APTES modified filters having captured the hgDNA on the surface of the filters. The results are provided in the table below.

TABLE 6
Passthrough RNA assessed in GeneXpert

| 1 μg hgDNA processed within | % of |
|---|---|
| 250 μL in modified cartridges | 1 μg DNA |
| Unmodified GFF | 77% |
| >50% GF AP-GFF (Reel Method) | 2% |
| >50% GF APTES (Hanging Strip Method) | 1% |

In a second experiment, a solution of 50 mM Tris, 0.1 mM EDTA and 0.1% Tween 20 was used in the modified filter cartridges in two chambers. In particular, 250 μL of the solution along with 10 μg hgDNA was placed in a chamber A where the mixture was then then passed through the modified filter. In another chamber B, the solution (without hgDNA) was mixed with 20 μL dilute picogreen dye and the filtered solution of hgDNA before entering the PCR tube for fluorescent readings. A standard curve was generated based on fluorescent reading when the hgDNA was placed in chamber B instead of chamber A. By making that switch, the standard curve conditions of hgDNA did not pass through the modified filters. Therefore, differences in the hgDNA read were due to the APTES modified filters having captured the hgDNA on the surface of the filters. The results are provided in the table below.

TABLE 7
Passthrough RNA assessed in GeneXpert

| 10 μg DNA Processed within | % of |
|---|---|
| 250 μL in Modified Cartridges | 10 μg DNA |
| Unmodified GFF | 63% |
| >50% GF AP-GFF (Reel Method) | 2% |
| >50% GF APTES (Hanging Strip Method) | 2% |

In a third experiment, a solution of 50 mM Tris, 0.1 mM EDTA and 0.1% Tween 20 was used in the modified filter cartridges in two chambers. In particular, 250 μL of the solution along with 1 μg rRNA was placed in a chamber A where the mixture was then then passed through the modified filter. In another chamber B, the solution (without rRNA) was mixed with 20 μL dilute ribogreen dye and the filtered solution of rRNA before entering the PCR tube for fluorescent readings. A standard curve was generated based on fluorescent reading when the rRNA was placed in chamber B instead of chamber A. By making that switch, the standard curve conditions of rRNA did not pass through the modified filters. Therefore, differences in the rRNA read were due to the APTES modified filters having captured the rRNA on the surface of the filters. The results are provided in the table below.

TABLE 8
Passthrough RNA assessed in GeneXpert

| | |
|---|---|
| 1 μg rRNA Processed within | % of |
| 250 μL in Modified Cartridges | 1 μg rRNA |

Passthrough Condition (rRNA passed through cartridge in
50 mM Tris + 0.1 mM EDTA + 50 U/mL RNase Inh)

| | |
|---|---|
| Unmodified GFF | 86% |
| >50% GF AP-GFF (Reel Method) | 4% |
| >50% GF APTES (Hanging Strip Method) | 2% |

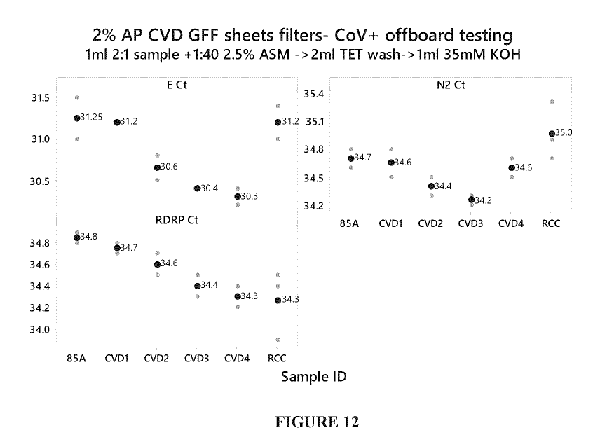

Chemical Vapor Deposition (CVD) method for manufacturing modified glass fibers. GFF sheets were hung in vacuum oven. The entire cleaning, dehydration, and deposition process was performed at 150° C. Briefly, glass fiber surfaces were first plasma cleaned. This surface cleaning was followed by a dehydration purge to remove residual water from the surfaces. An aminosilane (e.g., APTES) was then introduced into the sealed chamber, raising the pressure of the deposition chamber to 2-3 Torr. The reaction time of the surface with the gas phase adsorbate was 5 min. After the deposition, three purge cycles were performed, which consisted of addition of N₂ gas, followed by evacuation. These purge cycles were performed for both safety reasons and also to improve the quality of the deposition-they are used to remove residual silane from the chamber before it is opened, and they aid in removing any unreacted silane from the surfaces of the substrates. FIG. 12 shows CVD modified sheets (CVD 1-4) having better or equal performance to ethanol soak method (85A) or control.

Functional testing of Amine Modified cartridges with Covid Plus and CTNG Assays. The capability of modified glass fiber filter bound to APTES reagent to process and assess Covid-19 targets in contrived clinical nasopharyngeal swab matrix assay and CTNG targets in Zeptometrix matrix was assessed. Cartridges with modified glass fiber filter were built: Cartridge 116A has 2% AP-GFF precut strips; Cartridge 116B had 2% AP-GFF precut strips; Cartridge 116C had 2% AP-GFF precut sheets+separation with glass stir rod; and Cartridge 116D had 2% AP-GFF precut sheets. 300 uL contrived sample was processed in these Covid-19 and CTNG assays with 4.5M GTC lysis buffer, 50 mM tris, 0.1 mM EDTA, 0.1% Tween 20 wash buffer reagent, and a pH 12 40 mM KOH elution reagent. The elution buffer was neutralized with a 400 mM Tris HCl bead. The results are provided in the tables below.

TABLE 9
GFF Modifications and Covid Plus Sample in Clinical Nasopharyngeal Swab Matrix

| | | | | | | | | | | | |
|---|---|---|---|---|---|---|---|---|---|---|---|
| Zeptometrix NATROL in | Sample | S.P. | | | N2 | N2 | RdRP | RdRP | SPC | SPC | |
| Clinical Matrix | Vol | Time | E Ct | E EPF | Ct | EPF | Ct | EPF | Ct | EPF | Pressure |

Unmodified Covid+ Sample Prep - Control

| | | | | | | | | | | | |
|---|---|---|---|---|---|---|---|---|---|---|---|
| 125 uL Sample Processed | 125 | 4′08 | 32 | 680 | 36.1 | 126 | 35.1 | 377 | 32.1 | 133 | 50 |

Unmodified Cart A+ w/PEG200

| | | | | | | | | | | | |
|---|---|---|---|---|---|---|---|---|---|---|---|
| 125 uL with Unmodified Filter | 125 | 2′10 | 33 | 786 | 36 | 1.54 | 35 | 515 | 28.9 | 249 | 35 |

AP-GFF 2% Modified Filter w/o PEG200

| | | | | | | | | | | | |
|---|---|---|---|---|---|---|---|---|---|---|---|
| 300 uL with Modified Filter | 300 | 1′30 | 32 | 1067 | 36.3 | 127 | 34.9 | 543 | 30 | 316 | 24 |

EDA 2% Modified Filter w/o PEG200

| | | | | | | | | | | | |
|---|---|---|---|---|---|---|---|---|---|---|---|
| 300 uL with Modified Filter | 300 | 1′30 | 32 | 993 | 35.1 | 142 | 34.2 | 456 | 29.1 | 279 | 20 |

DETA 2% Modified Filter w/o PEG200

| | | | | | | | | | | | |
|---|---|---|---|---|---|---|---|---|---|---|---|
| 300 uL with Modified Filter | 300 | 1′30 | 34 | 761 | 38.8 | 75 | 36.8 | 332 | 32.8 | 260 | 21 |

TABLE 10
GFF Modifications and CTNG Sample in Clinical Matrix Time

| | | | | | | | | | | | |
|---|---|---|---|---|---|---|---|---|---|---|---|
| CTNG Assay Cells in | Sample | S.P. | CT1 | CT1 | NG2 | NG2 | NG4 | NG4 | SPC | SPC | |
| GuHCl + ASM | Vol | Time | Ct | EPF | Ct | EPF | Ct | SPF | Ct | EPF | Pressure |

RCC Xpress CTNG w/PEG200 - Control

| | | | | | | | | | | | |
|---|---|---|---|---|---|---|---|---|---|---|---|
| RCC Cartridge (no glass beads) | 1000 | 7′04 | 34.9 | 130 | 33.2 | 410 | 33.4 | 202 | 31.6 | 168 | 54 |

Unmodified Cart A+ w/PEG200

| | | | | | | | | | | | |
|---|---|---|---|---|---|---|---|---|---|---|---|
| Unmodified A+ <50% Glass Fill | 1000 | 4′00 | 35.8 | 174 | 33.6 | 576 | 32.7 | 287 | 31.4 | 212 | 75 |

AP-GFF 2% Modified Filter w/o PEG200

| | | | | | | | | | | | |
|---|---|---|---|---|---|---|---|---|---|---|---|
| Modified <50% Glass Fill | 1000 | 2′17 | 34.9 | 281 | 34.6 | 701 | 34.1 | 333 | 32.1 | 301 | 25 |

EDA 2% Modified Filter w/o PEG200

| | | | | | | | | | | | |
|---|---|---|---|---|---|---|---|---|---|---|---|
| Modified <50% Glass Fill | 1000 | 2′17 | 35.0 | 324 | 35.0 | 838 | 33.7 | 394 | 31.1 | 371 | 23 |

DETA 2% Modified Filter w/o PEG200

| | | | | | | | | | | | |
|---|---|---|---|---|---|---|---|---|---|---|---|
| Modified <50% Glass Fill | 1000 | 2′17 | 36.5 | 294 | 36.6 | 701 | 35.9 | 319 | 33.9 | 338 | 25 |

Unmodified Xpress CTNG w/PEG200 - Control

| | | | | | | | | | | | |
|---|---|---|---|---|---|---|---|---|---|---|---|
| Unmodified | 1000 | 7′04 | 35 | 130 | 33.2 | 410 | 33.4 | 202 | 31.6 | 168 | 54 |

Unmodified Cart A+ w/PEG200

| | | | | | | | | | | | |
|---|---|---|---|---|---|---|---|---|---|---|---|
| Unmodified <50% Glass Fill | 1000 | 4′00 | 36 | 174 | 33.6 | 576 | 32.7 | 287 | 31.4 | 212 | 75 |

AP-GFF 2% Modified Filter w/o PEG200

| | | | | | | | | | | | |
|---|---|---|---|---|---|---|---|---|---|---|---|
| Modified <50% Glass Fill | 1000 | 2′17 | 35 | 281 | 34.6 | 701 | 34.1 | 333 | 32.1 | 301 | 25 |

EDA 2% Modified Filter w/o PEG200

| | | | | | | | | | | | |
|---|---|---|---|---|---|---|---|---|---|---|---|
| Modified <50% Glass Fill | 1000 | 2′17 | 35 | 324 | 35 | 838 | 33.7 | 394 | 31.1 | 371 | 23 |

DETA 2% Modified Filter w/o PEG200

| | | | | | | | | | | | |
|---|---|---|---|---|---|---|---|---|---|---|---|
| Modified <50% Glass Fill | 1000 | 2′17 | 37 | 294 | 36.6 | 701 | 35.9 | 319 | 33.9 | 336 | 25 |

Functional Testing of modified glass fiber filters with removal of chaotropic lysis buffer. The ability of modified glass fiber filters to purify DNA and RNA is advantageous with respect to the lysis solution used. The lysis solution is not required to contain high salt content and it is not required to contain polyethylene glycol or alcohol. The data below shows the successful purification of viral RNA with modified glass fiber filters without the addition of chaotropic salt or polyethylene glycol. Particularly, when the viral particles are within a solution containing 2% Brij58 detergent, the viral particles are readily lysed by detergent solubilization and the RNA is free to bind to the modified glass fibers.

TABLE 11
GFF Modifications and Covid Plus Sample in the absence or presence of chaotropic reagent

| | | | | | | | | |
|---|---|---|---|---|---|---|---|---|
| Zeptometrix NATROL in | | | | | | | | |
| Artificial Sample Matrix | Sample Vol | S.P. Time | E Ct | E EPF | N2 Ct | N2 EPF | RdRP Ct | RdRP EPF |

Unmodified Covid+ Sample Prep - Control

| | | | | | | | | |
|---|---|---|---|---|---|---|---|---|
| 4.5M GTC Lysis | 125 | 4′08 | 32 | 618 | 34.8 | 114 | 34.1 | 359 |

AP-GFF 2% Modified Filter w/o PEG200

| | | | | | | | | |
|---|---|---|---|---|---|---|---|---|
| 0M GTC, 2% Brij58, | 300 | 2′10 | 31 | 1126 | 35.4 | 176 | 34.9 | 455 |
| 10 mM EDTA | | | | | | | | |

The use of the modified glass fibers allowed for the processing of higher volumes as well as the reduction in chaotropic agent and binding agent, as shown in tables 9-11 above. Overall, these advantages provide a sample prep time (S.P. Time) advantage due to the use of fewer reagents for DNA/RNA purification.

The results shown above indicate that the amine modified glass fiber filter cartridges perform comparably or better than other cartridge types (such as RCC—revised cartridge C, see for example WO2021263101A1 and WO2015013676A1 which utilize RCC cartridge to process SARS-CoV2 and CTNG samples, respectively). The simplified reagent chemistry afforded by the amine modified surfaces allow for faster time to result, elimination of several types of chemicals in the sample prep, and a higher sensitivity RT-PCR assay due to reduced reagent carryover.

Evaluation of hgDNA and CT/NG from Human Urine on APTES Modified GFF.

The capability of modified glass fiber filter bound to APTES reagent to bind and recover hgDNA and CT/NG from human urine samples was assessed. The samples were processed with CTNG assay bead set which contains oligo sets for the Sample Adequacy Control (SAC) of hgDNA, Chlamydia trachomatis and Neisseria gonorrhoeae. One milliliter of this sample was processed using a 4.5M guanidine thiocyanate buffer in combination with polyethylene glycol for DNA purification and an unmodified glass fiber filter (not bound to APTES reagent). One milliliter of this sample was also processed with a procedure specifically formulated for the modified filters which does not use polyethylene glycol. The results are provided in the tables 12 and 13 below.

TABLE 12
GFF Modifications and CTNG Sample

| Cartridge | | Sample | | | | | |
|---|---|---|---|---|---|---|---|
| type | Filter Type | Prep Time | CT1 Ct | NG2 Ct | NG4 Ct | SAC Ct | SPC Ct |
| 116B | APTES Modified | 4′00 | 30.7 | 33.8 | 32.6 | 32.1 | 29.8 |
| 116B | APTES Modified | 4′00 | 30.6 | 33.4 | 33.4 | 31.8 | 29.5 |
| 116B | APTES Modified | 4′00 | 30.5 | 34 | 33.3 | 31.4 | 29.8 |
| 116B | APTES Modified | 4′00 | 31.2 | 34.3 | 34.1 | 32.3 | 30 |
| RCC | Unmodified | 9′30 | 31.7 | 33.2 | 33.6 | 33.4 | 31.4 |
| RCC | Unmodified | 9′30 | 31.4 | 33.2 | 32.6 | 33.1 | 31.2 |
| RCC | Unmodified | 9′30 | 32.5 | 34.1 | 33.8 | 33.7 | 32.1 |
| RCC | Unmodified | 9′30 | 31.4 | 33.4 | 32.7 | 32.9 | 30.9 |

TABLE 13
GFF Modifications and CTNG Sample

| Cartridge | | Sample | | | | | |
|---|---|---|---|---|---|---|---|
| type | Filter Type | Prep Time | CT1 EPF | NG2 EPF | NG4 EPF | SAC EPF | SPC EPF |
| 116B | APTES Modified | 4′00 | 288 | 716 | 402 | 266 | 269 |
| 116B | APTES Modified | 4′00 | 313 | 929 | 351 | 338 | 332 |
| 116B | APTES Modified | 4′00 | 287 | 798 | 303 | 310 | 311 |
| 116B | APTES Modified | 4′00 | 334 | 846 | 358 | 333 | 346 |
| RCC | Unmodified | 9′30 | 161 | 513 | 195 | 143 | 179 |
| RCC | Unmodified | 9′30 | 221 | 465 | 243 | 202 | 202 |
| RCC | Unmodified | 9′30 | 145 | 315 | 171 | 127 | 136 |
| RCC | Unmodified | 9′30 | 235 | 507 | 267 | 191 | 234 |

Capture Efficiency of Modified Glass Fiber Filters with Large Volume Plasma Samples.

Further experiments were carried out to extract cell free DNA from EDTA plasma. For the Revised Cartridge C protocol, 1 mL plasma was mixed with 100 ul Proteinase K (˜20 mg/ml, Roche). The mixture was vortexed for 10-15 secs and incubated at RT for 1 min. 2 ml Lysis Reagent was added and further vortexed for 10-15 secs and incubated at RT for 10 mins. The resulting 4.5 ml lysate was loaded onto the GX instrument and the remaining steps of DNA purification were carried out using standard reagents from Cepheid commercial assays. For the modified glass fiber filter cartridges, 2 ml of plasma were mixed with 100 ul Proteinase K (˜20 mg/ml, Roche). The mixture was vortexed for 10-15 secs and incubate at RT for 10 min. 2 ml Lysis Reagent (5M GuHCl, 100 Tween-20 and 0.0075% SE-15 defoamer) was added and further vortexed for 10-15 secs and incubated at RT for 10 mins. The resulting ˜4 ml lysate was loaded onto the GX instrument and the remaining steps of DNA purification were carried out using standard reagents from Cepheid commercial assays. Extracted DNA from 1 ml of plasma (RCC) or 2 ml of plasma (ModGFF) were measured using a digital PCR ESR1_E380Q mutation detection assay. As indicated below the amounts of WT and mutant alleles representing cell free DNA were detected were at levels about 2× higher when processing 2 ml of plasma on ModGFF vs. 1 ml of plasma processed using Revised Cartridge C. One exception was the ModGFF-1 cartridge due to variability in the processing efficiency.

TABLE 14
GFF Modifications and Cell Free DNA Samples

| | | ddPCR | | Eluate | Copy |
|---|---|---|---|---|---|
| Sample | | reads AVG | Dilution | volume | number per |
| Carts | Targets | (cpn/ul) | factor | (ul) | per cart |
| ModGFF-1 | E380Q | 53.4 | 2.2 | 65.0 | 7710 |
| ModGFF-2 | E380Q | 83.5 | 2.2 | 65.0 | 12065 |
| ModGFF-4 | E380Q | 80.8 | 2.2 | 65.0 | 11669 |
| ModGFF-5 | E380Q | 72.4 | 2.2 | 65.0 | 10454 |
| ESR1 RCC-1 | E380Q | 22.0 | 2.2 | 70.0 | 3429 |
| ESR1 RCC-2 | E380Q | 28.5 | 2.2 | 70.0 | 4436 |
| ESR1 RCC-3 | E380Q | 15.9 | 2.2 | 70.0 | 2478 |
| ModGFF-1 | WT | 26.7 | 2.2 | 65.0 | 3856 |
| ModGFF-2 | WT | 40.6 | 2.2 | 65.0 | 5869 |
| ModGFF-4 | WT | 39.7 | 2.2 | 65.0 | 5741 |
| ModGFF-5 | WT | 35.3 | 2.2 | 65.0 | 5103 |
| ESR1 RCC-1 | WT | 14.8 | 2.2 | 70.0 | 2305 |
| ESR1 RCC-1 | WT | 17.9 | 2.2 | 70.0 | 2778 |
| ESR1 RCC-1 | WT | 12.4 | 2.2 | 70.0 | 1929 |

Modified GFF Capture Efficiency of Target from Over 3 mL Whole Blood.

Genomic Strep DNA was spiked into fresh blood to a final concentration of 500 copies/mL. Blood was then mixed in a ratio of 1 part blood: 0.5 parts enzymatic agent (proteinase K): 0.91 parts chaotropic agent (5M GuHCl) and allowed to sit at room temperature for 20 minutes to form sample lysate. Sample lysate was then run through a 25 mm diameter 10 micron polypropylene disc using a 12 mL syringe. Sample lysate was then loaded into two chambers (having a total volume of 6.32 mL) of a cartridge followed by filtering through a 2%-APTES GFF within the cartridge. By processing over 6 mL (6.32 mL) of sample lysate, over 3 mL of blood (3.225 mL) was processed by a single filter. The waste sample was then collected and loaded into two chambers of a second cartridge, where it was then again run through a modified GFF. The filter of both cartridges was rinsed with TET and eluted using high pH KOH followed by conducting PCR. See Table 15 for the Ct results for both cartridges.

TABLE 15
Capture efficiency of modified glass fiber
filters with large volume blood samples

| | Strep A Ct | Strep A EPF |
|---|---|---|
| Cartridge sample | 28.425 | 573.25 |
| Passthrough sample | 33.9 | 371.25 |
| Delta: | +5.475 | −202 |

The resulting Ct from each cartridge can be compared. The Ct from the first cartridge corresponds to how efficiently the target DNA was captured from blood and then released into PCR by a modified GFF. The Ct from the second cartridge provides details as to how many copies of target DNA were able to bypass capture on the initial filter, and can be considered as the “passthrough” result. Using approximations between Strep A copy number and observed Ct based on LOD studies from the commercial Strep A assay, a delay of >5 Ct between the initial modified GFF and the second modified GFF indicates that only ˜2% as many Strep DNA copies were present in the passthrough PCR compared to the PCR run from the eluate of the first filter (or, that 40× as much Strep DNA was efficiently captured on the initial modified GFF compared to the second). This indicates a high efficiency of the modified GFF at extracting pathogen DNA from large volumes of human genomic DNA.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

1. A separating material for nucleic acid isolation comprising:
a glass fiber solid support comprised of borosilicate glass and a compound bonded to the glass fiber solid support, the compound being derived from a structure represented by the formula:
Y-(L)_{y}-SiX₃
wherein,
Y is a DNA binding ligand selected from an alkylamine, a cycloalkylamine, an alkyloxy amine, a polyamine moiety, an arylamine, an intercalating agent, a DNA groove binder, a peptide, an amino acid, a protein, or a combination thereof,
L is a linker selected from an alkyl group, a heteroalkyl group, an alkene group, a heteroalkene group, a polyacrylic acid, a Diels-Alder adduct, or a combination thereof,
each X, independently for each occurrence, is selected from a hydrolyzable group, an alkyl group, a heteroalkyl group, an alkenyl group, or two or three Xs combine to form one or more cyclic groups, or one X combines with Y to form a cyclic azasilane, and
y is 0 or 1.

2. A separating material for nucleic acid isolation comprising:
a glass fiber solid support comprising a Diels-Alder adduct having a DNA binding ligand, cyanuric chloride, or a combination thereof,
wherein the adduct or cyanuric chloride is chemically bonded to the glass fiber solid support, optionally via a linker.

3. The separating material of claim 2, wherein the DNA binding ligand comprises an amine group, an intercalating agent, a minor groove binder, a peptide, an amino acid, a protein, or a combination thereof, preferably an alkylamine, a cycloalkylamine, an alkyloxy amine, a polyamine moiety, an arylamine, an intercalating agent, a DNA groove binder, a peptide, an amino acid, a protein, or a combination thereof.

4. The separating material of claim 1, wherein the DNA binding ligand comprises an alkylamine group, an imidazole group, a bisbenzimide minor groove binder, or a combination thereof.

5. The separating material claim 1, wherein the DNA binding ligand is selected from spermine, methylamine, ethylamine, propylamine, ethylenediamine, diethylene triamine, 1,3-dimethyldipropylenediamine, 3-(2-aminoethyl)aminopropyl, (2-aminoethyl)trimethylammonium hydrochloride, tris(2-aminoethyl)amine, or a combination thereof.

6. The separating material claim 1, wherein the Diels-Alder adduct is derived from an unsaturated cyclic imido group.

7. The separating material of claim 1 wherein the Diels-Alder adduct is derived from a structure represented by the general Formula, 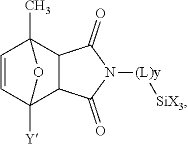
their isomers, salts, tautomers, or combinations thereof, wherein
Y′ is the DNA binding ligand,
L is a linker selected from an alkyl group, a heteroalkyl group, an alkene group, a heteroalkene group, a polyacrylic acid, a Diels-Alder adduct, or a combination thereof,
each X, independently for each occurrence, is selected from a hydrolyzable group, an alkyl group, a heteroalkyl group, an alkenyl group, or two or three Xs combine to form one or more cyclic groups, or one X combines with Y to form a cyclic azasilane, and
y is 0 or 1.

8. The separating material of claim 1, wherein the linker, when present, is selected from an alkyleneoxy group, an alkylene group, cyanuric chloride, an alkylamine, or a combination thereof.

9. The separating material of claim 1, wherein at least two Xs are independently selected from a halogen, an alkoxy, a dialkylamino, a trifluoromethanesulfonate, or combine together with the Si atom to which they are attached to form a silatrane, a cyclic siloxane, a polysilsesquioxane, or a silazane, preferably wherein at least two Xs are independently selected from an alkoxy group (such as ethoxy or methoxy).

10. The separating material of claim 1, wherein the compound is derived from one of the following structures: 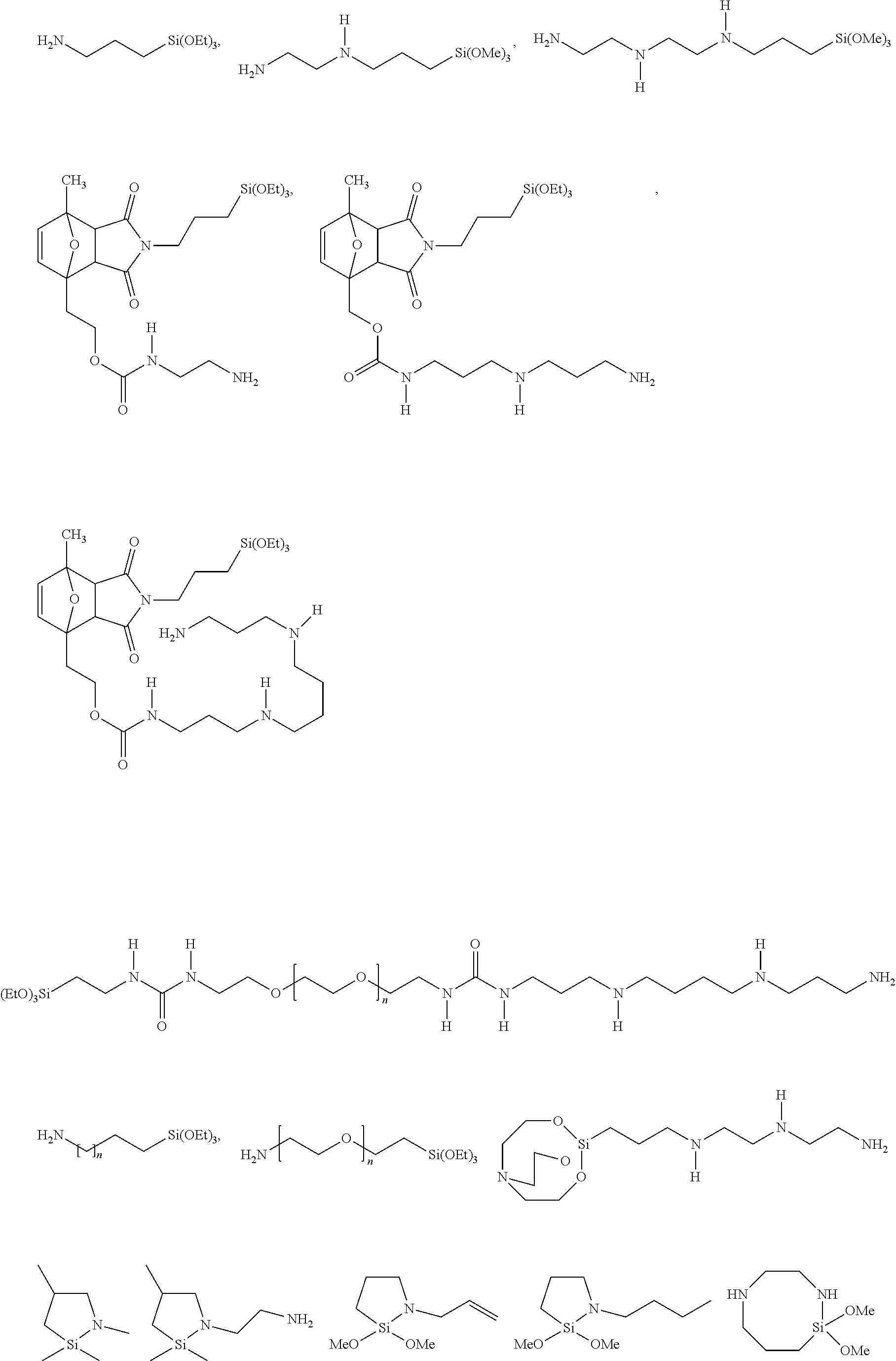
3-aminopropyltrimethoxysilane, an aminoalkylsilatrane, 3-(2-aminoethyl)aminopropyltriethoxysilane, 3-(2-aminoethyl)aminopropyltrimethoxysilane, or a combination thereof, and wherein n is an integer from 0 to 10, from 1 to 10, or from 1 to 5.

11. The separating material of claim 1, wherein
the compound is derived from 3-aminopropyltriethoxysilane, 3-aminopropyltrimethoxysilane, or a combination thereof.

12. The separating material of claim 1, wherein the glass fiber solid support has a surface density of the compound or Diels Alder adduct of 10 nmoles/cm² or greater, 20 nmoles/cm² or greater, 35 nmoles/cm² or greater, or from 30-100 nmoles/cm².

13. The separating material of claim 1, wherein the glass fiber solid support has a DNA binding capacity of at least 10 μg/cm², 20 μg/cm² or greater, 35 μg/cm² or greater, or from 30-100 μg/cm².

14. The separating material of claim 1, wherein the glass fiber solid support has a pore size from 0.2 μm to 3 μm, from 0.2 μm to 2 μm, from 0.5 μm to 1.0 μm, or from 0.6 μm to 0.8 μm.

15. The separating material of claim 1, wherein the glass fiber solid support comprises beads to facilitate mechanical lysis, wherein the beads are selected from glass beads, silica beads, or a combination thereof.

16. The separating material of claim 1, wherein the glass fiber solid support has a basis weight from 35 g/m² to 100 g/m², preferably from 50 g/m² to 85 g/m², or from 70 g/m² to 85 g/m².

17. The separating material of claim 1, wherein the glass fiber solid support has a fiber diameter from 1 μm to 100 μm, preferably from 1 μm to 50 μm, or from 1 μm to 25 μm.

18. The separating material of claim 1, wherein the glass fiber solid support has a thickness from 250 μm to 2,000 μm, from 300 μm to 1,500 μm, from 300 μm to 1,000 μm, from 300 μm to 750 μm, or from 350 μm to 500 μm.

19. A method for isolating a nucleic acid from a biological sample, the method comprising:
(a) causing the nucleic acid to contact a separating material according to claim 1; and
(b) eluting the nucleic acid from the separating material.

20.-37. (canceled)

38. A sample cartridge for isolation and detection of nucleic acid from a biological sample, comprising:
a cartridge body having a plurality of chambers defined therein, wherein the plurality of chambers are in in fluidic communication through a fluidic path of the cartridge, and wherein at least one chamber is configured to receive the biological sample,
a reaction vessel configured for amplification of the nucleic acid by thermal cycling, and
a filter disposed in the fluidic path between the plurality of chambers and the reaction vessel, wherein the filter comprises a separating material according to claim 1,
wherein the plurality of chambers and the reaction vessel independently comprise reagents for releasing nucleic acid from the biological sample, and primers and probes for detection of the nucleic acid.

39. A sample cartridge for isolation and detection of nucleic acid from a biological sample, comprising, comprising:
a cartridge body having a plurality of chambers therein, wherein the plurality of chambers include:
a sample chamber having at least a fluid outlet in fluid communication with another chamber of the plurality;
a lysis chamber in fluidic communication with the sample chamber, the lysis chamber comprising reagents for releasing nucleic acid, optionally wherein the sample chamber and lysis chamber are the same;
a reaction vessel fluidically coupled to the plurality of chambers of the cartridge body and configured for amplification of nucleic acid and ii) detection of a plurality of amplification products;
a filter disposed in the fluidic path between the lysis chamber and the reaction vessel, wherein the filter comprises a solid support modified with a DNA binding ligand selected from an alkylamine, a cycloalkylamine, an alkyloxy amine, a polyamine moiety, an arylamine, an intercalating agent, a DNA groove binder, a peptide, an amino acid, a protein, or a combination thereof, and
a plurality of primers and/or probes disposed in one or more chambers of the plurality of chambers or reaction vessel for detection of the nucleic acid.

40.-56. (canceled)
